(19) United States
(12) Patent Application Publication
(10) Pub. No.: US 2023/0310650 A1
(43) Pub. Date: Oct. 5, 2023

(54) Bacterial Vehicle for Engineering of Non-Phagocytic Immune Cells

(71) Applicant: DANMARKS TEKNISKE UNIVERSITET, Kongens Lyngby (DK)

(72) Inventors: Scott QUAINOO, Kongens Lyngby (DK); Morten Otto Alexander SOMMER, Kongens Lyngby (DK); Sarah Aparecida SOARES, Kongens Lyngby (DK)

(21) Appl. No.: 18/014,649

(22) PCT Filed: Jul. 7, 2021
(86) PCT No.: PCT/EP2021/068734

§ 371 Date: Jan. 5, 2023

(30) Foreign Application Priority Data
Jul. 7, 2020 (EP) 20184439.6

(51) Int. Cl. A61K 48/00; A61K 38/16; A61K 39/00; A61K 31/7105; A61K 38/46; A61P 37/02

(52) U.S. Cl. CPC A61K 48/0025; A61K 38/164; A61K 38/162; A61K 39/4631; A61K 31/7105; A61K 38/465; A61P 37/02

(57) ABSTRACT

The invention provides an invasive recombinant bacterial cell for use in prevention and/or treatment of an immune-related disorder; said bacterial cell comprising one or more recombinant nucleic acid molecule(s) encoding one or more therapeutic agent(s) for use in prevention and/or treatment of said immune-related disease in a mammal in need thereof.

### FIELD OF THE INVENTION

The invention provides an invasive recombinant bacterium for use in prevention and/or treatment of an immune-related disorder; said bacterial cell comprising one or more recombinant nucleic acid molecule(s) encoding one or more therapeutic agent(s) for use in prevention and/or treatment of said immune-related disease in a mammal in need thereof.

### BACKGROUND

The cells of the mammalian immune system can be categorized as lymphocytes (T-cells, B-cells, and natural killer (NK) cells), granulocytes (Eosinophils, neutrophils, and basophils), and monocytes (macrophages and dendritic cells); which together provide resistance to infection, toxins and cancer. T-cells, B-cells, NK-cells together with basophil cells, are non-phagocytic, and comprise the main cells of the adaptive immune response, which includes the production of cytokines, antibodies, and complement proteins.

A wide range of diseases are attributable to the immune system. For example, when the immune system fails to distinguish self- from nonself-antigens, this results in a wide range of chronic autoimmune diseases (AIDs), whereby a self-reactive immune response, mediated by B-cell auto-antibodies and self-reactive T-cells, destroys the body's own tissues. Additionally, various types of cancer incapacitate the immune system, of itself, specifically blood cancers such as leukaemia, Non-Hodgkin lymphoma, Hodgkin lymphoma, B cell acute lymphocyte leukemia (ALL), refractory B cell lymphoma, or multiple myeloma. While the immune system plays a key role in preventing other cancers in its early stages, this protection is limited, since genetic changes amongst the cancer cells enable them to escape the immune system.

Targeted treatment of immune-related disorders is recognized as being essential, in order to avoid off-target serious adverse events, whether it be to restore immune tolerance in autoimmune diseases or to detect and eliminate pathogens or cancers. Genome editing (e.g. CRISPR-Cas9 system) and adoptive immunotherapy are among the tools that facilitate new strategies for targeted therapy, and potentially provide long term disease control. By way of example, T-cells derived from a patient can be engineered ex vivo with a retroviral vector to express a chromosomally encoded chimeric antigen receptor (CAR) that recognizes unique surface antigens expressed by tumor cells. Once re-introduced into the patient, the CAR T-cells bind with their engineered receptors to antigens on the tumor cells which initiate various signaling cascades that activate the CAR T-cells. The activated CAR T-cells then exert a cytotoxic response to recognized cancer cells and attract other immune cells to the site. As the CAR construct is introduced into the T-cell chromosome, proliferating activated CAR T-cells pass on their CAR constructs to daughter cells and therefore mount a sustained treatment effect from potentially a single treatment dose.

The use of adoptive cellular therapy for cancer treatment can be extended to other immune-related disorders. In the case of AIDs, the CAR-T cells are modified to target specific autoantigens or antibodies expressed on a pathogenic cell surface. More specifically, chimeric autoantibody receptor T (CAAR-T) cells are engineered to express a specific antigen that recognizes and binds to cognate autoantibodies expressed by the self-reactive antibody-producing B cells, leading to their elimination. Alternatively, regulatory T-cells (Treg) can be modified ex-vivo into CAR-Tregs having antigen specificity and used to treat AIDs by a pathogenic mechanism (Chen Y et al., 2019). Among the many diseases of the immune system whose treatment employs autologous gene therapy are various forms of immunodeficiency. For example, mutations in the Adenosine deaminase (ADA) gene results in autosomal recessive severe combined immunodeficiency (SCID), where absent or impaired ADA function causes the accumulation of the toxic metabolites adenosine, 2′deoxyadenosine and deoxyadenosine triphosphate (dATP), leading to severe lymphocytopaenia affecting T- and B-lymphocytes and NK cells. Gene therapy treatments rely on ex vivo gene engineering using retroviral vectors.

A common feature for the numerous current cell therapies of immune-related disorders is the need to engineer the target immune cells ex vivo using viral vectors, which adds both to the risks associated with the treatment (e.g.: toxicity of chemo/radio therapy during cell re-introduction, fear of mutating oncogenic viral vectors, etc.) and the technical complexity (e.g.: production bottlenecks of viral vectors, high loads of re-introduced engineered cells leading to excessive anti-/pro-inflammatory responses, lengthy and cumbersome treatment preparation, high treatment cost, etc.). The disadvantages related to current therapy give rise to a need to provide alternative tools and methods that would facilitate genome engineering and/or cellular regulation of cells of a patient's immune system in vivo or ex vivo.

### SUMMARY OF THE INVENTION

An invasive recombinant bacterial cell for use in prevention and/or treatment of an immune-related disorder; said bacterial cell comprising one or more recombinant nucleic acid molecule(s) encoding one or more therapeutic agent(s) for use in prevention and/or treatment of said immune-related disorder in a mammal; wherein said bacterial cell comprises one or more recombinant invasive gene(s) that facilitates invasion and release of said one or more recombinant nucleic acid molecule(s) or said one or more therapeutic agent(s) in a mammalian non-phagocytic immune cell and thereby functions as a bacteria-mediated delivery vector for in vivo or ex vivo delivery of said one or more recombinant nucleic acid molecule(s) or said one or more therapeutic agent(s) to the mammalian non-phagocytic immune cell, and wherein the immune-related disorder preferably is selected from the group: an autoimmune disorder, cancer, and a lymphoproliferative disorder.

In a further aspect thereof, the invasive recombinant bacterial cell for use in prevention and/or treatment of an immune-related disorder, is a cell comprising one or more recombinant invasive gene(s) for expressing protein(s) for invasion of non-phagocytic immune cells, said protein(s) selected from the group:
an 1.B.54 family invasin in combination with a 1.C.12.1.7 family cytolysin;
viral envelope glycoproteins, preferably comprising an HIV-1 glycoprotein 120 and a HIV-1 glycoprotein 41, in combination with a 1.B.12.8.2 family autotransporter-1; and
1.C.36.3.1 Type III secretion proteins, preferably comprising GPI-anchored ipaB and ipaC proteins.

In a further aspect thereof, said mammalian non-phagocytic immune cell is a T-lymphocyte, B-lymphocyte, Natural Killer cell, or basophil.

In a further aspect thereof, said mammalian non-phagocytic immune cell is a member of the group consisting of a primate, bovine, ovine, porcine, feline, buffalo, canine, goat, equine, donkey, and camel cell.

In a further aspect thereof, said therapeutic agent is a recombinant or native DNA, RNA, or protein agent, or a combination thereof. Said agent may be selected from the group: a Chimeric Antigen Receptor; a small interfering RNA; a protein inhibitor of any one of T cell activation; T cell suppression; T cell proliferation and T cell cell death; a protein inducer of any one of T cell activation; T cell suppression; T cell proliferation and T cell cell death; a cytotoxin; a cytokine; a chemokine, and a CRISPR-Cas system.

In a further aspect thereof, said immune-related disorder is selected from the group: autoimmune disorder(s), cancer(s), and lymphoproliferative disorder(s).

In a second aspect the invention provides a method for prevention and/or treatment of an immune-related disorder in a mammal, the method comprising administering to a mammal diagnosed with said immune-related disorder, an invasive recombinant bacterial cell comprising one or more recombinant nucleic acid molecule(s) encoding one or more therapeutic agent(s); wherein said bacterial cell is capable, or engineered, to deliver said recombinant nucleic acid molecule(s) or said therapeutic agent(s) to a mammalian non-phagocytic immune cell. Delivery to said mammalian non-phagocytic immune cell may take place in-vivo, or ex-vivo followed by the step of re-introducing s the immune cell into the mammalian subject from which it was derived.

### DESCRIPTION OF THE INVENTION

### Definitions and Abbreviations

Disorder/Disease: A disease is a pathophysiological response to internal or external factors; while a disorder is a disruption to regular bodily structure and function. For the purpose of the present application the term “disorder” is to be understood to be an umbrella term that encompasses both a disease and a disorder in a mammalian subject that may be treated by the invasive recombinant bacterial cells of the present invention.

EMOPEC: “Empirical model and Oligos for Protein Expression Changes” used to predict Ribosomal Binding Site (RBS) strength (Bonde M T et al., 2016; http://emopec.biosustain.dtu.dk/).

Immune-related disorders are any diseases or disorders that can be treated, prevented or ameliorated by modulating at least one component of the host immune system; including autoimmune disorder(s); cancer(s); infectious disease(s), lymphoproliferative disorder(s), neurological and neurodegenerative diseases, and genetic disorder(s), and optionally somatic genetic disorder(s); and for which the invasive recombinant bacterial cells of the invention may be used as a bacteria-mediated delivery vector for providing said treatment, prevention or amelioration.

Invasive recombinant bacterial cell is a bacterial cell comprising invasive gene(s) or recombinant invasive gene(s) conferring the cell with the capability of ex-vivo and/or in vivo delivery of one or more recombinant nucleic acid molecules or therapeutic agent(s) comprised in said bacterial cell to a mammalian non-phagocytic immune cell.

Non-phagocytic immune cells: as defined herein are non-phagocytic T-cells, B-cells, natural killer cells, and basophils that are components of the mammalian adaptive immune system.

RT: Room temperature

TNP-KLM: 2,4,6, Trinitrophenyl hapten (TNP) conjugated to Keyhole Limpet Hemocyanin protein (KLM) via amide bonds to lysine.

### FIGURES

FIG. 1: Cartoon showing an engineered bacteria-mediated delivery vector delivering therapeutic genetic material or proteins to a non-phagocytic immune cell; where the bacterial vehicle is engineered to express invasion and lysis genes (as illustrated by the two component system Inv-Hly), which facilitate its uptake into a non-phagocytic immune cell, thereby delivering therapeutic recombinant nucleic acid molecule(s) or protein(s) that induce activation or lysis of the invaded immune cell.

FIG. 2: Plasmid maps

FIG. 3: Incucyte analysis of cell invasion: primary human activated T cells were infected with EcN expressing invasive construct ipaBC and cultured in the presence of antibiotics for 1 h 50 min. Post infection culture was performed inside an Incucyte live cell imager and fluorescent microscope images were taken every 30 minutes. a) Fluorescence microscopy images showing a cell containing pHRodo labelled bacteria at 1 h 50 min, with the arrow showing the invaded cell. b) Total integrated red fluorescence intensity average for each images cell is shown at 1 h 50 min. ipaBC+GFP=EcN+pCOLA−ipaBC−inaK+pZE3119−sfgfp, WT=EcN, NC=Uninfected cells, PC=EcN in citrate buffer. Error bars show SEM. N=5.

FIG. 4: Incucyte analysis of inv-hly BACTERIAL INTRACELLULAR DELIVERY VECTOR invasion of Jurkat cells. Jurkat E6-1 cells were infected with invasive EcN-Tn7::GFP at different MOIs and cultured in the presence of antibiotics for several days. Post infection culture was performed inside an Incucyte live cell imager and fluorescent microscope images were taken hourly. a) Images show representative events of intracellular bacterial replication at indicated MOIs. Numbers inside the images indicate DD:HH. b) Quantification of invaded cells where the percentage of cells containing green bacteria but not showing red autofluorescence is shown on the y-axis over time on the x-axis. Cell only where uninfected control cells. Error bars show SEM. N=3.

FIG. 5: Fluorescence microscopy analysis of cell invasion. Jurkat E6-1 (a&c) or human PBMCs (b) were infected for 1 hour with invasive (pSQ11 & pV3) or wild type (WT) EcN strains harbouring a sfGFP plasmid. a) Total and internalised bacteria were labelled with GFP in green, extracellular bacteria with anti-E. coli LPS antibody in red (Atto550), and Jurkat CD49D integrin with anti-CD49D antibody in blue (BV480). b) Total and internalised bacteria were labelled with GFP in green, extracellular bacteria with anti-E. coli LPS antibody in red (Atto550), and cellular DNA with DAPI in blue. c) Total and internalised bacteria were labelled with GFP in green, extracellular bacteria with anti-E. coli LPS antibody pseudocoloured in red (AF350), and Jurkat actin, after permeabilisation, with Rhodamine phalloidin pseudocoloured in blue. Arrows indicate locations of intracellular bacteria.

FIG. 6: inv-hly mediated DNA transfer. Jurkat E6-1 cells were infected with invasive (pGB2Ωinv-hly) E. coli BM2710 carrying an anti-CD3d shRNA-mCherry reporter plasmid or no reporter. After infection, cells were cultured in the presence of antibiotics for several days and imaged in an Incucyte instrument. a) Fluorescence microscopy images following the mCherry expression of an invaded cell over time. mCherry indicates reporter plasmid expression, Cyto green indicated labelling with the Incucyte Cytolight Rapid Green live cell marker. b) Average number of mCherry+& cytolight rapid green+cells per image over time. Error bars show SEM. N=3.

FIG. 7: Inv-hly mediated protein transfer. Jurkat E6-1 cells were infected with invasive (V3) EcN expressing b-lactamase or non-expressign, non-invasive EcN (WT) at MOI 640 or 1280. After infection, cells were incubated in the presence of antibiotics followed by loading with CCF4-AM. Loaded cells were analysed on a flow cytometer to determine percentages of blue cells, indicative of protein transfer. Green vs blue fluorescence plots for singlet Jurkat cells.

FIG. 8: inv-hly mediated therapeutic protein transfer. Primary human activated T cells were infected with E. coli Top10 carrying an invasive plasmid alone (pGB3) or a modified invasive plasmid that also encoded the therapeutic protein OspF (pGB4). After infection, cells were incubated in the presence of antibiotics for up to 48 h. Cells were analysed on a flow cytometer to determine percentages of phosphorylated transcription factor ERK (p-Erk) from the total ERK (t-Erk) pool. a) Gating strategy for the t-Erk gate based on the live T cell population. b) Validation of the t-Erk gating strategy based on the total live event population. c) p-Erk percentages of t-Erk over time. *P<0.05, **P<0.005, ***P<0.0005****P<0.0001. Two-way analysis of variance (ANOVA) with Tukey's multiple comparisons test. Error bars show SEM. d) Average values of t-Erk % of all T cells and p-Erk % of all t-Erk+ cells. N=3.

FIG. 9: Process of gp140 mediated bacterial invasion of immune cells. In step 1, the gp120 protein part of the gp140 complex binds to the CD4 receptor on a target T cell. Binding to CD4 changes gp140 conformation and exposes the binding sites of gp41. Upon exposure gp41 binds to the co-receptor CCR5 which pulls the bacterial outer membrane and the T cell membrane closer together. In step 2, close proximity of both membranes leads to membrane fusion. In step 3, therapeutic molecules located in the bacterial periplasm get released into the T cell cytoplasm to interact with intracellular targets. In step 4, the bacterial inner membrane eventually lyses due to structural integrity loss and bacterial auxotrophies, which releases cytoplasmic therapeutic DNA or other molecules into the T cell cytoplasm.

FIG. 10: Fluorescence microscopy images of bacterial gp140 surface expression. E. coli TOP10 expressing the gp140 delivery construct was labelled with FITC conjugated anti-gp160 antibody and imaged to confirm surface expression of the protein complex. Red boxes are zoomed in from images on the FITC channel.

FIG. 11: gp140 mediated periplasmic protein transfer. Primary human activated T cells were infected with E. coli Shuffle T7 carrying an invasive gp140 plasmid either together or without an mTurquoise reporter plasmid. Following culture for 6 hours in the presence of antibiotics, cells were analysed on a flow cytometer (a&b) and fluorescence microscopy (c) for mTurquoise2 expression. a&b) data points show individual values for 3 replicates per condition. c) White arrow heads indicate adherent bacteria that lacked mTurquoise2 expression. The white circle indicates a non-adherent bacterial cells that lacked mTurquise2 expression. mTurquoise was pseudocolored to cyan. KO525-A+=mTurquoise detection filter.

FIG. 12: Gating strategy for gp140 mediated secreted protein transfer to primary lymphocytes. Plots are shown for the uninfected cell control (NC) at 2 h p.i.

FIG. 13: Percentages of live, lymphocytes, singlets, and CD3+ subtypes for for gp140 mediated secreted protein transfer to primary lymphocytes.

FIG. 14: Lymphocyte gating strategy for gp140 mediated secreted protein transfer to primary lymphocytes.

FIG. 15: gp140 mediated secreted protein transfer. Primary human lymphocytes were isolated from buffy coats and infected with E. coli Shuffle T7 carrying an invasive gp140 plasmid either together (gp140+TEM-1) or without (gp140) a β-lactamase reporter plasmid. After infection, cells were incubated in the presence of antibiotics followed by loading with CCF4-AM. Loaded cells were analysed on a flow cytometer to determine percentages of blue cells, indicative of protein transfer. a) Percentage of blue cells from CD3+ cells. b) Percentage of blue cells from all CD3− cells. c) Cell subtype percentages of blue CD3+ cells. d) Cell subtype percentages of blue CD3− cells. e) Comparison of cell subtype percentages between blue CD3+ cells and all CD3+ cells at 2 h p.i. f) Comparison of cell subtype percentages between blue CD3+ cells and all CD3+ cells at 4 h p.i. g) Comparison of cell subtype percentages between blue CD3− cells and all CD3− cells at 2 h p.i. h) Comparison of cell subtype percentages between blue CD3− cells and all CD3− cells at 4 h p.i. *P<0.05, **P<0.005, ***P<0.0005****P<0.0001. two-way analysis of variance (ANOVA) with Tukey's multiple comparisons test for comparisons with more than 2 variables (a&b) or Šidák's multiple comparisons test for comparisons with less than 3 variables (c-h). Error bars show SEM. N=3.

FIG. 16: gp140 mediated secreted protein transfer. Primary human activated T cells were infected with E. coli Shuffle T7 carrying an invasive gp140 plasmid either together (gp140+TEM-1) or without (gp140) a β-lactamase reporter plasmid. After infection, cells were incubated in the presence of antibiotics followed by loading with CCF4-AM. Loaded cells were analysed on a flow cytometer to determine percentages of blue cells, indicative of protein transfer. a) Percentage of identified T cells from all events. b) Percentage of blue cells from all cells. *P<0.05, **P<0.005, ****P<0.0001; two-way analysis of variance (ANOVA) with Tukey's multiple comparisons test. Error bars show SEM. N=3.

FIG. 17: gp140 mediated secreted protein transfer. Primary human lymphocytes were isolated from buffy coats and infected with E. coli Shuffle T7 carrying an invasive gp140 plasmid either together (gp140+TEM-1) or without (gp140) a β-lactamase reporter plasmid. After infection, cells were incubated in the presence of antibiotics followed by loading with CCF4-AM. Loaded cells were analysed on a flow cytometer to determine percentages of blue cells, indicative of protein transfer. a) Percentage of blue cells from all lymphocytes. b) CD3+ and CD3− percentages of blue lymphocytes at 2 h p.i. c) CD3+ and CD3− percentages of blue lymphocytes at 4 h p.i. *P<0.05, **P<0.005, ***P<0.0005****P<0.0001. two-way analysis of variance (ANOVA) with Tukey's multiple comparisons test. Error bars show SEM. N=3.

FIG. 18: Comparison of injection routes. Healthy CB6F1 mice were injected either intravenously (i.v.) or intraperitoneal (i.p.) with 1×108 cfu/injection of the auxotrophic and invasive EcNΔdapA+pSQ11. a) CFUs of bacteria recovered from tail vein blood samples at indicated timepoints. b) Weight of major organs after 1 week. c) Total body weight or mice before (Od) and 1 week after (7d) injection. *P<0.05; two-way analysis of variance (ANOVA) with Tukey's multiple comparisons test for comparisons with more than 2 variables (a&b) or Šidák's multiple comparisons test for comparisons with less than 3 variables (c-h). Error bars show SEM. N=3.

FIG. 19: Maximum tolerated injection dose in rodents. Healthy CB6F1 mcie (a&b) or Sprague Dawley rats (c&d) were injected with different concentrations of auxotrophic and invasive EcNΔdapA+pSQ11. a&c) Total body weight per animal for different injection doses. b&d) Body temperature per animal for different injection doses. *P<0.05; two-way analysis of variance (ANOVA) with Tukey's multiple comparisons test. Error bars show SEM. N=2. Cross indicates dead animal. ND=no data.

FIG. 20: Immunofluorescence microscopy images of Hela cells following infection with cells of engineered EcN invasive strain (harboring pSQ11) and harboring a mammalian mCherry reporter plasmid (PL0017) at a MOI of 50. Arrows indicate fluorescent cells. Split arrows indicate cell replication events. Times above the images indicate days (d), hours (h), and minutes (m) post infection.

### DETAILED DESCRIPTION

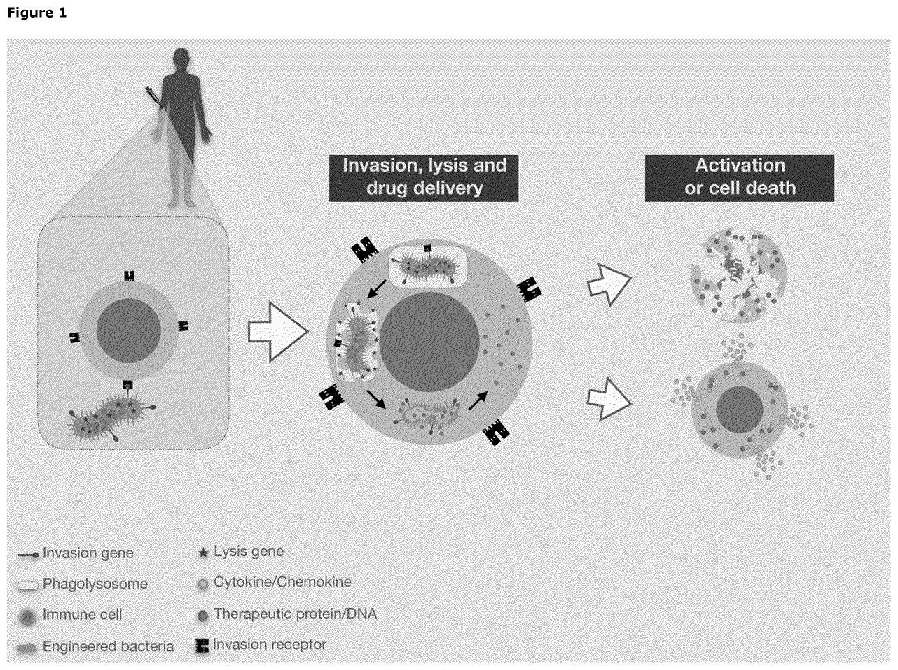

The invasive recombinant bacterial cell of the invention comprises one or more recombinant nucleic acid molecules encoding one or more therapeutic agent(s) for use in the prevention and/or treatment of an immune-related disorder in a subject in need thereof. The recombinant bacterial cell functions as a bacteria-mediated delivery vector for in vivo or ex-vivo delivery of said one or more recombinant nucleic acid molecules or said one or more therapeutic agent(s) encoded by said one or more recombinant nucleic acid molecules to a mammalian non-phagocytic immune cell for use in prevention and/or treatment of an immune-related disorder in a subject. FIG. 1 represents an illustration of the invention. The non-phagocytic immune cell and the subject at risk of developing and/or diagnosed as having an immune-related disorder is mammalian, such as a primate, bovine, ovine, porcine, feline, buffalo, canine, goat, equine, donkey, and camel, in particular a human primate.

I. A Bacteria-Mediated Delivery Vector

In one aspect, the invasive recombinant bacterial cell is engineered to express one or more genes that enable the cell to both invade and release its therapeutic payload in a non-phagocytic immune cell. Expression of the one or more genes enables the recombinant bacterial cell to both invade the non-phagocytic immune cell, where it is typically internalized in primary vesicles (such as phagosomes); and to then escape into the cytosol due to induced permeabilization of the primary vesicles. Once the recombinant bacterial cell has escaped, it is genetically adapted to undergo lysis and thereby release its therapeutic payload into the cytosol, such that the therapeutic payload can bring about a therapeutic effect on, or by means of, said non-phagocytic immune cell. Examples of recombinant bacterial cells engineered for this purpose are detailed below:

In one example, the invasive recombinant bacterial cell of the invention comprises genes encoding a first protein belonging to the 1.B.54 family of Intimin/Invasin (Int/Inv) or Autotransporter-3 (AT-3) proteins; and a second protein belonging to the 1.C.12.1.7 family of thiol-activated cholesterol-dependent cytolysin (cdc) proteins, where the expression of said first and second proteins confers on the cell the ability to act as a bacteria-mediated delivery vector for delivery of the therapeutic agent to a mammalian non-phagocytic immune cell.

The first protein, belonging to the 1.B.54 family of homologous proteins, is an outer membrane (OM) protein found in strains of Yersinia spp. (Inv), pathogenic E. coli (Int), and Citrobacter spp (Int) [Example 1]. Expression of the first protein by the recombinant bacterial cell mediates its attachment to, and invasion of, the mammalian non-phagocytic immune cell. A suitable first protein includes an invasin belonging to the 1.B.54.1.2 sub-family, such as an invasin derivable from pathogen Yersinia pseudotuberculosis.

Expression of Y. pseudotuberculosis invasin by the recombinant bacterial cell, as shown herein [Example 3], both mediates invasion of an immune cell, and its subsequent uptake into the immune cell's phagolysosome. While not bound by theory, said invasion may occur when the outer membrane invasin binds to an integrin on the surface of the target non-phagocytic immune cell, such as an integrin belonging to one or more of subtypes α3β1, α4β1, α5β1, and α6β1 integrin.

In one aspect, when the first protein is an invasin, the primary amino acid sequence of said protein may be one having at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 2. Alternatively, the amino acid sequence of said protein is modified, by the substitution of its signal peptide sequence with the sequence of a signal peptide native to the recombinant bacterium in which the first protein is expressed [Example 3]. For example, where the recombinant bacterium is a strain of E. coli, then the primary amino acid sequence of the invasin comprising a substitute E. coli signal peptide is one having at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 4.

The second protein, belonging to the 1.C.12.1.7 family of homologous proteins, is a cytolysin found in strains of Listeria monocytogenes. Following invasion of a non-phagocytic immune cell and its inclusion in a primary vesicle (e.g. phagocyte), the cytolysin, expressed and secreted by the recombinant bacterial cell, causes the formation of pores in the primary vesicle membrane of the immune cell, allowing escape of the bacterial cell into the cytosol. A suitable second protein includes a Listeriolysin O derivable from Listeria monocytogenes serovar 1/2a. Expression of a combination of the L. monocytogenes Listeriolysin O and the Y. pseudotuberculosis invasin by the recombinant bacterial cell, as shown herein [Example 3], both mediates invasion of an immune cell, and its subsequent release from the immune cell's phagolysosome. While not bound by theory, the acidic conditions within the phagolysosome may induce folding and activation of pore-forming properties of the Listeriolysin O when expressed by the recombinant bacterium, while once released into the cytoplasm it is inactivated by the neutral pH.

In one aspect, when the second protein is an Listeriolysin O, the amino acid sequence of said protein may be one having at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 6. Alternatively, the amino acid sequence of said protein is modified, by the substitution of its signal peptide sequence with the sequence of a signal peptide native to the recombinant bacterium in which the second protein is expressed [Example 3]. For example, where the recombinant bacterium is a strain of E. coli, then the primary amino acid sequence of the Listeriolysin O comprising a substitute E. coli signal peptide is one having at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 8.

In a second example, the invasive recombinant bacterial cell of the invention comprises gene(s) encoding a first and second protein derived from a viral envelope glycoprotein complex, such as from HIV envelope glycoprotein complex proteins: gp120 and gp41 found in Human Immunodeficiency Virus 1 (HIV-1); in combination with and a third protein derived from a member of the 1.B.12.8.2 autotransporter-1 (at-1) family. These first, second and third proteins, that may be expressed as domains linked together in a fusion protein, confer on the cell the ability to act as a bacteria-mediated delivery vector for delivery of the therapeutic agent to a mammalian non-phagocytic immune cell. The recombinant bacterial cell may additionally express a fourth protein belonging to the 1.C.12.1.7 family of homologous proteins, preferably a cytolysin found in strains of Listeria monocytogenes, as described above in said first example, to ensure efficient intracellular delivery of bacterial therapeutics by phagolysosome lysis.

Suitable first and second proteins are gp120 and the gp41 ectodomain, derivable from the Transmitter/Founder (T/F) R5 strain BG505, that are modified by amino acid substitutions of all N glycosylation motifs (NXT/S), such that they are expressed as non-glycosylated proteins in the recombinant bacterial cell [Example 11]. When expressed as part of a fusion protein, the gp120 and gp41 protein domains may be connected by an amino acid linker. While not bound by theory, an envelope complex (e.g. spike protein trimer) comprising gp120 and gp41 proteins derived from (T/F) R5 strain BG505 will preferentially bind to a CD4 and a CCR5 receptor found on CD4+ CCR5+ T-cells. The gp41 ectodomain facilitates invasion by insertion of its hydrophobic N terminus into the immune cell membrane. Due to the affinity of the gp41 α-helices for both the bacterial- and immune-cell membranes, the respective membranes are pulled into sufficient juxtaposition to bring about their fusion.

The third protein, comprises protein domains derived from a member of the 1.B.12.8.2 family of homologous proteins, which facilitate anchoring of the gp120/gp41 envelope complex to the outer membrane of the recombinant bacterial cell. A suitable third protein may be derived from the signal peptide (SP) and the C-terminal portion of an autotransporter antigen 43 (FLU) protein from E. coli K12, the latter comprising an autochaperone (AC1) domain followed by a β-chain translocator domain.

When expressed as a fusion protein, the first and second gp120 and gp41 proteins are expressed as passenger domains fused between the SP and the AC1 domain of the third protein. The extended sequence of the SP ensures an export rate sufficient to sustain a secretion competent state of the passenger envelope complex. The C-terminal translocator domain anchors the fusion protein to the bacterial membrane by formation of a β-barrel outer membrane pore.

In one aspect, the amino acid sequence of said fusion protein (gp120+gp41) may be one having at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 10.

In one aspect, the invention provides a recombinant bacterial cell comprising recombinant genes encoding a fusion protein comprising an N-terminal signal peptide of an autotransporter antigen 43 (FLU) protein, an HIV-1 glycoprotein 120, a first linker peptide, an HIV-1 glycoprotein 41, and a second linker, an autochaperone (AC1) domain and a β-chain translocator domain of said autotransporter antigen, fused in consecutive order, such as provided in example 11.

In one embodiment, said N-terminal signal peptide of the autotransporter antigen 43 (FLU) protein may be encoded by a nucleic acid sequence having at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 171. In one embodiment, said N-terminal signal peptide of the autotransporter antigen 43 (FLU) protein may be having at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 288.

In one embodiment, said HIV-1 glycoprotein 120, said first linker peptide, and said HIV-1 glycoprotein 41 fused in consecutive order may have at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 286.

In one embodiment, said second linker may be derived from an autotransporter antigen 43 (FLU) protein from E. coli K12 having at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 179.

In one embodiment, said autochaperone (AC1) domain may be derived from an autotransporter antigen 43 (FLU) protein from E. coli K12 having at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 181.

In one embodiment, said β-chain translocator domain may be derived from an autotransporter antigen 43 (FLU) protein from E. coli K12 having at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 183.

In one embodiment, said second linker, autochaperone (AC1) domain and β-chain translocator domain fused in consecutive order may have at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 290.

In a preferred embodiment, the amino acid sequence of said fusion protein comprising an N-terminal signal peptide of an autotransporter antigen 43 (FLU) protein, an HIV-1 glycoprotein 120, a first linker peptide, an HIV-1 glycoprotein 41, and a second linker, an autochaperone (AC1) domain and a β-chain translocator domain of said autotransporter antigen may have at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 284. In one embodiment, said fusion protein is SEQ ID No.: 284.

In one embodiment, said recombinant genes encoding said fusion protein are located on a plasmid, such as pCOLA gp120-gp41-flu (SEQ ID NO 169).

In a third example, the invasive recombinant bacterial cell of the invention comprises genes encoding a first and second protein derived from components of the 1.C.36.3.1 Type III secretion system (T3SS), and membrane-anchoring protein(s), where the expression of said proteins confers the cell with the ability to act as a bacteria-mediated delivery vector for delivery of the therapeutic agent to a mammalian non-phagocytic immune cell.

Suitable first and second proteins are the invasin IpaB and IpaC proteins derivable from a pathogenic bacterium such as Shigella flexneri, together with a third protein, functioning as a membrane anchoring domain [Example 2]. While not bound by theory, IpaB initiates invasion, by forming a needle tip complex and binding to the host hyaluronan receptor CD44 and α5β1 integrin of an immune cell. IpaC, when complexed with ipaB, comprises domains for secretion; actin polymerisation at, and integration into, the immune cell membrane; resulting in internalization of the recombinant bacterial cell via filopodia and phagolysosome engulfment. IpaB further facilitates escape of the bacterium from the phagolysosomal by formation of ion pores. The third protein may either be invasin IpaD or alternatively a GPI-anchored protein such as a member of the bacterial ice nucleation family (e.g. INA-K and INA-Q) derivable from Pseudomonas spp that is fused to each of the IpaB and IpaC proteins. When the recombinant bacterial cell expresses each of IpaB and IpaC as fusion proteins, fused to the C-terminus of a truncated INA protein, its glucosylphosphatidylinositol (GPI) anchor domain tethers each fusion protein to the outer cell membrane, while the INA repeat region allows complex formation between the extracellularly displayed IpaB and IpaC.

In one aspect, the amino acid sequence of the fusion proteins INA.K-IpaB-IpaC may be one having at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 12.

In one aspect, the amino acid sequence of the proteins IpaB, IpaC and IpaD may be one having at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 14, 16, and 18 respectively.

In a further aspect, the invasive recombinant bacterial cell of the invention is additionally genetically adapted to undergo lysis following its released from the primary vesicle (e.g. phagosome) allowing release of its therapeutic payload. For example, the bacterial cell is modified to inactivate the chromosomal DapA gene, such that the cells undergo lysis in the immune cell due to a failure to express 4-Hydroxy-tetrahydrodipicolinate synthase (DAP), essential for cell wall synthesis (Example 1).

In a further aspect, the invasive recombinant bacterial cell of the invention is a live bacterium and a species of a genus selected from among Escherichia, Bacteroides, Akkermansia, Alistipes, Prevotella, Parabacteroides, Odoribacter, Enterobacter, Klebsiella, Citrobacter, Shigella, Listeria, Yersinia, Citrobacter, Bartonella, Agrobacterium, Salmonella, Helicobacter, Bartonella, Anaplasma, Ehrlichia, Coxiella, Chlamydia, Rickettisa, Legionella, Mycobacterium, Brucella, and Pseudobutyrivibrio. In a further embodiment, the invasive recombinant bacterial cell is selected from the genus Lactobacillus or Bifidobacterium. For example, the recombinant gram-negative bacterium is E. coli, since members of this species have the added advantage of being easily engineered, and in particular it is E. coli Nissle since this is a well-characterized probiotic that is classified as a risk group I organism.

In a further aspect, the invasive recombinant bacterial cell of the invention comprises on one or more plasmids that comprise the one or more recombinant nucleic acid molecules encoding the therapeutic agent. The coding sequence for the therapeutic agent in each of the one or more recombinant nucleic acid molecules is operatively linked to a promoter, RBS, signal peptide region, terminator, and polyadenylation signal functional in either a prokaryotic or a eukaryotic cell, these being selected to provide a desired expression level and location in the recombinant bacterium of the invention or in the invaded non-phagocytic immune cell respectively.

In a further aspect, the one or more recombinant nucleic acid molecules encoding a therapeutic agent additionally comprises at least one DNA nuclear targeting sequence(s) (DTS) to facilitate efficient import into the nucleus of the non-phagocytic immune cell, in particular into non-dividing immune cells. The inclusion of DTSs increases transcription rates of the one or more nucleic acid molecules transferred into an invaded non-phagocytic immune cell. The DTS(s) are present as multiple direct repeat sequences, preferably located before the promoter sequence and after the poly A signal of the therapeutic agent coding sequence. Recombinant bacterial cells of the invention, whose one or more recombinant nucleic acid molecules comprise DTSs selected from among an SV40 enhancer (SEQ ID No.: 19); a glucocorticoid receptor binding site (SEQ ID No.: 20); and a NF-κB-binding site (SEQ ID No.: 21), are illustrated in example 4. Relative levels of nuclear import and subsequent expression of the recombinant nucleic acid molecules comprising DTSs may be detected by measuring signal strength of a co-expressed fluorescent reporter gene and the number of fluorescent cells. Suitable DTSs and their optimal number of repeats are given in Table 1; further indicating their genetic sources.

| | Optimal | Nucleotide sequence |
|---|---|---|
| DTS derived from genes encoding: | No. repeats | of DTS |
| tumor suppressor protein p53 | 14× | TGCCTGGACTTGCCTGG |
| | | [SEQ ID No.: 22] |
| the activator protein 1 (AP-1) | 7× | TGACTAA [SEQ ID No.: |
| | | 23] |
| CCAAT-enhancer binding protein | 3× | ATTGCGCAAT |
| (C/EBP) | | [SEQ ID No.: 24] |
| consensus sites for cyclic AMP | 4× | AGCCTGACGTCAGAG |
| response element (CRE) | | [SEQ ID No.: 25] |
| retinoid X receptor response element | 5× | AGGTCAN |
| (DR1/RXRE) | | [SEQ ID No.: 26] |
| vitamin D receptor response element | 4× | AGGTCANNN |
| (DR3/VDR) | | [SEQ ID No.: 27] |
| active thyroid hormone receptor | 2× | CAGGAGGTCA |
| response element (DR4/TR) | | [SEQ ID No.: 28] |
| retinoic acid receptor response | 5× | AGGTCANNNNN |
| element (DR5/RARE) | | [SEQ ID No.: 29] |
| early growth response factor 1 | 3× | GGGGTGGGGN |
| (Egr-1) | | [SEQ ID No.: 30] |
| interferon γ-activated sequence (GAS) | 4× | AGTTTCATATTACTCTAAATC |
| | | [SEQ ID No.: 31] |
| glucocorticoid response element (GRE) | 4× | GGTACATTTTGTTCT |
| | | (Second repeat in |
| | | antisense) |
| | | [SEQ ID No.: 20] |
| interferon-stimulated response | 5× | TAGTTTCACTTTCCC |
| element (ISRE) | | [SEQ ID No.: 32] |
| LPS IL-1β response element (LILRE) | 4× | TCACTTCCTGAGAG |
| | | [SEQ ID No.: 33] |
| nuclear factor of activated T cells | 4× | GGAGGAAAAACTGTTTCATACA |
| (NFAT) | | GAAGGCGT [SEQ ID No.: |
| | | 34] |
| binding sites for nuclear factor κB | 5× | TGGGGACTTTCCGC |
| (NF-κB) | | [SEQ ID No.: 21] |
| serum response element (SRE) | 5× | AGGATGTCCATATTAGGACATC |
| | | T [SEQ ID No.: 35] |
| serum response factor (SRF) | 5× | GTCCATATTAGGAC |
| | | [SEQ ID No.: 36] |
| TGF-β/Activin response element (TARE) | 3× | CATTGTCAGTCTAGACATACTC |
| | | CGAGATTGTGGATTGAGA |
| | | [SEQ ID No.: 37] |
| SV40 (simian virus 40) ehancer | 2× | GGTGTGGAAAGTCCCCAGGCTC |
| | | CCCAGCAGGCAGAAGTATGCAA |
| | | AGCATGCATCTCAATTAGTCAG |
| | | CAACCA [SEQ ID No.: 19] |

In a further aspect, the therapeutic agent encoded by the one or more recombinant nucleic acid molecules is a therapeutic protein comprising nuclear localization sequences (NLS) fused to both the C- and N-terminal end of the therapeutic protein. Suitable NLS are given in Table 2; further indicating their genetic sources.

TABLE 2
Nuclear localization sequences (NLS)

| NLS derived from primary | |
|---|---|
| sequences of: | AA sequence |
| SV40 (simian virus 40) large T antigen | PKKKRKV [SEQ ID No.: 38] |
| c-Myc | PAAKRVKLD [SEQ ID No.: 39] |
| nucleoplasmin | KRPAATKKAGQAKKKK [SEQ ID No.: 40] |
| Human T-cell leukemia virus type-1 | MPKTRRRPRRSQRKRPPT |
| Rex protein (HTLV-1 Rex) | [SEQ ID No.: 41] |
| EGL-13 | MSRRRKANPTKLSENAKKLAKEVEN |
| | [SEQ ID No.: 42] |
| TUS-protein | KLKIKRPVK [SEQ ID No.: 43] |
| M9 domain of hnRNP A1 | NDFGNYNNQSSNFGPMKGGNFGGRSSGPY |
| | [SEQ ID No.: 44] |
| Epstein-Barr virus nuclear antigen 1 | KRPRSPSS [SEQ ID No.: 45] |
| (EBNA-1) | |
| Mitogen-activated protein kinase 1/3 | LDQLNHILGILGSPSQEDL [SEQ ID No.: |
| (ERK 1/2) | 46] |
| Nuclear factor of activated T-cells, | RSSRPASPCNKRKYS [SEQ ID No.: 47] |
| cytoplasmic 1 (NFATc1) | |
| Parathyroid hormone-related protein | RYLTQETNKVETYKEQPLKTPGKKKKGKP |
| (PTHrP) | [SEQ ID No.: 48] |

II. Therapeutic Payload of the Bacteria-Mediated Delivery Vector

The invasive recombinant bacterial cell of the invention comprises one or more recombinant nucleic acid molecule(s) encoding one or more therapeutic agent(s) for use in prevention and/or treatment of an immune-related disorder in a subject in need thereof, wherein said agent is one or more recombinant nucleic acid molecules (RNA or DNA), or one or more proteins, or a combination thereof. In one embodiment, a wide range of therapeutic agents can be delivered, e.g. a Chimeric Antigen Receptor protein; a small interfering RNA; a protein inhibitor of any one of T cell activation, T cell suppression, T cell proliferation and T cell cell death; a protein inducer of any one of T cell activation, T cell suppression, T cell proliferation and T cell cell death; a cytotoxin; a cytokine; a chemokine, and a CRISPR-Cas9; as is further illustrated below.

In another embodiment, the therapeutic agent is a recombinant or native DNA, RNA, or protein agent selected from the group: a Chimeric Antigen Receptor, a small interfering RNA, a protein inhibitor of any one of T cell activation; T cell suppression; T cell differentiation; T cell maturation; T cell proliferation and T cell cell death; a protein s inducer of any one of T cell activation; T cell suppression; T cell differentiation; T cell maturation; T cell proliferation and T cell cell death; an oxidoreductase or an inhibitor or an activator thereof; a transferase or an inhibitor or an activator thereof; a hydrolase or an inhibitor or an activator thereof; a lyase or an inhibitor or an activator thereof; an isomerase or an inhibitor or an activator thereof; a ligase or an inhibitor or an activator thereof; a translocase or an inhibitor or an activator thereof; a cytotoxin; a cytokine; a nanobody; a monobody; an affibody; an antibody fragment; a DARPin; a nanoparticle; a growth factor; a hormone; a chemokine, and; a CRISPR-Cas system.

II.i Nucleic Acid Therapeutic Agents:

In one aspect the therapeutic payload delivered by the recombinant bacterial cell of the invention to the non-phagocytic immune cell comprises one or more recombinant nucleic acid molecules. The therapeutic effect of the payload on the immune-related disorder is mediated by the expression of proteins encoded by said one or more recombinant nucleic acid molecules in the immune cell following delivery of the payload. Expression of the encoded proteins in the immune cell is facilitated by eukaryotic expression sequences (promoter, RBS, and polyadenylation signal) operatively linked to the protein coding sequences in the recombinant nucleic acid molecules.

A first example of a protein expressed in an immune cell on delivery of the payload is a Chimeric Antigen Receptor (CAR), which is designed to confer a T cell with the ability to recognize and bind to specific epitopes of a causal agent of a given disorder.

When the causal agent is an infectious disease, the CAR may comprise an antigen recognition domain (e.g. single-chain variable fragment; receptor domain) recognizing an epitope specific to the infectious agent, such that the resulting CAR-T cells have a therapeutic effect on an infectious disease (as exemplified in Table 3).

TABLE 3
Infectious diseases treated by bacteria-mediated
delivery vector payloads encoding CARs *

| | Epitopes | CAR antigen |
|---|---|---|
| | recognized | recognition |
| Infectious disease | by CAR | domain |
| Human | CD4 binding site on gp-120 | CD4 |
| Immunodeficiency | Env/gp120 glycans | CD4/CD28 |
| Virus (HIV) | V1/V2 glycan loop | PGT145-scFv |
| | CD4-induced epitope | CD8 |
| | on gp120/CD4 binding | |
| | site | |
| Hepatitis B Virus (HBV) | S HBV surface protein | C8-scFv |
| | HBV surface antigen | 19.79.6-scFv |
| Hepatitis C Virus (HCV) | E2 glycoprotein | e137-scFv |
| Cytomegalovirus (CMV) | Glycoprotein B | 27-287-scFv |
| | Virally encoded FcRs | IgG1 or IgG4 Fc |
| | | mutated |
| Aspergillus fumigatus | β-glucan | Dectin 1 |

* Seif, Einsele, & Löffler, (2019)

When the causal agent is cancer, the CAR may comprise an antigen recognition domain (e.g. single-chain variable fragment; receptor domain) recognizing an epitope specific for the cancer cell, such that the resulting CAR-T cells have a therapeutic effect on the cancer. The recognized epitope on a cancer cell includes surface receptors, and by way of example a receptor may be selected from among: CD19, BCMA, CD22, CD20, CD123, CD30, CD38, CD33, CD138, CD56, CD7, CLL-1, CD10, CD34, CS1, CD16, CD4, CD5, IL-1-RAP, ITGB7, k-IgG, TACI, TRBC1, MUC1, NKG2D, PD-L1, CD133, CD117, LeY, CD70, ROR1, AFP, AXL, CD80, CD86, DLL3, DR5, FAP, FBP, LMP1, MAGE-A1, MAGE-A4, MG7, MUC16, PMEL, ROR2, VEGFR2, CD171, CLD18, EphA2, ErbB, Fra, PSCa, cMet, IL13Ra2, EPCAM, EGFR, PSMA, EGFRcIII, GPC3, CEA, HER2, GD2 and Mesothelin (MacKay et al., 2020).

II.ii RNA Interference Mediated Therapeutics:

In one aspect the therapeutic effect of the payload delivered by the recombinant bacterial cell of the invention to the non-phagocytic immune cell is mediated by small interfering RNA (siRNA) encoded by short hairpin RNA (shRNA) coding sequences comprised in said one or more recombinant nucleic acid molecules. Transcription of the siRNA in the immune cell nucleus may be facilitated by eukaryotic expression sequences (promoter and polyadenylation signal, e.g. U6 promoter and a SV40 poly A termination signal) operatively linked to the shRNA coding sequences in the recombinant nucleic acid molecules [example 6]. Alternatively, the therapeutic payload comprises one or more siRNA molecules transcribed from the one or more recombinant nucleic acid molecules in the recombinant bacterial cell, whose transcription is facilitated by prokaryotic expression sequences (promoter, RBS, and terminator, e.g. T7 prokaryotic expression sequences) operatively linked to the shRNA coding sequences in the recombinant nucleic acid molecules [example 6]. On delivery to the immune cell cytoplasm, target messenger RNA (mRNA) complementary to the siRNA and detected by RNA-induced silencing complex (RISC) is then cleaved by a nuclease under direction of the RISC.

Disorders targeted by siRNA mediated therapy include silencing: inhibitors of T-cell activation in cancer; signaling pathways that activate T-cells in inflammation; genes required for viral invasion during HIV infection (Freeley & Long, 2013); CD4 in T-cells during autoimmune disease (Lee et al., 2012); ZAP-70 to reduce T-cell activation in delayed type hypersensitivity (Gust et al., 2008); SOCS3 to reduce allergic airway response in asthma and to reduce insulin resistance in diabetes (Jorgensen et al., 2013; Moriwaki et al., 2011); Cblb to improve efficacy of tumor vaccines in melanoma (Hinterleitner et al., 2012); JAK3 in CD3+ T-cell blasts to suppress Th1-mediated inflammatory responses (Gómez-Valadés et al., 2012); SOCS-1 in CD8 T-cells to improve anti-tumor response (Dudda et al., 2013); STAT3 to decrease graft-vs-host response and increase anti-tumor response in CD4 T-cells (Pallandre et al., 2007); CD4 to prohibit HIV entry into T-cells (Novina et al., 2002); CCR5 to prohibit HIV entry into T-cells (Kumar et al., 2008), NR4A2 to decrease inflammation in multiple sclerosis (Doi et al., 2008), FOXP3 or IL10 in CCR4+ Treg cells to inhibit breast cancer metastatis into the lung (Biragyn et al., 2013); T-bet in autoreactive encephalitogenic T-cells to inhibit development of multiple sclerosis (Lovett-Racke et al., 2004); GATA-3 in TH1 cells to enhance cancer vaccine response in colorectal cancer (Tesniere et al., 2010); and CD3 to treat acute allograft rejection and graft-vs-host disease.

II.iii Protein Therapeutic Agents:

In one aspect the therapeutic payload delivered by the recombinant bacterial cell of the invention to the non-phagocytic immune cell comprises one or more proteins. The proteins are encoded by the one or more recombinant nucleic acid molecules in the recombinant bacterial cell and expressed prior to their delivery to the immune cell. Expression of the encoded proteins in the bacterial cell is facilitated by prokaryotic expression sequences (promoter, RBS, signal peptide sequence, and terminator) operatively linked to the protein coding sequences in the recombinant nucleic acid molecules.

In one example, the protein is a transcription factor that mediates a therapeutic effect on an immune-related disorder by modulating cell differentiation, activation, or proliferation on delivery to a non-phagocytic immune cell (e.g. lymphocyte) associated with a given disorder.

In one example, the protein is an inhibitor of a transcription factor that is a causal agent of pro-inflammatory downstream signaling in diseases such as lupus, rheumatoid arthritis, and type 1 diabetes.

For instance, protein therapeutic agents delivered by the recombinant bacterial cells of the invention may be selected from among: Shigella virulence factors (Mattock & Blocker, 2017); OspC3 inhibiting Caspase-4-Mediated Inflammatory Cell Death; OspF inhibiting phosphorylation of ERK1/2 [Example 8]; OspG inhibiting NFκB activation; OspI inhibiting NFκB Activation; OspZ inhibiting NFκB activation; IpaH9.8, inhibiting NFκB response; IpaH0722 inhibiting NFκB activation; IpgD activating Akt/PI3K signaling pathway; Yersinia pestis effector YopH; a phosphotyrosine phosphatase that dephosphorylates phosphotyrosine and the T-cell scaffold proteins LAT and SLP-76, which inhibits TCR signaling and T-cell activation and proliferation (Wei et al., 2012); T3SS effectors NleE and NleB, where NleE inhibits NFκB through blockage of p65 nuclear transport upon TNFα and IL-1β stimulation and further suppresses NFkB by inhibiting activation of IKKβ and therefore degradation of IκB (Nadler et al., 2010), and NleB enhances NFkB inhibition of NleE and inhibits FAS death receptor mediated extrinsic apoptosis signaling, thereby protecting affected mammalian target cells from apoptotic cell death (Pollock et al., 2017) [Example 9].

Alternatively, the protein delivered to the non-phagocytic immune cell is selected from among adenosine deaminase (ADA) for the treatment of severe combined immunodeficiency (SCID) (Flinn & Gennery, 2018); L-asparaginase for the treatment of acute lymphoblastic leukemia (Müller & Boos, 1998) (see example 10); azurin in the treatment of cancerous lymphocytes (Punj et al., 2004); cytotoxins such as colibactin, glidobactin, and luminmide in the treatment of cancerous lymphocytes (R. Li et al., 2019); pro-inflammatory cytokines/chemokines in the treatment of immunodeficiency; and anti-inflammatory cytokines/chemokines in the treatment of inflammatory diseases (Luheshi, Rothwell, & Brough, 2009).

II.iv Combinations of Protein, DNA, and RNA Therapeutic Agents:

In one aspect the therapeutic payload delivered by the recombinant bacterial cell of the invention to the non-phagocytic immune cell comprises one or more Cas endonucleases and one or more guide RNA molecules (CRISPR Cas); e.g. Cas9 and single guide RNAs (gRNA), for use in the therapeutic prevention and/or treatment of an immune-related disorder. The CRISPR Cas is encoded by the one or more recombinant nucleic acid molecules in the recombinant bacterial cell and expressed prior to its delivery to the immune cell. Expression of the encoded CRISPR Cas in the bacterial cell is facilitated by prokaryotic expression sequences (promoter, RBS, and terminator) operatively linked to their respective coding sequences in the recombinant nucleic acid molecules. An N-terminal nuclear localization signal (e.g. SV40 NLS) may be fused to the Cas endonuclease (Cas9) to improve nuclear transport of the delivered protein. Alternatively, the recombinant nucleic acid molecules encoding both the Cas endonuclease and the gRNA are delivered to the invaded mammalian non-phagocytic immune cell where they are expressed under the control of mammalian expression sequences (promoter, enhancer, and poly A tail) operatively linked to their respective coding sequences in the recombinant nucleic acid molecules. An N-terminal DTS signal may be fused to the DNA sequences encoding the Cas nuclease and the gRNA to improve nuclear localization and enhanced expression.

CRISPR Cas, delivered as the therapeutic payload to non-phagocytic immune cells, may be used for prevention and/or treatment of HIV by directing gRNA to the following target sequences: HIV provirus LTR (U3 region (Ebina et al., 2013), HIV provirus LTR (R region) (Liao et al., 2015), HIV provirus second exon of Rev (Zhu et al., 2015), HIV provirus Gag/Pol/Rev/Env (Wang et al., 2016), T cell co-receptor CCR5 (Qi et al., 2018), and T cell co-receptor CXCR4 (Hou et al., 2015).

CRISPR Cas, as the therapeutic payload, may be used in the treatment of various types of cancer by directing gRNA to oncogenes in cancerous lymphocytes or to human programmed death-1 PD-1 receptor in T-cells to counteract PD-L1 expression and subsequent immune suppression by cancer cells (Su et al., 2016).

CRISPR Cas, as the therapeutic payload, may be used to target a mutated gene by additionally co-delivering a homologous replacement DNA sequence for restoring the non-mutant gene such as: targeting loss of function mutations in the Adenosine deaminase (ADA) ada gene in ADA deficiency (Flinn & Gennery, 2018), targeting mutations in the IL2RG gene in X-linked severe combined immunodeficiency (X-SCID) (Allenspach, Rawlings, & Scharenberg, 1993).

II.v Immune-Related Disorders:

Those disorders for which the invasive recombinant bacterial cells of the invention may be used as a bacteria-mediated delivery vector for use in providing therapeutic prevention and/or treatment include: autoimmune disorder(s); lymphoproliferative disorders; and cancer(s).

When the immune-related disorder is an autoimmune disorder it may be selected from the group: Inflammatory bowel disease; Celiac disease; Severe combined immunodeficiency (SCID); Organ transplant rejection (graft vs host disease); Asthma; Crohn's disease; Myocarditis; Postmyocardial infarction syndrome; Postpericardiotomy syndrome; Subacute bacterial endocarditis (SBE); Anti-Glomerular Basement Membrane nephritis; Lupus nephritis; Interstitial cystitis; Autoimmune hepatitis; Primary biliary cholangitis (PBC); Primary sclerosing cholangitis; Antisynthetase syndrome; Alopecia Areata; Autoimmune Angioedema; Autoimmune progesterone dermatitis; Autoimmune urticaria; Bullous pemphigoid; Cicatricial pemphigoid; Dermatitis herpetiformis; Discoid lupus erythematosus; Epidermolysis bullosa acquisita; Erythema nodosum; Gestational pemphigoid; Hidradenitis suppurativa; Lichen planus; Lichen sclerosus; Linear IgA disease (LAD); Morphea; Pemphigus vulgaris; Pityriasis lichenoides et varioliformis acuta; Mucha-Habermann disease; Psoriasis; Systemic scleroderma; Autoantibodies: Anti-nuclear antibodies, anti-centromere and anti-scl70/anti-topoisomerase antibodies; Vitiligo; Addison's disease; Autoimmune polyendocrine syndrome (APS) type 1; Autoimmune polyendocrine syndrome (APS) type 2; Autoantibodies: anti-21 hydroxylase; anti-17 hydroxylase.; Autoimmune polyendocrine syndrome (APS) type 3; Autoimmune pancreatitis; Diabetes mellitus type 1; Autoimmune thyroiditis; Ord's thyroiditis; Graves' disease; Autoimmune oophoritis; Endometriosis; Autoimmune orchitis; Sjögren syndrome; Autoimmune enteropathy; Coeliac disease; Crohn's disease; Esophageal achalasia; Ulcerative colitis; Antiphospholipid syndrome; Aplastic anemia; Autoimmune hemolytic anemia; Autoimmune lymphoproliferative syndrome; Autoimmune neutropenia; Autoimmune thrombocytopenic purpura; Cold agglutinin disease; Essential mixed cryoglobulinemia; Evans syndrome; Pernicious anemia; Pure red cell aplasia; Thrombocytopenia; Adiposis dolorosa; Adult-onset Still's disease; Ankylosing spondylitis; CREST syndrome; Drug-induced lupus; Enthesitis-related arthritis; A subtype of Juvenile Rheumatoid Arthritis.; Eosinophilic fasciitis; Felty syndrome; IgG4-related disease; Juvenile arthritis; Lyme disease; Mixed connective tissue disease; Palindromic rheumatism; Parry-Romberg syndrome; Parsonage-Turner syndrome; Psoriatic arthritis; Reactive arthritis; Relapsing polychondritis; Retroperitoneal fibrosis; Rheumatic fever; Rheumatoid arthritis; Sarcoidosis; Schnitzler syndrome; Systemic lupus erythematosus; Undifferentiated connective tissue disease; Dermatomyositis; Fibromyalgia; Inclusion body myositis; Myositis; Myasthenia gravis; Neuromyotonia; Paraneoplastic cerebellar degeneration; Polymyositis; Acute disseminated encephalomyelitis; Acute motor axonal neuropathy; Anti-N-Methyl-D-Aspartate Receptor Encephalitis; Balo concentric sclerosis; Bickerstaff's encephalitis; Chronic inflammatory demyelinating polyneuropathy; Guillain-Barre syndrome; Hashimoto's encephalopathy; Idiopathic inflammatory demyelinating diseases; Lambert-Eaton myasthenic syndrome; Multiple sclerosis; Oshtoran syndrome; Pediatric Autoimmune Neuropsychiatric Disorder Associated with Streptococcus; Progressive inflammatory neuropathy; Restless legs syndrome; Stiff-person syndrome; Sydenham's chorea; Transverse myelitis; Autoimmune retinopathy; Autoimmune uveitis; Cogan syndrome; Graves' ophthalmopathy; Intermediate uveitis; Ligneous conjunctivitis; Mooren's ulcer; Neuromyelitis optica; Opsoclonus myoclonus syndrome; Optic neuritis; Scleritis; Susac's syndrome; Sympathetic ophthalmia; Tolosa-Hunt syndrome; Autoimmune inner ear disease; Meniere's disease; Behçet's disease; Rare variant: Hughes-Stovin syndrome.; Eosinophilic granulomatosis with polyangiitis; Giant cell arteritis; Granulomatosis with polyangiitis; IgA vasculitis; Kawasaki disease; Leukocytoclastic vasculitis; Lupus vasculitis; Rheumatoid vasculitis; Microscopic polyangiitis; Polyarteritis nodosa; Polymyalgia rheumatica; Urticarial vasculitis; Vasculitis; Primary immunodeficiency; Chronic fatigue syndrome; Complex regional pain syndrome; Eosinophilic esophagitis; Gastritis; Interstitial lung disease; POEMS syndrome; Raynaud's phenomenon; Primary immunodeficiency; and Pyoderma gangrenosum. The autoimmune disorder may also be allergy.

When the immune-related disorder is cancer it may be selected from the group: Acute Lymphoblastic Leukemia; Acute Myeloid Leukemia; Adrenocortical Carcinoma; AIDS-Related Cancer; AIDS-Related Lymphoma; Lymphoma; Primary CNS Lymphoma; Anal Cancer; Gastrointestinal Carcinoid Tumor; Astrocytomas; Atypical Teratoid/Rhabdoid Tumor, Brain Cancer; Basal Cell Carcinoma; Bile Duct Cancer; Bladder Cancer; Bone Cancer; Ewing Sarcoma; Osteosarcoma; Malignant Fibrous Histiocytoma; Brain Tumors; Lung Cancer; Burkitt Lymphoma; Non-Hodgkin Lymphoma; Carcinoid Tumor; Cardiac Tumor, Medulloblastoma; CNS Embryonal Tumor, Primary CNS Lymphoma; Cervical Cancer; Cholangiocarcinoma; Chordoma; Leukemia; Lymphocytic Leukemia; Myeloid Leukemia; Myelogenous Leukemia; Myeloproliferative Neoplasm; Rectal Cancer; Craniopharyngioma; Cutaneous T-Cell Lymphoma; Mycosis Fungoides; Ductal Carcinoma In Situ; Endometrial Cancer; Uterine Cancer; Ependymoma; Esophageal Cancer; Esthesioneuroblastoma; Fallopian Tube Cancer; Gallbladder Cancer; Gastric Cancer; Gastrointestinal Carcinoid Tumor; Gastrointestinal Stromal Tumor; Gestational Trophoblastic Disease; Hepatocellular Cancer; Hodgkin Lymphoma; Hypopharyngeal Cancer; Intraocular Melanoma; Islet Cell Tumor, Pancreatic Neuroendocrine Tumor; Langerhans Cell Histiocytosis; Laryngeal Cancer; Lip and Oral Cavity Cancer; Liver Cancer; Lung Cancer, Pleuropulmonary Blastoma, Tracheobronchial Tumor; Lymphoma; Melanoma; Melanoma, Intraocular cancer; Merkel Cell Carcinoma; Mesothelioma, Metastatic Squamous Neck Cancer; Midline Tract Carcinoma With NUT Gene Changes; Mouth Cancer; Multiple Endocrine Neoplasia Syndromes; Multiple Myeloma; Myelodysplastic Syndromes, Myelodysplastic/Myeloproliferative Neoplasm; Myeloproliferative Neoplasm; Nasal Cavity cancer; Paranasal Sinus Cancer; Nasopharyngeal Cancer; Non-Hodgkin Lymphoma; Non-Small Cell Lung Cancer; Pleuropulmonary Blastoma; Oropharyngeal Cancer; Osteosarcoma; Ovarian Cancer; Pancreatic Cancer; Papillomatosis; Paraganglioma; Parathyroid Cancer; Penile Cancer; Pheochromocytoma; Pituitary Tumor; Plasma Cell Neoplasm; Breast Cancer; Lymphoma; Primary Central Nervous System Lymphoma; Peritoneal Cancer; Prostate Cancer; Recurrent Cancer; Renal Cell Cancer; Retinoblastoma; Rhabdomyosarcoma, Salivary Gland Cancer; Sarcoma; Skin Cancer; Small Intestine Cancer; Soft Tissue Sarcoma; Squamous Cell Carcinoma; T-Cell Lymphoma, Testicular Cancer; Thymoma and Thymic Carcinoma; Thyroid Cancer; Urethral Cancer; Uterine Cancer, Endometrial; Uterine Sarcoma; Vaginal Cancer; Vascular Tumors; Vulvar Cancer; Chondrosarcoma; Osteosarcoma; Rhabdomyosarcoma; Heart cancer; Astrocytoma; Brainstem glioma; Pilocytic astrocytoma; Ependymoma; Primitive neuroectodermal tumor; Cerebellar astrocytoma; Cerebral astrocytoma; Glioma; Medulloblastoma; Neuroblastoma; Oligodendroglioma; Pineal astrocytoma; Pituitary adenoma; Visual pathway and hypothalamic glioma; Invasive lobular carcinoma; Tubular carcinoma; Invasive cribriform carcinoma; Medullary carcinoma; Phyllodes tumor; Multiple endocrine neoplasia syndrome; Uveal melanoma; Appendix cancer; cholangiocarcinoma; Carcinoid tumor, gastrointestinal; Colon cancer; Extrahepatic bile duct cancer; Gastrointestinal stromal tumor; Hepatocellular cancer; Pancreatic cancer; Endometrial cancer; Renal cell carcinoma; transitional cell cancer; Gestational trophoblastic tumor; Wilms tumor; Oral cancer; Paranasal sinus and nasal cavity cancer; Pharyngeal cancer; Salivary gland cancer; Acute biphenotypic leukemia; Acute eosinophilic leukemia; Acute lymphoblastic leukemia; Acute myeloid leukemia; Acute myeloid dendritic cell leukemia; AIDS-related lymphoma; Anaplastic large cell lymphoma; Angioimmunoblastic T-cell lymphoma; B-cell prolymphocytic leukemia; Burkitt's lymphoma; Chronic lymphocytic leukemia; Chronic myelogenous leukemia; Cutaneous T-cell lymphoma; Diffuse large B-cell lymphoma; Follicular lymphoma; Hepatosplenic T-cell lymphoma; Hodgkin's lymphoma; Hairy cell leukemia; Intravascular large B-cell lymphoma; Large granular lymphocytic leukemia; Lymphoplasmacytic lymphoma; Lymphomatoid granulomatosis; Mantle cell lymphoma; Marginal zone B-cell lymphoma; Mast cell leukemia; Mediastinal large B cell lymphoma; Myelodysplastic syndromes; Mucosa-associated lymphoid tissue lymphoma; Mycosis fungoides; Nodal marginal zone B cell lymphoma; Precursor B lymphoblastic leukemia; Primary central nervous system lymphoma; Primary cutaneous follicular lymphoma; Primary cutaneous immunocytoma; Primary effusion lymphoma; Plasmablastic lymphoma; Splenic marginal zone lymphoma; T-cell prolymphocytic leukemia; Skin adnexal tumors; sebaceous carcinoma; Merkel cell carcinoma; Sarcomas of primary cutaneous origin; dermatofibrosarcoma protuberans; Bronchial adenoma and carcinoid; Mesothelioma; Pleuropulmonary blastoma; Kaposi sarcoma; Epithelioid hemangioendothelioma; Desmoplastic small round cell tumor; and Liposarcoma.

When the immune-related disorder is a lymphoproliferative disorder it may be selected from the group: post-transplant lymphoproliferative disorder; autoimmune lymphoproliferative syndrome; lymphoid interstitial pneumonia; Epstein-Barr virus-associated lymphoproliferative diseases; Waldenström's macroglobulinemia; Wiskott-Aldrich syndrome; Lymphocyte-variant hypereosinophilia; Pityriasis Lichenoides; and Castleman disease.

III Administration of the Bacteria-Mediated Delivery Vector Comprising a Therapeutic Payload

The recombinant bacterial cell of the invention for use in the prevention and/or treatment of an immune-related disorder in a subject in need thereof, is suitable for administration to the subject by a mode of administration selected from the group: intravenous, intra-arterial, intraperitoneal, intralymphatic, sub-cutaneous, intradermal, intramuscular, intraosseous infusion, intra-abdominal, oral, intratumor, intravascular, intravenous bolus; and intravenous drip.

Preferably the mode of administration is either intravenous, or intralymphatic, or intraperitoneal administration.

### EXAMPLES

General Methodology

Bacterial strains, plasmids, genes and cell lines used in the examples are identified in Table 4.

Escherichia coli strain TOP10 (Thermo Fischer Scientific) was used for DNA manipulations, plasmid propagations, and transfer experiments; while Escherichia coli Nissle 1917-pMUT1 (EcN), E. coli T7, or EcN Tn7::GFP, containing a chromosomally integrated GFP gene, were used for plasmid validations and transfer experiments. Bacterial strains were grown at 37° C. in Luria-Bertani (LB) broth or agar.

Jurkat clone E6-1 cells were maintained in Gibco™ RPMI 1640 Medium (ATCC modification, Fischer Scientific) supplemented with 10% Fetal Bovine Serum (FBS, RM10432, HiMedia Laboratories) and 1% Penicillin/Streptomycin/Neomycin (5,000 units penicillin, 5 mg streptomycin and 10 mg neomycin/ml, P4083, Sigma Aldrich) or 500× Mycozap Plus CL (Stemcell Technologies) at 37° C. and 5% CO₂. The PD-L1+ human breast cancer cell line MCF-7 was maintained in ATCC-formulated Eagle's Minimum Essential Medium (ATCC Catalog No. 30-2003) supplemented with 0.01 mg/ml human recombinant insulin, fetal bovine serum to a final concentration of 10%, and 1% Penicillin/Streptomycin/Neomycin (5,000 units penicillin, 5 mg streptomycin and 10 mg neomycin/ml, P4083, Sigma Aldrich) or 500× Mycozap Plus CL (Stemcell Technologies) at 37° C. and 5% CO₂. The CT26 murine colorectal carcinoma cell line (ATCC CRL-2638) was maintained in Dulbecco's modified Eagle's medium supplemented with 10% heat inactivated fetal bovine serum (FBS), and 1% Penicillin/Streptomycin/Neomycin (5,000 units penicillin, 5 mg streptomycin and 10 mg neomycin/ml, P4083, Sigma Aldrich) at 37° C. and 5% CO₂. The NF-κB Reporter Jurkat Cell line (Jurkat-Luc, BPS Bioscience) was maintained in Gibco™ RPMI 1640 Medium (ATCC modification, Fischer Scientific) supplemented with 10% Fetal Bovine Serum (FBS, RM10432, HiMedia Laboratories), 1 mg/ml of Geneticin, and 500× Mycozap Plus CL (Stemcell Technologies) at 37° C. and 5% CO₂

Primary human T cells and human PBMCs were maintained in ImmunoCult XF T cell Expansion medium (Stemcell technologies) supplemented with Human Recombinant IL-2 (Stemcell technologies) and 1% Penicillin/Streptomycin/Neomycin (5,000 units penicillin, 5 mg streptomycin and 10 mg neomycin/ml, P4083, Sigma Aldrich) or 500× Mycozap Plus CL (Stemcell Technologies) at 37° C. and 5% CO₂. Primary human T cells were activated with ImmunoCult™ Human CD3/CD28 T Cell Activator (Stemcell technologies).

Statistical Analysis

Quantified results were displayed as means of technical replicates±standard error of the mean (SEM), unless otherwise stated. Data sets with more than one grouping variable were assessed for statistical differences using a two-way ANOVA test with an appropriate test for multiple comparison of experimental groups, as stated in figure legends. For data sets with only one grouping variable a one-way ANOVA was used to determine significant differences. Results were considered significant with p-values below 0.05. All statistical analyses were performed using the Prism 9 software (GraphPad).

TABLE 4
Bacterial strains, plasmids, genes and cell lines of the examples

| Bacterial strain | | |
|---|---|---|
| names* | Genetic features | Source |
| E. coli TOP10 | F− mcrA Δ(mrr-hsdRMS-mcrBC) φ80lacZΔM15 ΔlacX74 recA1 | Thermo |
| | araD139 Δ(ara-leu)7697 galU galK λ− rpsL(StrR) endA1 nupG | Fischer |
| | | Scientific |
| EcN | Wild-type E. coli Nissle 1917 (EcN) | Ardeypharm |
| | | GmbH |
| EcN-MUT1 | E. coli Nissle 1917 (EcN) cured on native plasmid MUT1 | This study |
| EcN-MUT1 ΔdapA | E. coli stain EcN-MUT1 deleted for dapA gene (SEQ ID | This study |
| | No.: 247) | |
| EcN Tn7::GFP | E. coli Nissle 1917 (EcN) with chromosomally integrated GFP | this study |
| | gene (SEQ ID No.: 134) by insertion into the Tn7 gene. | |
| E. coli T7 | Chemically competent E. coli K12 derivative. F′ lac, pro, lacIq/ | New England |
| | Δ(ara-leu)7697 araD139 fhuA2 lacZ::T7 gene1 | Biolabs |
| | Δ(phoA)PvuII phoR ahpC* galE (or U) galK λatt::pNEB3-r1- | |
| | cDsbC (SpecR, lacIq) ΔtrxB rpsL150(StrR) Δgor Δ(malF)3 | |
| E. coli BM2710 | with chromosomal deletion of the dapA gene. thi-1, endA1, | Courvalin et |
| ΔdapA | hsdR17 (rk− mk+), supE44, Δ(lac)X74, ΔdapAΩcat, recA1 | al 1995 |

*Sequence for the Tn7::GFP (SEQ ID NO.: 246) can be found in the seqence list.
Sequence for dapA (SEQ ID NO.: 247) can be found in the sequence list.

| Plasmid/Vector | | |
|---|---|---|
| name ** | Genetic features | Source |
| pMUT1 | Native plasmid of E. coli Nissle 1917 | Genbank |
| (SEQ ID No.: 49) | | A84793.1 |
| pMB-dapA | Plasmid comprising nucleotide (nt) sequences of the | This study |
| (SEQ ID No.: 50) | J23119(SpeI) promoter (SEQ ID No.: 51); dapA gRNA target | |
| | (SEQ ID No.: 52); gRNA scaffold (SEQ ID No.: 53); spacer | |
| | (SEQ ID No.: 54); rrnB T1 terminator (SEQ ID No.: 55); | |
| | Bacterial terminator for E. coli rRNA rrnC (SEQ ID No.: 56); | |
| | cmR promoter (SEQ ID No.: 57) and cmR gene (SEQ ID | |
| | No.: 58); pHM 154 ori (SEQ ID No.: 60). | |
| pHM156 | Plasmid comprising nucleotide (nt) sequences of | This study |
| (SEQ ID No.: 61) | J23119(SpeI) promoter (SEQ ID No.: 51); CRISPR Cas9 gene | |
| | (SEQ ID No.: 62); FLAG (SEQ ID No.: 64); AmpR promoter | |
| | (SEQ ID No.: 65) and AmpR gene (SEQ ID No.: 66); pSC101 | |
| | ori (SEQ ID No.: 68); Rep101 (SEQ ID No.: 69); λ tL3 | |
| | terminator (SEQ ID No.: 70); rrnB T1 terminator (SEQ ID | |
| | No.: 55); spacer (SEQ ID No.: 54); gRNA scaffold (SEQ ID | |
| | No.: 53); lacI promoter (SEQ ID No.: 71); tetR gene (SEQ ID | |
| | No.: 72); arcA terminator (SEQ ID No.: 74); tonB terminator | |
| | (SEQ ID No.: 75); araC gene (SEQ ID No.: 76); araBAD | |
| | promoter (SEQ ID No.: 78); Gam gene (SEQ ID No.: 79); Beta | |
| | gene (SEQ ID No.: 81); Exo gene (SEQ ID No.: 83). | |
| pHM154 | Plasmid comprising nucleotide (nt) sequences of | This study |
| (SEQ ID No.: 85) | J23119(SpeI) promoter (SEQ ID No.: 51); gRNA spacer for | |
| | trpR (SEQ ID No.: 86); gRNA scaffold (SEQ ID No.: 53); spacer | |
| | (SEQ ID No.: 54); rrnB T1 terminator (SEQ ID No.: 55); cmR | |
| | promoter (SEQ ID No.: 57) and cmR gene (SEQ ID No.: 58); | |
| | ori (SEQ ID No.: 60). | |
| pZA11-MSC | Plasmid comprising BBa_J23100 promoter (SEQ ID | Andreas |
| (SEQ ID NO. 263) | NO.: 145); rrnB T1 terminator (SEQ ID No.: 55), p15A ori | Porse, DTU |
| | (SEQ ID No.: 95), lambda t0 terminator (SEQ ID NO.: 97), | |
| | AmpR (SEQ ID NO.: 66), AmpR promoter (SEQ ID NO.: 65) | |
| PL0017 | Plasmid comprising nucleotide (nt) sequences of CMV | Lund, A. M., |
| (SEQ ID NO. 122) | enhancer (SEQ ID No.: 123); CMV promoter (SEQ ID No.: | et al. 2014 |
| | 124); monomeric red fluorescent protein gene (mCherry) | |
| | (SEQ ID No.: 125); bGH poly(A) signal (SEQ ID No.: 127); | |
| | SV40 promoter (SEQ ID No.: 128); HygR gene (SEQ ID No.: | |
| | 129) encoding hygromycin resistance; SV40 poly(A) signal | |
| | (SEQ ID No.: 131); AmpR promoter (SEQ ID No.: 65); | |
| | AmpR gene (SEQ ID No.: 66); ori (SEQ ID No.: 60); lac | |
| | operator (SEQ ID NO.: 243); lac promoter (SEQ ID NO.: 244) | |
| pV3 | pV3 (pza11-invyp_hlyw491a) plasmid comprising nucleotide | This study |
| (SEQ ID No.: 87) | (nt) sequences of J23118 promoter (SEQ ID No.: 88); RBS | |
| | (5009.62 au) (SEQ ID No.: 89); Y. pseudotuberculosis invasin | |
| | (inv) (SEQ ID No.: 90); RBS (5966.49 au) (SEQ ID No.: 92); | |
| | L. monocytogenes listeriolysin O (hly) with a W491A | |
| | substitution (SEQ ID No.: 93); rrnB T1 terminator (SEQ ID | |
| | No.: 55); AmpR promoter (SEQ ID No.: 65) and AmpR gene | |
| | (SEQ ID No.: 66); λ tL3 terminator (SEQ ID No.: 70); p15A ori | |
| | (SEQ ID No.: 95). | |
| pSEVA27 | rrnB T1 terminator (SEQ ID No.: 55), pSC101ori (SEQ ID No.: | Silva-Rocha |
| (SEQ ID NO. 264) | 68), Rep101 (SEQ ID No.: 69), oriT (SEQ ID No.: 265), KanR | et al 2013 |
| | gene (SEQ ID No.: 104), lambda t0 terminator (SEQ ID | |
| | NO.: 97) | |
| pSQ11 | pSQ11_inv-hly plasmid comprising nucleotide (nt) sequences | This study |
| (SEQ ID NO.: 98) | of a pSC101ori (SEQ ID No.: 68); Rep101 origin (SEQ ID No.: | |
| | 69); a BamHI fragment of Y. pseudotuberculosis plasmid | |
| | pr1203 containing native inv expression cassette (SEQ ID No.: | |
| | 99) and Sau96I chromosomal fragment of Listeria | |
| | monocytogenes EGDe (NC_003210.1) containing the native | |
| | hly expression cassette (SEQ ID No.: 100); Mpl_Zinc | |
| | metalloproteinase precursor gene (SEQ ID No.: 101). rrnB T1 | |
| | terminator (SEQ ID No.: 55); λ t0 terminator (SEQ ID No.: 97); | |
| | Kan promoter (SEQ ID No.: 103); KanR gene (SEQ ID | |
| | No.: 104). | |
| pGB2Ωinv-hly | Plasmid comprising pSC101 ori (SEQ ID NO.: 68), Rep101 | This study |
| (SEQ ID NO.: 249) | (SEQ ID NO.: 69), aadA (SEQ ID NO. 250), Invasin | |
| | BZ22_RS15195 (SEQ ID NO. 252), listeriolysin precursor hlyA | |
| | (SEQ ID NO. 254), Imos06 (SEQ ID NO. 256), mpl (SEQ ID | |
| | NO. 257) | |
| pGB3 | Plasmid comprising Invasin BZ22_RS15195 (SEQ ID NO. 252), | This study |
| (SEQ ID NO.: 259) | listeriolysin precursor hlyA (SEQ ID NO. 254), Imos06 (SEQ ID | |
| | NO. 256), mpl (SEQ ID NO. 257), pSC101 ori (SEQ ID | |
| | NO.: 68), Rep101 (SEQ ID NO.: 69), lambda t0 terminator | |
| | (SEQ ID NO.: 97), AmpR (SEQ ID NO.: 66), AmpR promoter | |
| | (SEQ ID NO.: 65) | |
| pGB4 | Plasmid comprising Invasin BZ22_RS15195 (SEQ ID NO. 252), | This study |
| (SEQ ID NO.: 260) | listeriolysin precursor hlyA (SEQ ID NO. 254), Imos06 (SEQ ID | |
| | NO. 256), mpl (SEQ ID NO. 257), pSC101 ori (SEQ ID | |
| | NO.: 68), Rep101 (SEQ ID NO.: 69), lambda t0 terminator | |
| | (SEQ ID NO.: 97), AmpR (SEQ ID NO.: 66), OspF | |
| | phosphothreonine lyase (SEQ ID NO. 261), AmpR promoter | |
| | (SEQ ID NO.: 65) | |
| pCas9-gRNA-hPD1 | Plasmid comprising J23119(SpeI) promoter (SEQ ID | This study |
| (SEQ ID NO.: 197) | NO.: 51); gRNA for PDCD1 (SEQ ID NO.: 198); gRNA scaffold | |
| | (SEQ ID NO.: 53); rrnB T1 terminator (SEQ ID NO.: 55); | |
| | J23105 promoter (SEQ ID NO.: 199); 3xFLAG (SEQ ID | |
| | NO.: 200); SV40 NLS (SEQ ID NO.: 202); Cas9 (SEQ ID | |
| | NO.: 62); nucleoplasmin NLS (SEQ ID NO.: 204); ori (SEQ ID | |
| | NO.: 60); AmpR (SEQ ID NO.: 66); AmpR promoter (SEQ ID | |
| | NO.: 65). | |
| pCas9-GFP-gRNA- | Plasmid comprising U6 promoter (SEQ ID NO.: 185); gRNA | Addgene |
| hPD1 | for PDCD1 (SEQ ID NO.: 198); gRNA scaffold (SEQ ID | plasmid # |
| (SEQ ID NO.: 206) | NO.: 55); CMV enhancer (SEQ ID NO.: 123); chicken beta- | 48138 |
| | actin promoter (SEQ ID NO.: 207); hybrid intron (SEQ ID | |
| | NO.: 208); 3xFLAG (SEQ ID NO.: 200); SV40 NLS (SEQ ID | |
| | NO.: 202); Cas9 (SEQ ID NO.: 62); nucleoplasmin NLS (SEQ | |
| | ID NO.: 204); T2A (SEQ ID NO.: 209); EGFP (SEQ ID | |
| | NO.: 211); bGH poly(A) signal (SEQ ID NO.: 127); AAV2 ITR | |
| | (SEQ ID NO.: 213); f1 ori (SEQ ID NO.: 137); AmpR promoter | |
| | (SEQ ID NO.: 65); AmpR gene (SEQ ID NO.: 66); ori (SEQ ID | |
| | NO.: 60). | |
| pSpCas9(BB)-2A- | Plasmid comprising U6 promoter (SEQ ID NO.: 185); gRNA | Addgene |
| GFP | scaffold (SEQ ID NO.: 55); CMV enhancer (SEQ ID NO.: 123); | plasmid # |
| (SEQ ID NO.: 239) | chicken beta-actin promoter (SEQ ID NO.: 207); hybrid intron | 48138 |
| | (SEQ ID NO.: 208); 3xFLAG (SEQ ID NO.: 200); SV40 NLS | |
| | (SEQ ID NO.: 202); Cas9 (SEQ ID NO.: 62); nucleoplasmin | |
| | NLS (SEQ ID NO.: 204); T2A (SEQ ID NO.: 209); EGFP (SEQ | |
| | ID NO.: 211); bGH poly(A) signal (SEQ ID NO.: 127); AAV2 | |
| | ITR (SEQ ID NO.: 213); f1 ori (SEQ ID NO.: 137); AmpR | |
| | promoter (SEQ ID NO.: 65); AmpR gene (SEQ ID NO.: 66); ori | |
| | (SEQ ID NO.: 60). | |
| pNleBE | Plasmid comprising BBa_J23118 promoter (SEQ ID NO.: 88); | This study |
| (SEQ ID NO.: 214) | RBS3 (SEQ ID NO.: 215); nleB gene (SEQ ID NO.: 216); RBS4 | |
| | (SEQ ID NO.: 218); nleE gene (SEQ ID NO.: 219); rrnB T1 | |
| | terminator (SEQ ID NO.: 55); ori (SEQ ID NO.: 60); KanR (SEQ | |
| | ID NO.: 104); KanR promoter (SEQ ID NO.: 103). | |
| pUC57-ansB | Plasmid comprising BBa_J23100 promoter (SEQ ID NO.: 145); | This study |
| (SEQ ID NO.: 221) | RBS (98.3%) (SEQ ID NO.: 146); ansB gene (NCBI: | |
| | NP_415200.1) (SEQ ID NO.: 222); rrnB T1 terminator (SEQ | |
| | ID NO.: 55); ori (SEQ ID NO.: 60); AmpR (SEQ ID NO.: 66); | |
| | AmpR promoter (SEQ ID NO.: 65). | |
| pUC57-kan | Plasmid comprising lacZ-alpha (SEQ ID NO.: 241); lac | Genescript |
| (SEQ ID NO.: 240) | operator (SEQ ID NO.: 243); lac promoter (SEQ ID NO.: 244); | |
| | CAP binding site (SEQ ID NO.: 245); ori (SEQ ID NO.: 60); | |
| | KanR gene (SEQ ID NO.: 104); KanR promoter (SEQ ID | |
| | NO.: 103). | |
| psiRNA-CD3d | Plasmid comprising nucleotide (nt) sequences of T7 promoter | This study |
| (SEQ ID NO.: 193) | (SEQ ID No.: 155); shRNA CD3D (SEQ ID No.: 192); T7 | |
| | terminator (SEQ ID No.: 115); SV40 Poly(A) signal (SEQ ID | |
| | No.: 131); f1 ori (SEQ ID No.: 137); SV40 promoter (SEQ ID | |
| | No.: 128); PuroR gene (SEQ ID No.: 188); poly(A) signal (SEQ | |
| | ID No.: 190); AmpR promoter (SEQ ID No.: 65); AmpR gene | |
| | (SEQ ID No.: 66); ori (SEQ ID No.: 60); CMV enhancer(SEQ | |
| | ID No.: 123); CMV promoter (SEQ ID No.: 124); mCherry | |
| | gene (SEQ ID No.: 125). | |
| pCS6 | Plasmid comprising lambda t0 terminator (SEQ ID NO.: 97); | This study |
| (SEQ ID NO.: 234) | SmR (SEQ ID NO.: 235); SmR promoter (SEQ ID NO.: 237); | |
| | araC (SEQ ID NO.: 76); araBAD promoter (SEQ ID NO.: 78); | |
| | T7 RNA polymerase (SEQ ID NO.: 238); Rep101 (SEQ ID | |
| | NO.: 69); pSC101 ori (SEQ ID NO.: 68). | |
| pSO7 | Shigella flexneri phosphothreonine lyase OspF, Hygromycin | Lee et |
| (SEQ ID NO. 266) | resistance, Ampicillin resistance. | al 2018 |
| | Plasmid comprising nucleotide (nt) sequences of hygR (SEQ | |
| | ID NO.: 129), OspF phosphothreonine lyase (SEQ ID NO. | |
| | 261), ori (SEQ ID No.: 60), AmpR (SEQ ID NO.: 66), AmpR | |
| | promoter (SEQ ID NO.: 65). | |
| pZE3119-sfgfp | Plasmid comprising nucleotide (nt) sequences of J23119 | This study |
| (SEQ ID NO.: 116) | promoter (SEQ ID No.: 117); RBS (SEQ ID No.: 118); Super | |
| | folder GFP (SEQ ID No.: 119); rrnB T1 terminator (SEQ ID | |
| | No.: 55); Cat promoter (SEQ ID No.: 121); CmR gene (SEQ ID | |
| | No.: 58); λ t0 terminator (SEQ ID No.: 97); ori (SEQ ID No.: | |
| | 60). | |
| pCOLA-gp140 | Plasmid comprising nucleotide (nt) sequences of J23100 | This study |
| (SEQ ID NO.: 169) | promoter (SEQ ID No.: 145); RBS (SEQ ID No.: 170); Flu | |
| | signal peptide (SEQ ID No.: 171); GP120 gene (SEQ ID No.: | |
| | 172); 2xG5S (SEQ ID No.: 174); GP41 (Ectodomain) gene | |
| | (SEQ ID No.: 176); linker (Flu) (SEQ ID No.: 178); | |
| | Autochaperone (Flu) (SEQ ID No.: 180); β -barrel (Flu) (SEQ | |
| | ID No.: 182); rnnB T1 terminator (SEQ ID No.: 55); lacI | |
| | promoter (SEQ ID No.: 71); lacI gene (SEQ ID No.: 113); ColA | |
| | ori (SEQ ID No.: 114); AmpR promoter (SEQ ID No.: 65); | |
| | KanR gene (SEQ ID No.: 104). | |
| pGP140_Flu_hly | pCOLA-gp140 with insertion of Sau96I chromosomal | This study |
| plasmid | fragment of Listeria monocytogenes EGDe (NC_003210.1) | |
| | containing the native hly expression cassette (SEQ ID No.: | |
| | 100). | |
| pCOLA-ipaBC-inaK | Plasmid comprising nucleotide (nt) sequences of BBa_J23100 | This study |
| (SEQ ID NO.: 194) | promoter (SEQ ID No.: 145); inaK gene (SEQ ID No.: 195); | |
| | ipaB gene (SEQ ID No.: 14); ipaC gene (SEQ ID No.: 16); rrnB | |
| | T1 terminator (SEQ ID NO.: 55); lacI promoter (SEQ ID No.: | |
| | 71); lacI gene (SEQ ID No.: 113); ColA ori (SEQ ID No.: 114); | |
| | AmpR promoter (SEQ ID No.: 65); KanR gene (SEQ ID No.: | |
| | 104). | |
| pUC19 | Ampicillin resistance. | Thermo |
| (SEQ ID NO. 267) | lac promoter (SEQ ID NO.: 244); lac operator (SEQ ID | Fischer |
| | NO.: 243); lacZ-alpha (SEQ ID NO.: 241); AmpR gene (SEQ | Scientific |
| | ID No.: 66); AmpR promoter (SEQ ID No.: 65); ori (SEQ ID | |
| | No.: 60) | |
| pSW002-Pc- | Periplasmic expression of mTurquoise2 fluorescent protein, | Wilton et |
| TorA(sp)- | Tetracycline resistance, | al 2018 |
| mTurquoise2 | Plasmid comprising: pVS1 resolvase (SEQ ID NO.: 269), | |
| (SEQ ID No. 268) | pVS1 StaA (SEQ ID NO.: 271), pVS1 RepA (SEQ ID NO.: 272), | |
| | pVS1 oriV (SEQ ID NO.: 274), p15A ori (SEQ ID No.: 95), | |
| | mTurquoise (SEQ ID NO.: 275), tetR gene (SEQ ID No.: 72), | |
| | TcR (SEQ ID NO.: 275). | |
| pshRNA-scrambled | Scrambled shRNA(OSNEG20), Puromycin resistance, | Genecopoeia |
| (CSHCTR001-mU6 | Ampicillin resistance, mCherry fluorescent reporter gene. | |
| OSNEG20) | Plasmid comprising: SV40 Poly(A) signal (SEQ ID No.: 131); | |
| (SEQ ID NO. 279) | f1 ori (SEQ ID No.: 137); SV40 promoter (SEQ ID No.: 128); | |
| | PuroR gene (SEQ ID No.: 188); poly(A) signal (SEQ ID No.: | |
| | 190); AmpR promoter (SEQ ID No.: 65); AmpR gene (SEQ ID | |
| | No.: 66); ori (SEQ ID No.: 60); CMV enhancer(SEQ ID No.: | |
| | 123); CMV promoter (SEQ ID No.: 124); mCherry gene (SEQ | |
| | ID No.: 125); U6 promoter (SEQ ID NO.: 185), | |
| | shRNA(OSNEG20) (SEQ ID NO.: 280). | |
| pshRNA-CD3d-c | Targeting variant of shRNA against human CD3d transcript | Genecopoeia |
| (HSH02212-mU6- | variant 1 mRNA (NM_000732.4), Puromycin resistance, | |
| a-CD3D) | Ampicillin resistance, mCherry fluorescent reporter gene. | |
| (SEQ ID NO. 281) | Plasmid comprising: SV40 Poly(A) signal (SEQ ID No.: 131); | |
| | f1 ori (SEQ ID No.: 137); SV40 promoter (SEQ ID No.: 128); | |
| | PuroR gene (SEQ ID No.: 188); poly(A) signal (SEQ ID No.: | |
| | 190); AmpR promoter (SEQ ID No.: 65); AmpR gene (SEQ ID | |
| | No.: 66); ori (SEQ ID No.: 60); CMV enhancer(SEQ ID No.: | |
| | 123); CMV promoter (SEQ ID No.: 124); mCherry gene (SEQ | |
| | ID No.: 125); U6 promoter (SEQ ID NO.: 185 shRNA CD3D | |
| | (SEQ ID No.: 192). | |

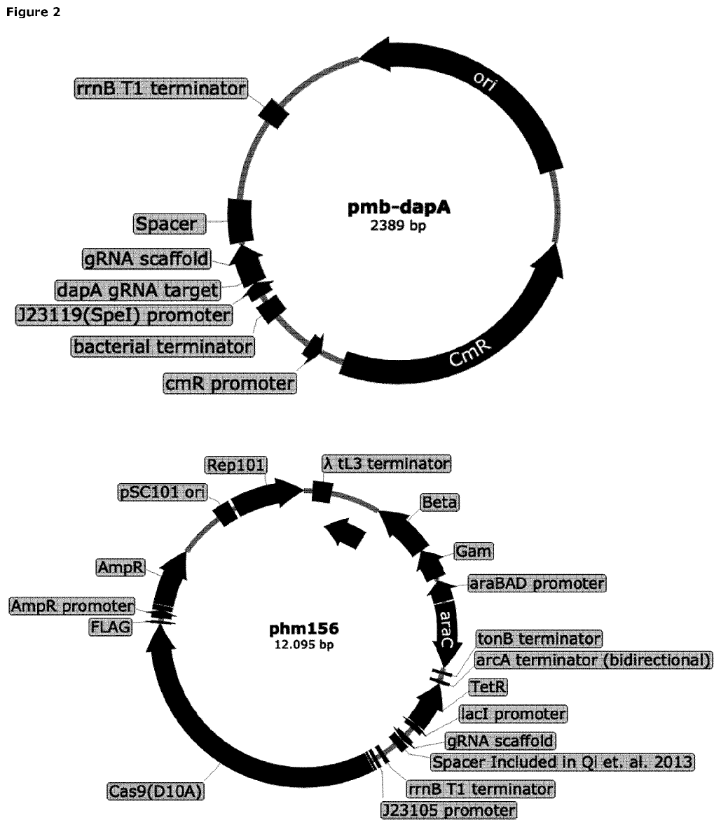

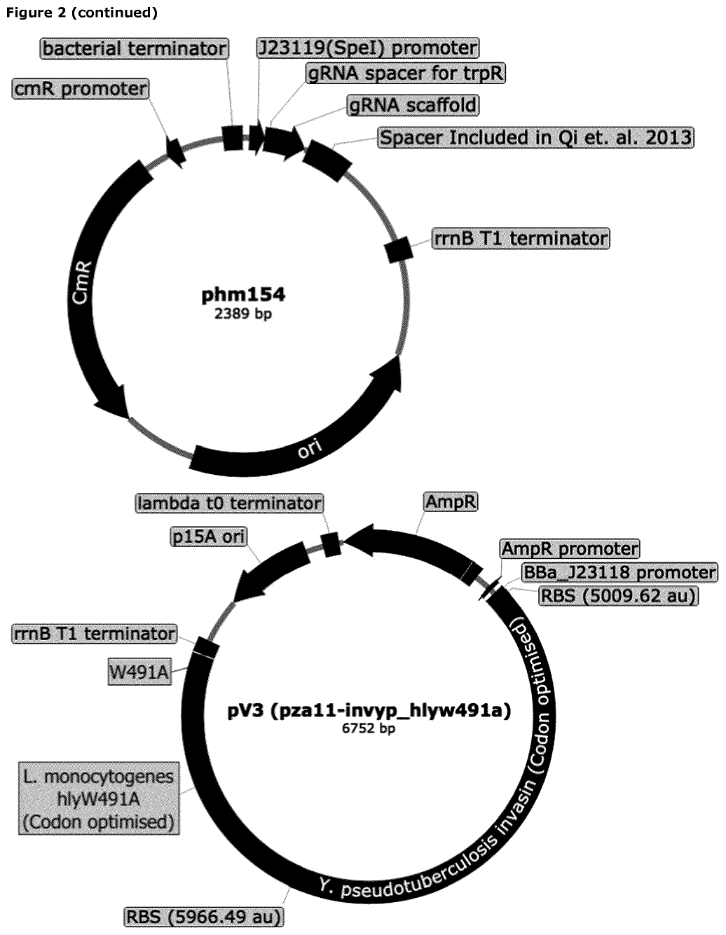

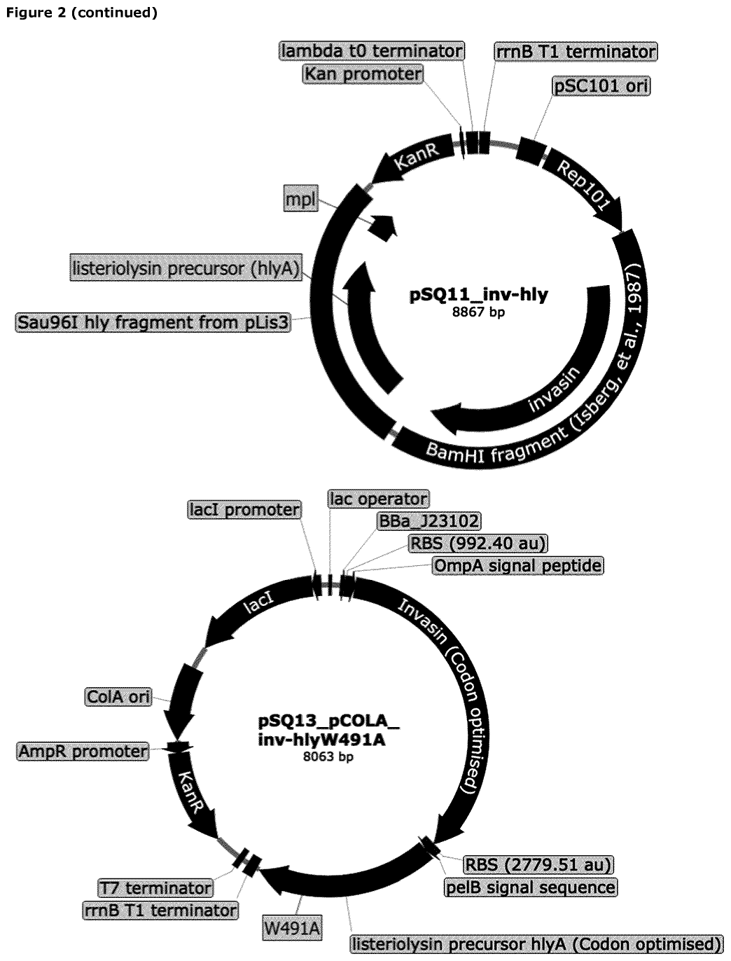

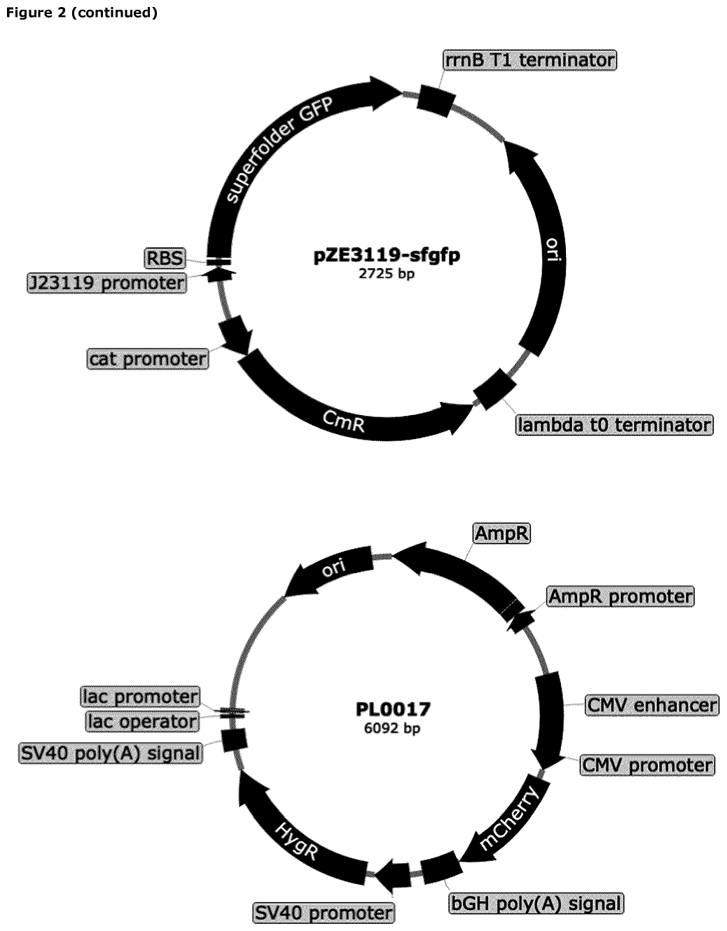

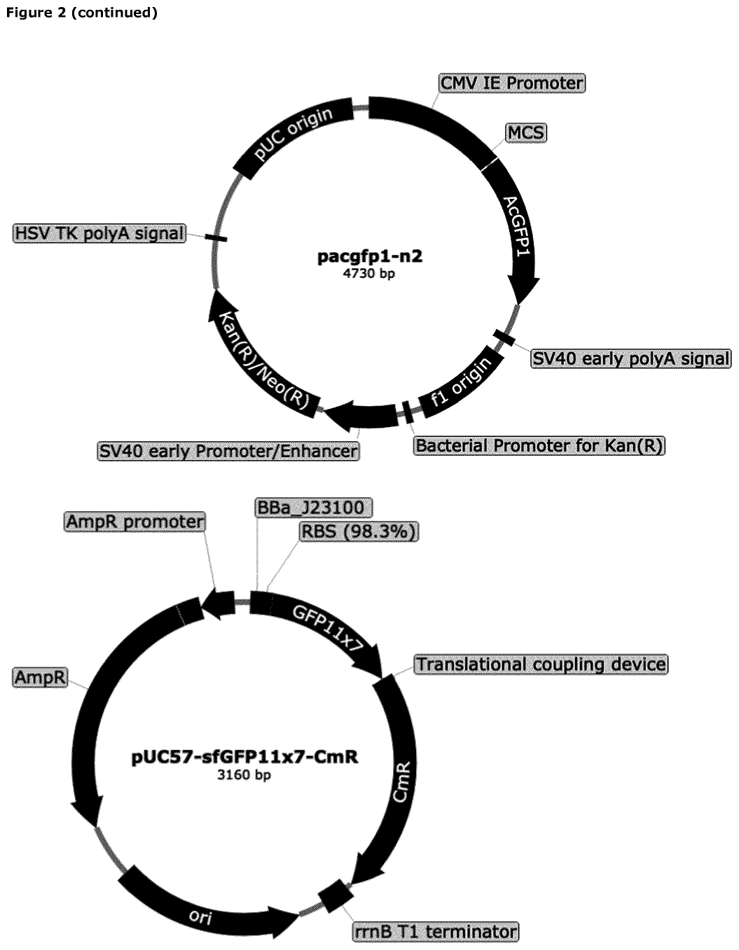

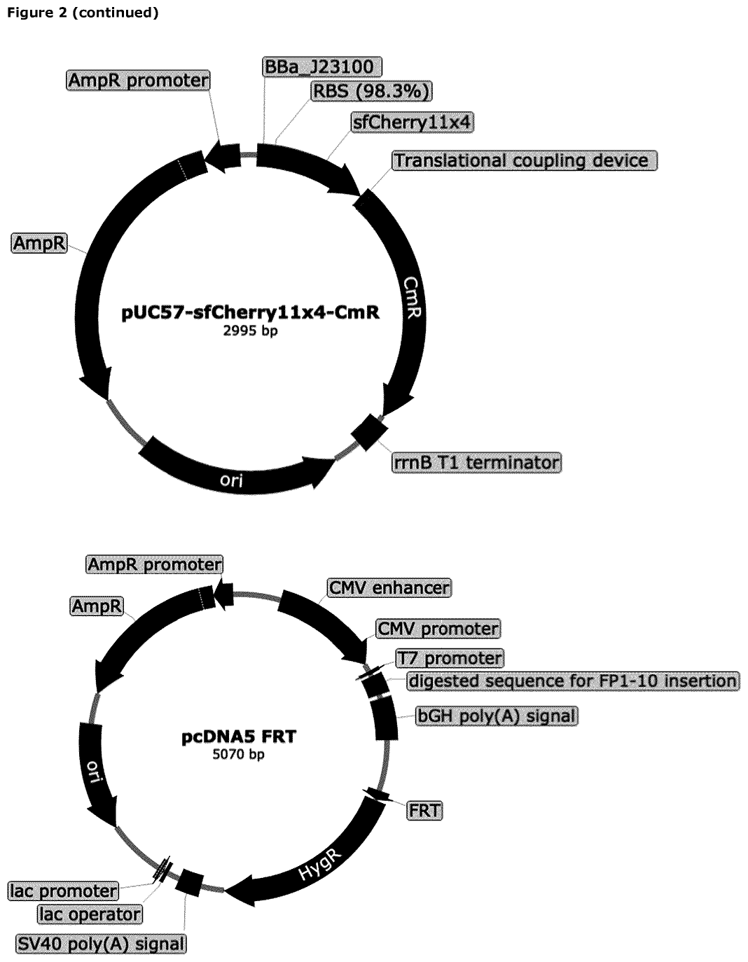

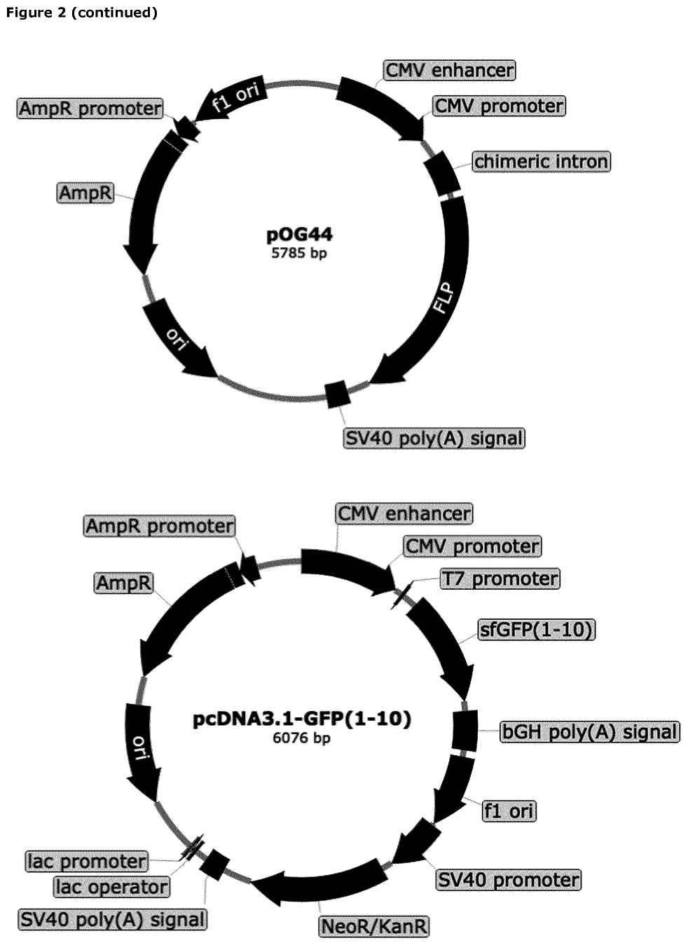

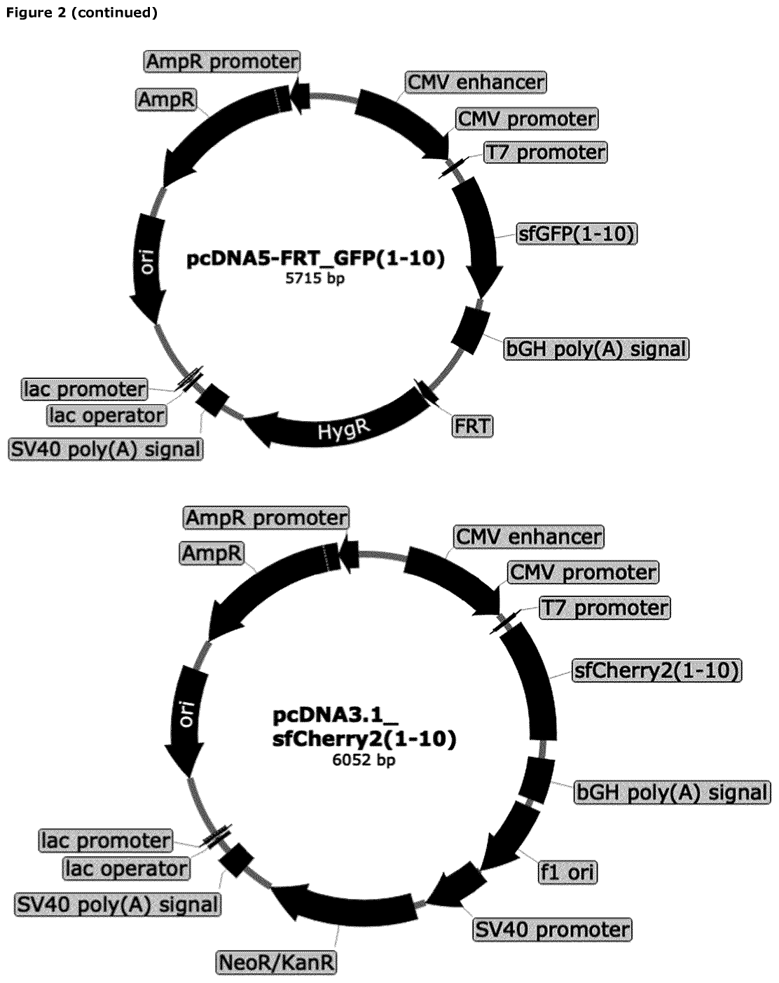

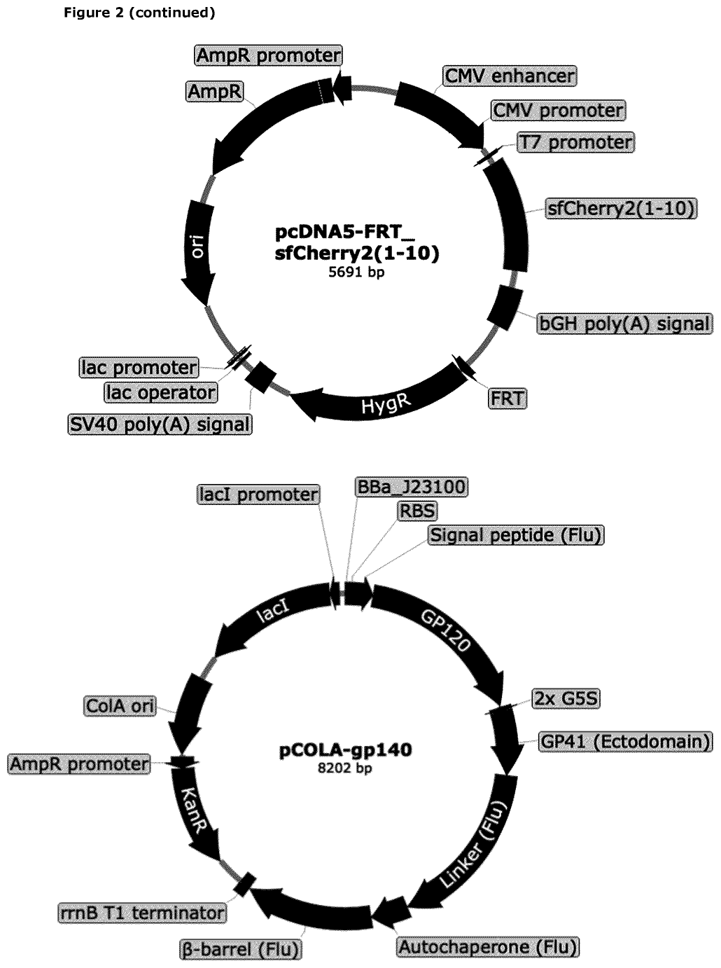

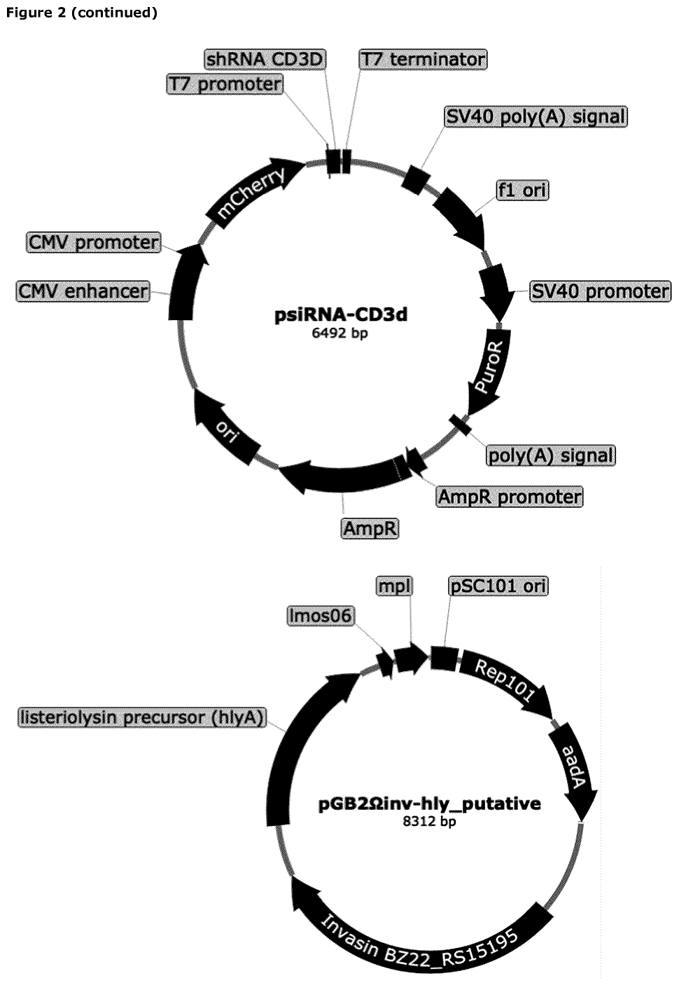

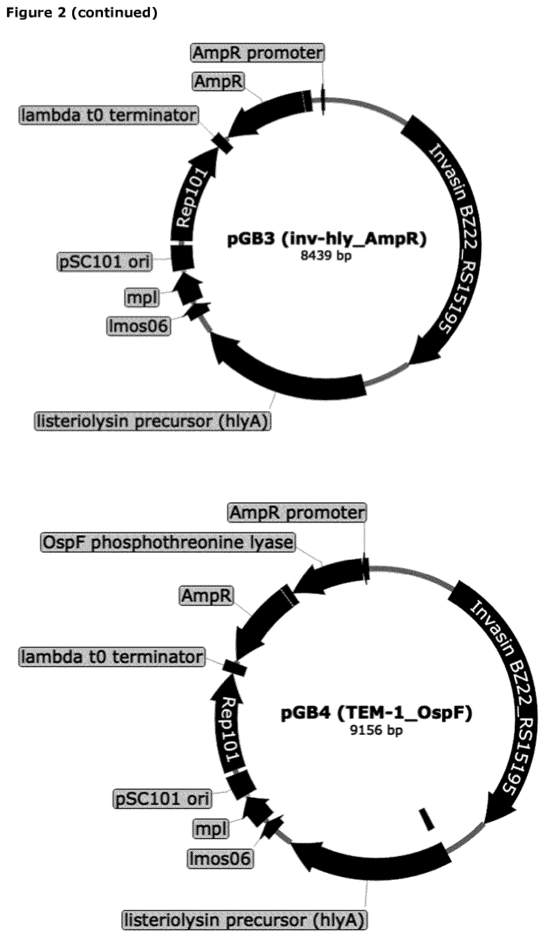

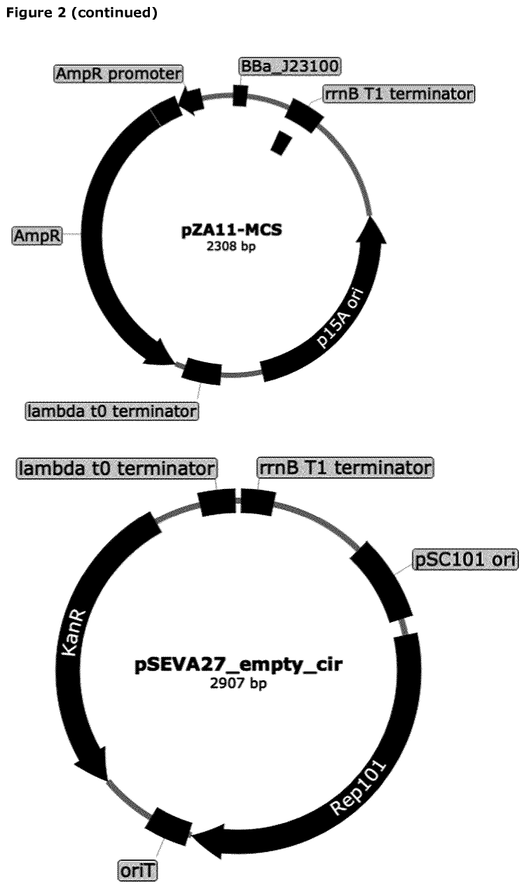

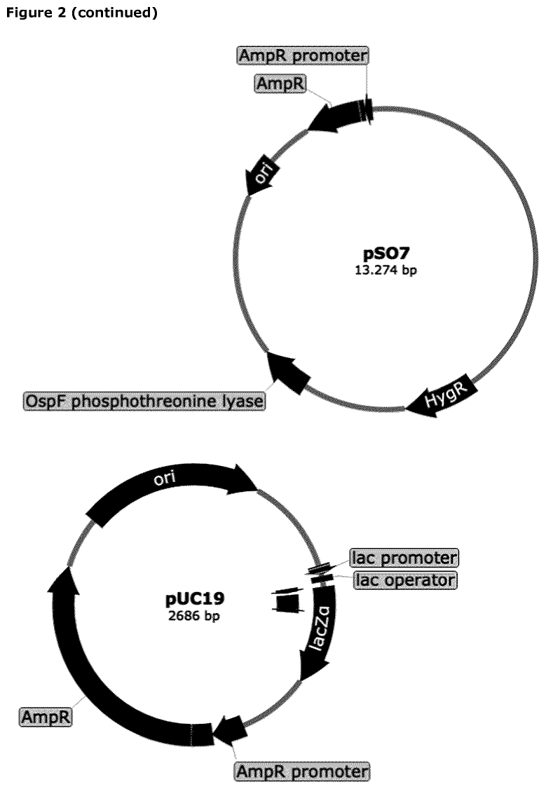

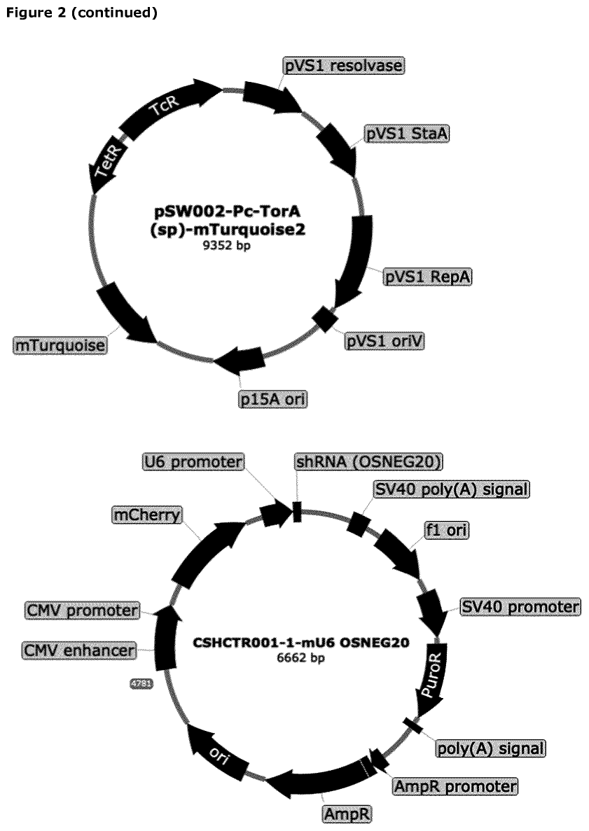

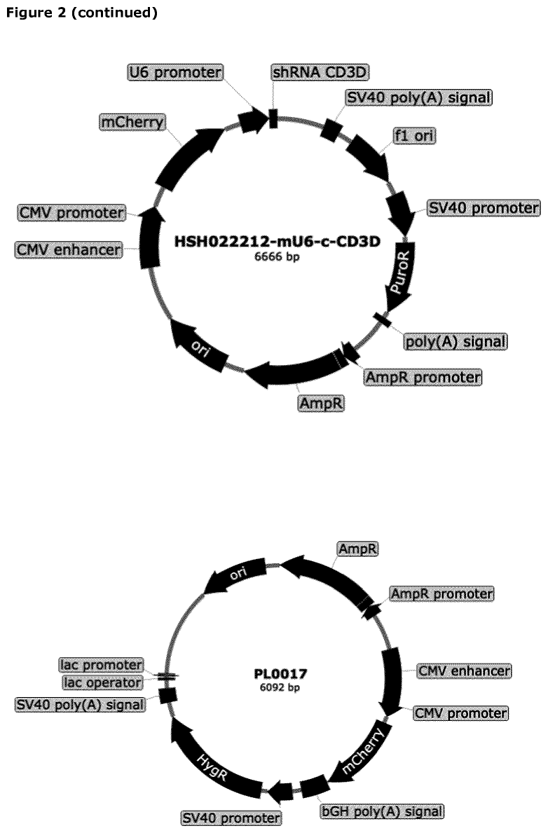

** Plasmid maps are illustrated in FIG. 2

| Cell lines | Genetic features | Source |
|---|---|---|
| Jurkat clone E6-1 | Human actute T cell leukemia cell line that | ATCC TIB-152 |
| | overproduces IL-2 after stimulation. | |
| PBMC | Human peripheral blood mononuclear cells | Donor blood |
| CT26 | CT26 murine colorectal carcinoma cells | ATCC CRL-2638 |
| PD-L1⁺ MCF-7 | PD-L1⁺ human breast cancer | (Y. Zheng, Fang, |
| | cell line MCF-7 | & Li, 2019) |
| NF-κB Reporter | NF-κB Reporter T cell line | BPS Biosciences |
| (Luc) - Jurkat Cell | | (Catalog #: 60651) |
| line (Jurkat-Luc, | | |
| BPS Bioscience) | | |

### Example 1 Engineering Bacteria-Mediated Delivery Vectors Expressing Invasion and Listeriolysin O

The parent bacterial strain E. coli Nissle 1917 (EcN), cured of one of its two native plasmids to create strain (EcN-pMUT1), was used for engineering gene or protein delivery vectors as described below (method). The native pMUT1 plasmid was cured using CRISPR for future reintroduction of an engineered plasmid containing the invasive phenotype (Zainuddin, Bai, & Mansell, 2019). Expression of the invasive phenotype on a native plasmid allows for plasmid maintenance without the need for antibiotic resistance genes. A deletion strain, EcN-pMUT1 ΔdapA, characterized by auxotrophy for diaminopimelic acid (DAP) due to deletion of chromosomal copies of the dapA gene encoding 4-Hydroxy-tetrahydrodipicolinate synthase, was derived from EcN-pMUT1, into which one of the alternative inv-hly expression plasmids (Table 4) were introduced. The inv-hly genes encode Yersinia pseudo tuberculosis invasin and Listeria monocytogenes listeriolysin O proteins that together provide two component system for delivery of genes or proteins into a mammalian non-phagocytic immune cell. The resulting inv-hly strains were further transformed with various reporter plasmids for monitoring transfer of a gene payload.

Methods:

Deletion of dapA gene: The dapA gene in EcN-pMUT1 was deleted by a modified CRISPR-Cas9 λ-Red recombinase genome editing strategy (Reisch and Prather, (2015)) using the plasmids: pMB-dapA containing the dapA guide RNA; pHM156 containing CRISPR Cas9 for cutting dsDNA and λ Red homologous recombination system (Gam, Beta and Exo genes), and pHM154 a template gRNA plasmid containing a guide RNA spacer for trpR. pMB-dapA, the gRNA plasmid, was generated by overlap PCR amplification with primers amplifying pHM154, whilst excluding the gRNA spacer region, using overlaps matching the 3′ end of dapA (2841684 nt-2842562 nt in EcN chromosome). The resulting fragment was assembled into a plasmid via standard Gibson assembly to create pMB-dapA. A homologous DNA fragment, OE-dapA was created for replacement of dapA. First, primers amplified 500 bp upstream and 500 bp downstream of the dapA gene. The two resulting fragments were then combined via PCR using the forward and reverse primers of the upstream and downstream fragment, respectively.

The host cell, EcN, was then transformed with the CRISPR Cas9 and λ Red system plasmid pHM156. The λ Red recombinase was induced by incubation with L-arabinose at 30° C. and the cells were electroporated with the gRNA plasmid pMB-dapA and the homologous OE-dapA flanking region DNA fragment. The CRISPR Cas9 complex introduced a double stranded cut in the dapA gene via guidance of the gRNA. The chromosomal DNA cut was fatal to the bacterial cells unless the cut was repaired via homologous recombination with the supplied DNA fragment and subsequent replacement of the dapA gene, resulting in the creation of strain EcNΔdapA. Successful knockouts were confirmed by PCR amplification and plasmids pMB-dapA and pHM156 were cured via incubation with anhydrotetracycline hydrochloride (Sigma Aldrich) at 37° C.

Cloning “Invasion-Listeriolysin O” Expression Plasmids:
pV3 plasmid: a DNA fragment was synthesized comprising codon-optimized genes, inv and hly, encoding a Y. pseudotuberculosis invasin and a L. monocytogenes listeriolysin O harboring a W491A mutation respectively; wherein the genes were operably linked to constitutive promoter BBa_J23118 having a measured promoter strength of 0.56 (Anderson. C., 2006) and an optimized Ribosomal Binding Site (RBS) with a predicted strength of 5009 au and 5966 au for inv and hly, respectively (Salis H M., 2011; http://emopec.biosustain.dtu.dk/). This DNA fragment was cloned into plasmid pZA11-MCS to create plasmid pV3 (Table 4), by using Gibson assembly to combine it with a PCR amplified copy of the plasmid backbone using primers with homology arms to the DNA fragment.
pSQ11 plasmid: a DNA fragment comprising a native inv gene, encoding a Y. pseudotuberculosis invasin operably linked to a eukaryotic CMV promoter, was amplified using primers having a 40 bp homology arm to a pSEVA27 plasmid. A second DNA fragment comprising a native hly gene encoding listeriolysin O was amplified from the Listeria monocytogenes EGDe chromosome. The DNA fragment comprising the hly gene was assembled downstream of the inv gene and combined with the amplified backbone of plasmid pSEVA27 using Gibson assembly to create pSQ11 (Table 4).
pGB3: The TEM-1 gene of pUC19 was cloned into pGB2Ωinv-hly to replace the aadA gene
pGB4: The OspF gene of pS07 was cloned downstream of the TEM-1 promoter as a fusion to TEM-1 in pGB3.

Cloning Reporter Plasmids:
pZE3119-sfgfp plasmid: comprises a gene encoding a super folding GFP (sfGFP) under bacterial promoter cloned into a pZE plasmid backbone.
pPL0017 plasmid: comprises a gene encoding monomeric red fluorescent protein (mCherry) driven by PcMv promoter and gene encoding hygromycin resistance driven by SV40 promoter cloned into a pBASE vector (Lund et al., 2014).

Cloning Protein Transfer Reporter Plasmids and Vectors:

The only protein transfer reporter that was made was the pGB3 which is also an invasive plasmid and hence described above.

Transformation of E. coli strains: The respective “invasion-listeriolysin O” (inv-hly) expression plasmids were individually transformed into E. coli strain EcN-pMUT1 ΔdapA, E. coli TOP10, E. coli Nissle-pMUT1, E. coli Tn7::GFP, E. coli Nissle-pMUT1, or E. coli BM2710 ΔdapA together with a reporter plasmid (e.g. GFP reporter plasmid (pZE3119-sfgfp) or the mCherry reporter plasmid (pshRNA-CD3d-c (HSH02212-mU6-c-CD3D)) by electroporation to create the strains listed in Table 4.

Cell line culture conditions: Jurkat cells were maintained in Gibco™ RPMI 1640 Medium (ATCC modification, Fischer Scientific) supplemented with 10% Fetal Bovine Serum (FBS, RM10432, HiMedia Laboratories) and 1% Penicillin/Streptomycin/Neomycin (5000 units penicillin, 5 mg streptomycin and 10 mg neomycin/ml, P4083, Sigma Aldrich) or 500× MycoZap Plus-CL (Lonza Bioscience) at 37° C. and 5% CO₂. Primary human T cells and human PBMCs were maintained in ImmunoCult XF T cell Expansion medium (Stemcell technologies) supplemented with Human Recombinant IL-2 (Stemcell technologies) and 1% Penicillin/Streptomycin/Neomycin (5,000 units penicillin, 5 mg streptomycin and 10 mg neomycin/ml, P4083, Sigma Aldrich) or 500× Mycozap Plus CL (Stemcell Technologies) at 37° C. and 5% CO₂. Primary human T cells were activated with ImmunoCult™ Human CD3/CD28 T Cell Activator (Stemcell technologies).

### Example 2 Engineering Bacteria-Mediated Delivery Vectors Expressing ipaB and ipaC

E. coli strains (EcN-MUT1 ΔdapA) engineered to express ipaBC-inaK fusion proteins are capable of functioning as bacteria-mediated delivery vectors of genetic material to a target non-phagocytic immune cell.

Methods:

Cloning “ipaBC-inaK” Expression Plasmid:
pCOLA-ipaBC-inaK plasmid: contains a gene encoding fusion proteins comprising an C-terminally truncated inaK (derivable from Pseudomona syringae) fused at its C-terminus to ipaB fused to ipaC (derivable from Shigella sonnei). The truncated INP protein comprise a GPI-anchoring domain and repeat region that allows both membrane anchoring and sufficient flexibility for ipaB ipaC interactions during complex formation. The construct was introduced into an expression plasmid together with a synthetic promoter, RBS, and termination signal. This pCOLA-ipaBC-inaK plasmid was transformed into strain EcNΔdapA.

Primary human pan T cells were diluted to 2.67×10⁵ cells/ml in pre-warmed ImmunoCult XF T Cell Expansion medium supplemented with 25 ng/ml of IL-2 and added to a PLO coated 96 well plate at 150 μl per well. Overnight cultures of EcN, carrying either invasive plasmid pCOLA_ipaBC_inaK and the sfGFP reporter plasmid pZE3119 or no plasmid as a negative control, were labelled with the Incucyte® pHrodo® Red Cell Labelling Kit for Phagocytosis (Essen Biosciences), following the manufacturer's instructions. pHrodo Red labelled bacteria were diluted to an MOI of 2000 or 5.33×10⁸ cfu/ml in complete pre-warmed cell culture media and 150 μl were added to the T cell wells. As a positive control, 30 μl of labelled WT EcN were added, without cells, to separate wells containing 270 μl citrate-based buffer solution (pH<4.0). The plate was centrifuged for 10 minutes at 100×g to initiate contact between bacteria and placed into an IncucyteS3 instrument at 37° C. and 5% CO₂. Using the cell-by-cell imaging software module, wells were imaged on all channels at 20× magnification every 20 minutes for a total of 2 hours. The Incucyte analysis software was used to create single cell masks to identify T cells and total mean red, as well as green, fluorescence intensity object averages were determined for each image.

Results

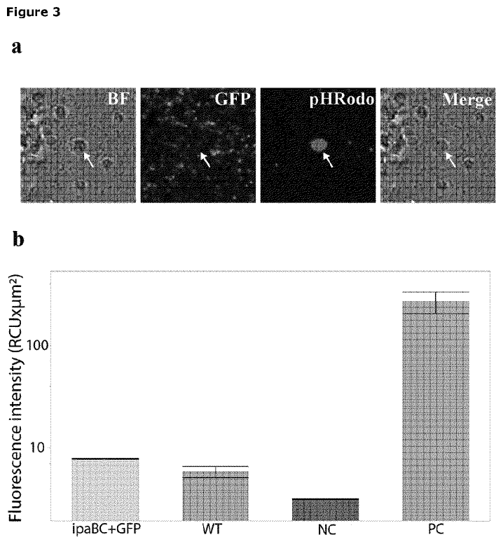

As shown in FIG. 3a, intracellular bacteria could be detected inside infected cells, as indicated by bright red fluorescence entirely filling up cells without any GFP signal. In addition to increased red fluorescence, a lack of bacterial GFP signal from infected cells was indicative of phagolysosome localization of bacteria, due to lysis and denaturation of intracellular bacterial GFP signal in acidic pH.

On average, red fluorescence was highest for cells infected with invasive bacteria after 1 h and 50 min, compared to WT or uninfected cells. Whilst WT bacteria infected cells had a significantly higher total integrated red fluorescence per cell than uninfected cells, which indicated spontaneous uptake of the bacteria, invasive bacteria infected cells were significantly brighter than WT infected ones (FIG. 3b). Positive control bacteria, resuspended in acidic citrate buffer, exhibited red fluorescence values more than 10× higher than what was observed for infected cells. It was likely that this stark difference in fluorescence signal was due to the difference in pH conditions. Whilst citrate buffer had a pH of <4, the pH of the phagolysosome depends on the stage of maturation. From the initial formation of the phagosome, when pH values can range from 6.1-6.5, several fusion and fission events have to occur before the final phagolysosme has matured and contains pH conditions of below 5.5 (Uribe-Quero & Rosales, 2017). Hence, depending on the stage of maturation that the observed infected cells were in, red fluorescence intensities from the pH or dye could be drastically lower than the positive bacterial control in citrate buffer. Also, the ipaBC complex has been shown to be involved in phagosome escape in the native Shigella host (Croxen et al., 2013). In particular, ipaB has been described to form ion pores that lyse the phagosome (Yang, Hung, Aljuffali, & Fang, 2015). Hence, the large difference in red fluorescence values were likely due to bacterial escape from the phagosome during early stages of maturation. Whilst the WT bacteria were not capable of phagosome lysis, the low fluorescence values observed with these bacteria were contributed to the significantly lower rates of internalisation. In other words, cell populations infected with invasive bacteria might have had frequent events of cells with slightly increased red fluorescence while WT infected populations might have had very few events of cells with strongly increased red fluorescence. Because of this, both conditions might have resulted in similar levels of mean red fluorescence compared to positive control bacteria.

### Example 3 Recombinant Bacteria-Mediated Delivery Vectors Expressing Invasin and Listeriolysin O Invade T Cell Line and PBMCs

Engineered E. coli strains (EcN-MUT1 ΔdapA) containing one of the inv-hly expression plasmids (pV3 or pSQ11 (Example 1) and or a combination of the inv-hly plasmids pV3 or pSQ11 together with the reporter plasmid pZE3119-sfgfp (Example 1) were tested for their ability to infect Jurkat E6-1 cells and PBMCs, and thereby shown to function as bacteria-mediated delivery vectors of genetic material to a target non-phagocytic immune cell.

Methods:

Fluorescence microscopy analysis of invasion: Infection was performed in a flat-bottom 6-well plate, using 2.5×10⁵-1.2×10⁶ Jurkat E6-1 cells or PBMCs per well. The respective cells were infected with overnight cultures of E. coli EcN-pMUT1 strains comprising an inv-hly expression plasmid together with the reporter plasmid pZE3119-sfGFP or a control EcN-pMUT1 strain with only the reporter plasmid at multiplicity of infection (MOI) of either 400 or 2000 in RPMI media (see general methods) supplemented with 10% fetal bovine serum (FBS). Plates were centrifuged at 100×g for 10 min in a swinging bucket centrifuge, to initiate contact between cells of the E. coli strains and human cells, and incubated for 1 hour at 37° C. and 5% CO₂. Next, well contents were transferred to individual 15 ml falcon tubes and washed once with Phosphate Buffered Saline (Gibco™ PBS, pH 7.4 at room temperature, Fischer Scientific) at 300×g for 5 min at RT. A sterilized microscope glass cover slip was placed in the wells of a new 6-well plate and the washed cells, re-suspended in PBS, were slowly added onto the cover slip. The plates were incubated at RT for 30 minutes to allow for adherence of cells to the cover slip via gravity sedimentation (Chowdhury, S. et al, 2017). Upon adherence, PBS was carefully aspirated from the wells, and the wells were gently washed three times with PBS.

The cells, adhered to cover slips, were then fixed for 15 minutes at RT with Image-iT Fixative Solution (Thermo Fischer Scientific). After subsequently washing the cover slips in PBS, cellular antigens on the slides were blocked for 30 min at 37° C. at 5% CO₂ with stain buffer containing 2% fetal bovine serum (FBS) and 0.09% sodium azide (BD Biosciences).
Antigen detection: the fixed cover slips were incubated with primary antibodies diluted in stain buffer for 1 hr at RT or overnight at 4° C.; using primary antibodies to E. coli LPS (Thermo PA1-25636, Thermo Fischer Scientific) and CD49D integrins on human cells (Thermo 14-0499-82, Thermo Fischer Scientific). The primary antibody staining solution was then removed and the cover slips washed three times with PBS for 5 minutes. The cover slips were then incubated with secondary antibodies diluted in stain buffer for 1 hr at RT or overnight at 4° C.; using the secondary antibodies: ATTO 550 binding to anti-E. coli LPS antibody (Sigma 43328, Sigma Aldrich); Alexa Fluor 350 binding to anti-E. coli LPS antibody (A-11046, Thermo Fischer Scientific), and Brilliant Violet 480, binding to CD49D antibody (BD 746384, BD Biosciences). The secondary antibody staining solution was then removed and the cover slips washed three times with PBS for 5 minutes. For visualization of internalized E. coli cells, the infected immune cells were permeabilized with 0.5% Triton X-100 prior to incubation with the primary LPS antibody and the secondary anti-E. coli LPS antibody Alexa Fluor 350.
Nuclear staining: the fixed covers slips were incubated with DAPI Solution (1 mg 4′,6-diamidino-2-phenylindole/mL, 62248, Thermo Fischer Scientific) for 5 min at RT in the dark, followed by a PBS wash for 5 min.
Actin staining: the fixed covers slips were incubated with 0.5% Triton X-100 in distilled water for 15 min at RT to permeabilize infected immune cells; washed twice with PBS. Two drops of rhodamine phalloidin (ActinRed™ 555 ReadyProbes from Thermo Fischer Scientific) were then added to the cover slips and incubated for 30 minutes at RT in the dark and finally washed twice with PBS.
Mounting: 25 μl of Vectashield hardmount solution (H-1400, Vector Laboratories) were added onto a microscope slide. The cover slips were carefully placed onto the microscope slide, cell side facing the mounting solution, and left to cure for 15 minutes at RT. Prepared microscope slides were imaged on a fluorescence microscope using a 60× magnification.

Incucyte Live Cell Imaging Analysis of Invasion

Jurkat E6-1 cells were diluted to 3×10⁵ cells/ml in pre-warmed RPMI+10% FCS and added to a 96 well plate at 100 μl per well. An overnight cultures of EcN Tn7::GFP containing either the invasive plasmid were diluted in complete cell culture media to MOIs ranging from 80-1280 or 2.4×10⁷-3.8×10⁸ cfu/ml. 100 μl of diluted bacteria were added to Jurkat cells and plates were centrifuged at 100×g for 30 sec to initiate contact between bacteria. Plates were incubated for 2 hours at 37° C. and 5% CO₂. To terminate infections, cell suspensions were transferred to a 96-well V-bottom plate and centrifuged for 5 minutes at 200×g. Pellets were gently washed once with 100 μl complete cell culture medium. Cells were resuspended in 200 μl of RPMI+10% FCS+50 μg/ml Gentamicin and transferred to a new PLO coated 96-well F-bottom plates and left stationary to settle at RT for 20 minutes. The plate was then transferred to an IncucyteS3 instrument at 37° C. and 5% CO₂. Using the cell-by-cell imaging software module, wells were imaged on all channels at 20× magnification every 20 minutes for a total of 4 days. The Incucyte analysis software was used to create single cell masks to identify T cells and total mean red, as well as green, fluorescence intensity object averages were determined for each image.

Results:

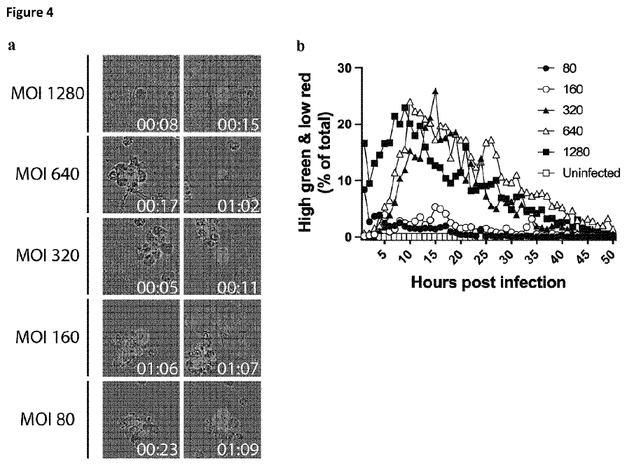

Jurkat E6-1 cells were infected with GFP expressing EcN-Tn7::GFP containing the invasive plasmid pV3, encoding codon-optimsed versions of the native inv-hly genes as well as containing a cytotoxicity reducing mutation in hly. Infected cells were then visualized in the Incucyte to determine green and red fluorescence. Cells exhibiting both green and red fluorescence were excluded from analysis as autofluorescent dying cells. Only cells that exclusively exhibited green fluorescence were determined to contain intracellular bacteria where green fluorescence stemmed from bacterially produced GFP and not cellular autofluorescence. As shown in FIG. 4a, several cells could be observed to contain increasing amounts of GFP expressing bacteria over time. At an MOI of 160, it was possible to observe one cell that burst and release a large amount of intracellular bacteria, indicative of active intracellular bacterial replication. Quantification of invaded cells in FIG. 4b showed that a maximum of around 25% of cells were invaded with bacteria and that this maximum was reached in a concentration dependent manner. Whilst the highest MOI of 1280 reached the maximum after around 10 hours post infection, an MOI of 320 only reached the maximum after around 16 hours. MOIs below 320 only achieved low invasion rates below 5%.

The invasive properties of the engineered E. coli EcN strains expressing the two component delivery system encoded by the Yersinia pseudotuberculosis invasin gene and the Listeria monocytogenes listeriolysin O system (inv-hly) and the reporter gene GFP, in T-cells (Jurkat E6-1) and human PBMCs infected with these strains was detected by immunofluorescence microscopy. All E. coli cells of the engineered EcN strains were identifiable and localizable by virtue of their expression of GFP and its detectable fluorescence. Those E. coli cells that remain external to the infected T-cells of PBMB cells (target cells) were detected by the combination of the primary E. coli LPS antibody and secondary anti-E. coli LPS antibody (ATTO 550) that detect E. coli surface antigens, since the antibodies are too large to enter the target cells and therefore intracellular bacteria. Subsequent detergent permeabilization of the target cells allowed detection of E. coli cells internalized within the target cells, using a combination of the primary E. coli LPS antibody and secondary anti-E. coli LPS antibody (ATTO 350). The target cells were detected and localized using anti-CD49D antibody that binds to integrin α4β1 on the mammalian cell surface. DNA staining with DAPI allows detection of the nucleus in both E. coli and target cells and their respective localization.

The target T-cells and PBMCs were infected with E. coli strains expressing two variants of the inv-hly two component delivery system encoded by genes on the plasmids pV3 and pSQ11 (Table 4). The invasin and Listeriolysin O encoded by the plasmids differed in respect to codon optimization of the expressed genes; the promoter strength and the substitution of E. coli signal peptides for the respective native signal peptide.

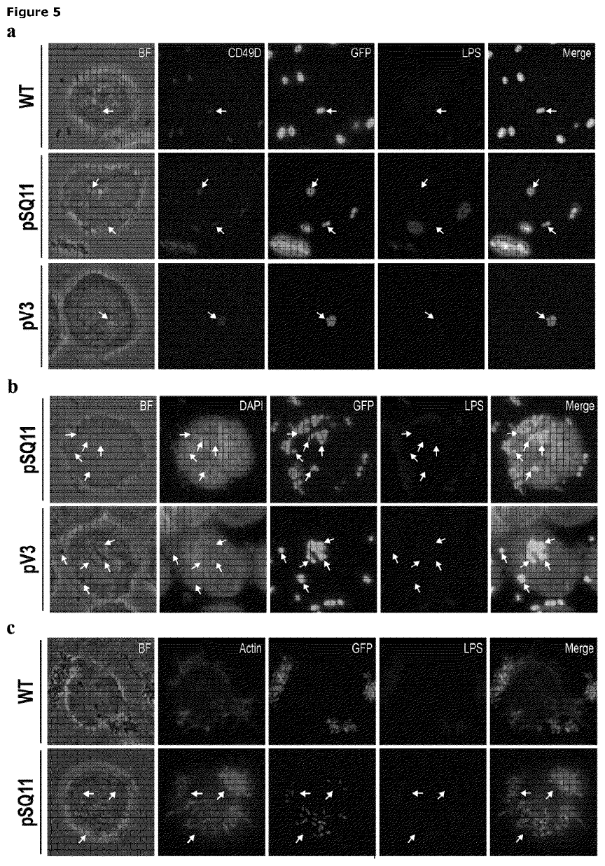

Infection of T-cells (Jurkat E6-1) (FIG. 5a&c) and PBMCs (FIG. 5b) with the engineered EcN strains expressing the two component delivery system (inv-hly) encoded by each of the three plasmids resulted in successful invasion, since several internalized E. coli cells were detected of these target cells. In contrast only a single invasion event was observed with the WT EcN strain in T-cells (see FIG. 5a, top row). The invaded PBMCs detected in FIG. 5b were presumed to be lymphocytes in view of the morphology of their DAPI stained nucleus; and the relative abundance of lymphocytes (T cells, B cells, NK cells) amongst human PBMCs, that is typically 70-90%. In some cases, intracellular bacteria were observed to be contained in phagolysosome-like compartments whilst others were not, indicating that expression of the Listeriolysin O by the invading E. coli cells successfully mediates phagolysosome escape.

When actin was detected in T-cells (Jurkat E6-1), infected with E. coli cells of the engineered EcN strain expressing the two component delivery system (inv-hly), actin polymerization was observed along points of contact with the internalized bacteria (FIG. 5c); indicating that the observed invasion may be mediated by an actin-dependent mechanism. This property was not seen for T-cells infected with the WT E. coli strain.

### Example 4 Use of Recombinant Bacteria-Mediated Delivery Vectors Expressing Invasin and Listeriolysin O to Transfer Genes into a T Cell Line

E. coli BM2710, containing a combination of the inv-hly expression plasmid pGB2Ωinv-hly and the mCherry reporter plasmid pshRNA-CD3d-c, was used to infect Jurkat E6-1 cells. Strong and persistent plasmid expression was shown in several infected cells and thereby the data demonstrated the use of bacteria-mediated delivery vectors of the invention for transfer and expression of genetic material to a target non-phagocytic immune cell.

Methods:

Jurkat E6-1 were diluted to 1×10⁵ cells/ml in 6.5 ml of PBS and stained with 65 μl of the cytoplasmic labelling dye Incucyte® Cytolight Rapid Green (Essen Bioscience) for 20 minutes at 37° C., according to the manufacturer's instructions. Excess dye was diluted by addition of 40 ml RPMI+10% FCS and cell suspensions were centrifuged at 300×g for 7 minutes. Cell pellets were resuspended in complete culture medium supplemented with diaminopimelic acid (DAP) at a final concentration of 100 μg/ml. 500 μl of labelled cells were added to a 12 well plate at a density of 4×10⁵ cells/ml. An overnight culture of E. coli BM2710 containing the invasive plasmid pGB2Ωinv-hly and the reporter plasmid pshRNA-CD3d-c was diluted to an MOI of 640 or 2.6×10⁸ cfu/ml in complete culture medium supplemented with 10 mg/ml 2,6-Diaminopimelic acid (Sigma Aldrich) and 500 μl of bacterial suspension were added to the wells. Bacteria containing only the invasive plasmid but not the reporter plasmid served as a negative control. Co-cultures were incubated for 2 hours at 37° C. and 5% CO₂. To terminate infections, well contents were pelleted, resuspended in complete cell culture medium supplemented with 50 ug/ml Gentamicin, and transferred to new PLO coated 12 well plates. The plates were transferred to an IncucyteS3 instrument and incubated at 37° C. and 5% CO₂. Using the cell-by-cell imaging software module, wells were imaged on all channels at 20× magnification every 2 hours for a total of 70 hours. The Incucyte analysis software was used to create single cell masks to identify T cells and total mean red, as well as green, fluorescence intensity object averages were determined for each image. Images that were taken after plate disturbances, due to e.g.: movement of the instrument sample tray, or that contained misidentified cells were excluded from quantitative analysis.

Results:

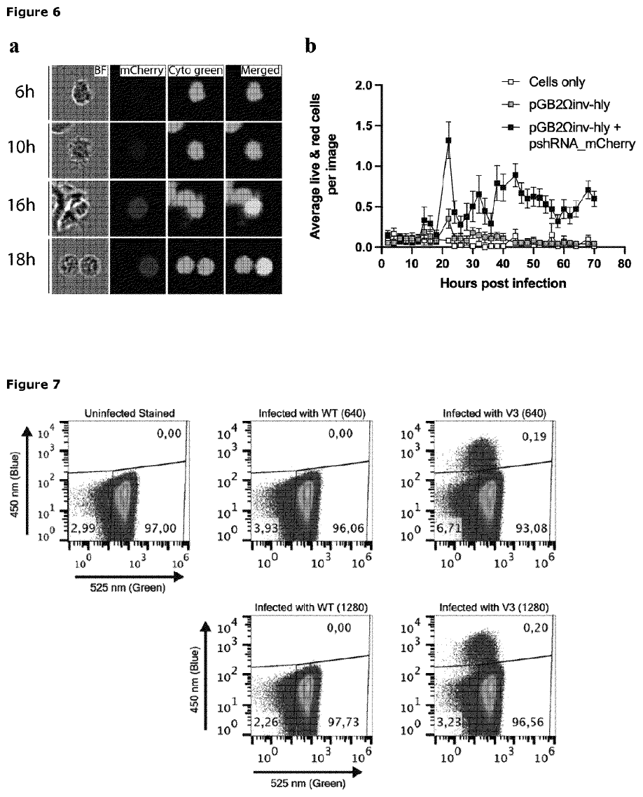

FIG. 6a follows the faith of an infected cell over time. The cell slowly started to exhibit mCherry expression 10 hours post infection followed by a rapid increase in fluorescence at 18 hours post infection. The live cell dye Cytolight Rapid Green was furthermore used to confirm cell health. The image at 18 hours post infection provides a clear example of two cells with identical healthy cellular morphology and bright staining with the live cell dye but only one cell showing expression of the mCherry reporter. Quantification of average live and red cell numbers per image over time is shown in FIG. 6b. Numbers of live and red cells were significantly higher for cells infected with invasive bacteria carrying the shRNA plasmid when compared to the negative controls of uninfected or invasive bacteria infected cells. mCherry expressing cells could be quantified for more than 70 hours post infection. In addition to providing evidence for a prove of concept for DNA delivery in form of the mCherry protein expression, this experiment also demonstrated the functional delivery of the therapeutic anti CD3d shRNA which was contained on the same delivered plasmid as a fusion to mCherry.

### Example 5 Recombinant Bacteria-Mediated Delivery Vectors (Inv-Hly) to Transfer Proteins into T-Cells

E. coli Nissle-pMUT1 containing the inv-hly expression plasmid pV3, encoding a b-lactamase enzyme, was used to infect Jurkat E6-1 cells, to demonstrate the use of the engineered E. coli to transfer of a reporter protein to an infected T-cell.

Methods:

Jurkat E6-1 cells were diluted to 2.2×10⁶ cells/ml in pre-warmed RPMI+10% FCS at 5 ml per flask. Overnight cultures of EcN+pV3 were diluted in complete cell culture medium at MOIs ranging from 640-1280 or 1.4×10⁹-2.8×10⁹ cfu/ml. WT EcN infected cells or uninfected cells served as negative controls for protein transfer. 5 ml of bacterial dilutions were added to the cells and culture flasks were incubated for 2 hours at 37° C. and 5% CO₂. To terminate cell infections, flask contents were transferred to 50 ml falcon tubes (Corning) and washed once with PBS. Washed cell pellets were resuspended in complete culture medium containing 50 μg/ml Gentamicin and transferred to new 50 ml suspension culture flasks. Cell cultures were incubated for 2-24 hours at 37° C. and 5% CO₂. In order to detect β-lactamase protein transfer, cultured cells were labelled with the LiveBLAzer™ FRET-B/G Loading Kit with CCF4-AM (Thermo Fischer Scientific), following the manufacturer's instructions. A modified Jurkat optimized loading protocol was used, after correspondence with Thermo Fischer Scientific. Specifically, a sort buffer consisting of Calcium- and Magnesium-free PBS, 1% glucose, 1 mM EDTA, and 1 mM HEPES was used instead of solution C from the LiveBLAzer kit. Labelled Jurkat cells were centrifuged to remove supernatants and resuspended in 1 ml of sort buffer for immediate flow cytometry analysis. Prepared cell suspensions were analysed on a Sony SH800S FACS.

Results:

As seen in FIG. 7a, FSC vs SSC populations were highly similar between infected and uninfected cells, which was indicative of healthy cell morphologies. Blue fluorescence of cleaved CCF4-AM was detected in 0.19% and 0.20% of cells infected with EcN pV3 at MOIs of 640 and 1280, respectively, which demonstrated successful protein transfer to T cells FIG. 7b. These results showed for the first time that functional protein could be delivered to non-phagocytic human immune cells using invasive bacterial delivery vectors.

### Example 6 Use of a Recombinant Bacteria-Mediated Delivery Vector (Inv-Hly) to Mediate Delivery and Translation of CD3d siRNA to T-Cells

Engineered E. coli strains (EcNΔdapA) containing the inv-hly expression plasmid (pGB2) and a vector comprising a recombinant nucleic acid molecule encoding an siRNA for silencing CD3d expression are used to infect T-cells (e.g. Jurkat) in vitro. CD3d siRNA translation in the infected cells serves to demonstrate that the engineered E. coli of the invention can be used for both transfer and functional translation of CD3d siRNA in mammalian T-cells. Such T cells, silenced in their CD3d expression by the delivered siRNA, confer a therapeutic effect in mouse models exhibiting TNP-KLH induced colitis, experimental allergic encephalomyelitis (EAE), and collagen-induced arthritis.

Methods

Plasmid construction: Plasmid pshRNA-CD3d (HSH022212-mU6-a-CD3D, Genecopoeia), is a non-viral shRNA expression vector for expression in mammalian cells that encodes a siRNA against human CD3d under the mammalian U6 promoter and SV40 poly A termination signal. Plasmid psiRNA-CD3d that allows transcription of the siRNA gene in a bacterial cell, was derived from pshRNA-CD3d by replacing its mammalian U6 promoter with a T7 promoter and inserting a T7 terminator downstream of the siRNA coding sequence. Plasmid pCS6 encoded a T7 RNA polymerase under control of an L-arabinose-inducible bacterial araBAD promoter (Addgene plasmid #55752).

Cells of E. coli strain EcNΔdapA were transformed with the plasmids: pshRNA-CD3d; psiRNA-CD3d; pCS6; or CSHCTR001-mU6, in combination with the inv-hly expression plasmid pGB2 (Table 4).

In vitro RNA transfer of siRNA: Jurkat E6-1 cells were infected in 6-well plates with EcNΔdapA strains comprising plasmids: pGB2, pCS6 and psiRNA-CD3d; or pGB2 and psiRNA-CD3d as a first negative control; or pGB2 alone as a second negative control, at a range of different MOIs, as described for in vitro DNA transfer. As a positive control, Jurkat cells were electroporated with anti-CD3d siRNA and incubated without bacteria. After infection, cells were labelled with anti-CD3d antibody and DAPI nuclear stain and analysed for CD3d silencing on both a flow cytometer and a fluorescent microscope.

In vivo transfer of siRNA: In vivo bacterial transfer of anti-CD3d siRNA into T cells was performed on members of mouse models having TNP-KLH induced colitis or EAE, or collagen-induced arthritis as previously described (Kuhn & Weiner, 2016). Members of each mouse model were injected i.v. with overnight cultures of invasive EcNΔdapA (pGB2) strains in PBS comprising the anti-CD3d siRNA plasmids (psiRNA-CD3d and pCS6); or invasive EcNΔdapA (pGB2) strains without siRNA plasmids as negative controls, or a commercial anti-CD3 antibody as a positive control. Treated mice were analysed for disease model specific markers as follows: for members of the TNP-KLH induced colitis model by daily tail vein and end point blood puncture blood samples to determine bacterial load, cytokine levels, and CD3 expression on target T cells via flow cytometry, histopathological evaluation of inflamed tissues, survival, and immunohistochemistry; for members of the EAE mouse model be daily tail vein and end point blood puncture blood samples to determine bacterial load, cytokine levels, and CD3 expression on target T cells via flow cytometry, measurement of pro-inflammatory cytokines in end point samples of brain tissue and spinal cord fluid, and survival; and for members of the collagen-induced arthritis mouse model by daily tail vein and end point blood puncture blood samples to determine bacterial load, cytokine levels, C-reactive protein (CRP), and CD3 expression on target T cells via flow cytometry, measurement of paw volume or thickness over time, and erythrocyte sedimentation rate.

### Example 7 Use of a Recombinant Bacteria-Mediated Delivery Vector (Inv-Hly) to Mediate Delivery and Expression of CRISPR/gRNA for Targeting Human Programmed Death-1 PD-1 Receptor in T-Cells

Engineered E. coli strains (EcNΔdapA) containing the inv-hly expression plasmid (pGB2) and a vector comprising a recombinant nucleic acid molecule encoding Cas9 and a gRNA sequence are used to infect T cells and target and knock-out the human PD-1 receptor (hPDCD1) gene on chromosome 2 Exon2, in vitro and in vivo. Knockout of hPD1 expression in the infected T cells serves to demonstrate that the engineered E. coli of the invention can be used for both transfer and functional expression of Cas9-gRNA-hPD1 in mammalian T-cells. Such T cells, modified to express Cas9-gRNA-hPD1, will confer a therapeutic effect on colorectal carcinomas in mice.

Methods

Plasmid construction: Plasmid pCas9-gRNA-hPD1, synthesised and cloned in a pUC57 backbone (Genscript), encodes a Cas9 endonuclease flanked by a SV40 Nuclear Localization Sequence (NLS) and a nucleoplasmin NLS and is operably linked to the bacterial J23105 promoter. The NLS increases the efficiency of nuclear localisation of the endonuclease protein and subsequent genome editing. The plasmid also encodes a gRNA sequence that targets the human PD-1 receptor (hPDCD1) on chromosome 2 Exon2 with the PAM sequence GGG. The gRNA is operably linked to the bacterial J23119 promoter which was modified to end with a SpeI site (Registry of Standard Biological Parts BBa_J23119). The plasmid was transformed into EcNΔdapA together with the inv-hly expression plasmid pGB2 (Table 4). As a positive control for bacterial transfer experiments, the gRNA from pCas9-gRNA-hPD1 was cloned into the gRNA backbone of plasmid pSpCas9(BB)-2A-GFP (Addgene plasmid #48138), which contains a Cas9 operably linked to a mammalian CMV promoter and enhancer control, a C-terminal fusion to EGFP, and a gRNA scaffold operably linked to a mammalian U6 promoter control, via Gibson assembly to create pCas9-GFP-gRNA-hPD1 [SEQ ID No.: 197].

In vitro CRISPR knockout of hPDCD1: Freshly isolated, primary, human T cells were activated with ImmunoCult™ Human CD3/CD28 T Cell Activator (Stemcell technologies) according to the supplier's instructions. In short, 10\hat{ }6 cells were seeded into freshly prepared ImmunoCult™-XF T Cell Expansion Medium (Stemcell technologies) containing Human Recombinant IL-2 (Stemcell technologies) and activated for 3 days at 37° C. and 5% CO₂ with ImmunoCult™ Human CD3/CD28 T Cell Activator antibodies.

Bacterial transfer of the above plasmids was performed in a flat-bottom 6-well plate, seeded with 1.2×10⁶ activated primary T cells per well. Cells were infected with overnight cultures of EcNΔdapA strains comprising plasmids: pGB2 and pCas9-gRNA-hPD1; or pCas9-gRNA-hPD1 alone as a first negative control; or pGB2 alone as a second negative control. Bacteria were added at a multiplicity of infection (MOI) of 500, 1000, or 2000 in ImmunoCult™-XF T Cell Expansion Medium media supplemented with IL-2. Additionally, primary T cells electroporated with pCas9-GFP-gRNA-hPD1 were seeded into bacteria-free wells as positive controls. Plates were centrifuged at 100×g for 10 min in a swinging bucket centrifuge, to initiate contact between cells and bacteria, and incubated for 1 hour at 37° C. and 5% CO₂. Next, well contents were transferred to individual 15 ml falcon tubes and washed trice with Phosphate Buffered Saline (Gibco™ PBS, pH 7.4, Fischer Scientific) at 300×g for 5 min at RT. The pellet was re-suspended in ImmunoCult™-XF T Cell Expansion Medium supplemented with IL-2 plus gentamicin to kill extracellular bacteria and further cultured for 24 hours at 37° C. and 5% CO₂ in an Incuyte S3 live imaging machine. After 24 hours, the cells were washed once in PBS at 300×g for 5 min. To determine hPD1 knockout, cells were incubated with recombinant anti-CD3d antibody (ab208514) and anti PD-1 antibody (ab52587, Abcam) and analysed on a flow cytometer. A sample aliquot was also analysed on a fluorescent microscope for visual confirmation of flow cytometer results.

To determine hPD1 knockout T cell activity, the infected activated T cells were re-suspended in ImmunoCult™-XF T Cell Expansion Medium supplemented with IL-2 and co-cultured with the PD-L1⁺ human breast cancer cell line MCF-7 (Y. Zheng et al., 2019) in a flat-bottom 6-well plate at a seeding density 6×10⁵ at a ratio of 1:1. Co-cultures were incubated in the presence of IncuCyte® Cytotox Red Reagent for counting of dead cells. The co-cultures were monitored at 37° C. and 5% CO₂ in an Incuyte S3 machine and analysed for an increase in red fluorescent signal as an indicator for T cell activity.

In vivo CRISPR knockout of hPDCD1: A group of CB6F1 mice were injected subcutaneously in the left and right flank with PD-L1 expressing CT26 murine colorectal carcinoma cells (ATCC CRL-2638). After 7 days, sub-groups of these mice were injected i.v. with overnight cultures of EcNΔdapA strains in PBS comprising of plasmids: pGB2 and pCas9-gRNA-hPD1; or pCas9-gRNA-hPD1 alone as a first negative control; or pGB2 alone as a second negative control, or PBS as a third negative control. An additional sub-group of mice were injected with Anti-mouse/human PD-L1 antibody (ab238697, Abcam) alone as a positive control. Body weight, temperature, survival, and food intake was measured daily to monitor signs of morbidity. Tumor size was measured daily to assess the cytotoxic activity of hPD1 knockout T cells. Tail vein blood samples were taken daily for later flow cytometric analysis of PD-1 expression of circulating lymphocytes as well as plating on LB agar plates with appropriate supplements and selection for determination of bacterial load in the blood stream. All mice were terminated after 20 days when spleens and tumors were collected for flow cytometric analysis of circulating lymphocytes and tumor cells. Heart puncture blood was assessed for cytokine/chemokine levels.

### Example 8 Recombinant Bacteria-Mediated Delivery Vectors (Inv-Hly) to Transfer Therapeutic OspF Protein to Primary T Cells

Methods

Primary human activated pan T cells were diluted to 4×10⁵ cells/ml in pre-warmed ImmunoCult XF T Cell Expansion medium supplemented with 25 ng/ml of IL-2 and added to 96 well plates at 100 μl per flask. Overnight cultures of either TOP10, containing either the invasive plasmid pGB3 or the invasive and therapeutic plasmid pGB4, were diluted in complete cell culture medium to an MOI of 1000 or 4×10⁸ cfu/ml. 100 μl of bacterial dilutions were added to the wells and plates were incubated for 2 hours at 37° C. and 5% CO₂. Cell suspensions were then transferred to a 96 well V-bottom plate and washed once with 100 μl of PBS. Cell pellets were resuspended in 200 μl pre-warmed complete culture medium containing MycoZap Plus-CL (500×). Cells were transferred to a new 96 well F-bottom plate and incubated for up to 3 days. For sample collection, cells were pelleted in a 96 well V-bottom plate and a total of 8 wells per replicate sample were resuspended and pooled in a total volume of 100 μl of PBS. Pooled samples were pelleted, resuspended in 180 μl of PBS and 20 μl of Image-iT Fixative Solution (4%) for final dilution of 0.4% paraformaldehyde, and incubated for 7 minutes at RT. The cell suspension was centrifuged, resuspended in 200 μl of PBS and 0.5 μl eBioscience Fixable Viability Dye eFluor® 780, and incubated at 4° C. for 15 minutes. From this point onward, all steps were performed with the samples protected from light. Next, cells were pelleted and fixed in 100 ul Image-iT Fixative Solution (4%) for 7 min at RT. The fixed cells were centrifuged and permeabilized by incubation in 100 μl of ice-cold 90% methanol in PBS for 15 minutes at 4° C. Fixed and permeabilized samples were pelleted and resuspended in 100 μl stain buffer followed by incubation at 4° C. for 15 minutes. After centrifugation to remove supernatants, cells were resuspended in 93 μl stain buffer and labelled with 5 μl of Phospho-Erk1/2 (Thr202, Tyr204) PE labelled antibody (MILAN8R, Thermo Fischer Scientific) and 2 μl of Erk 1/2 AF488 labelled antibody (C-9, Santa Cruz Biotechnology). Following an overnight incubation at 4° C., labelled cells were washed trice with 100 μl stain buffer and incubated in 100 ul of PBS containing 100 μg/mL DNAse and 5 mM MgCl₂ for 30 minutes at RT. Next, cells were washed once with 100 μl PBS containing 5 mM MgCl₂, resuspended in 200 μl PBS containing 5 mM MgCl₂ and 50 μg/mL DNAse, and analysed on a Novocyte Quanteon flow cytometer.

Results

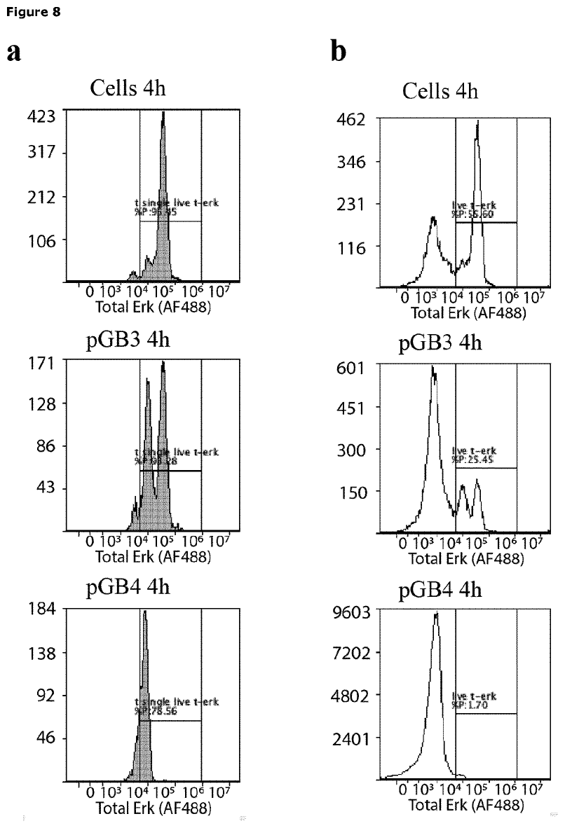

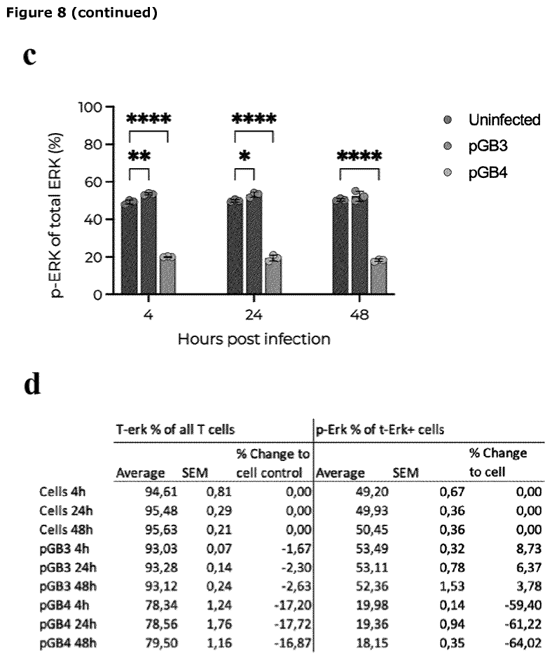

The MAPK phosphothreonine lyase OspF is a virulence factor of Shigella flexneri that has been shown to irreversibly dephosphorylate the human transcription factor extracellular signal-regulated kinase 1/2 (Erk). Erk dephosphorylation results in reduced TCR signaling, activation, and proliferation of T cells through inhibition of the MAPK/Erk pathway (Mattock & Blocker, 2017; Wei et al., 2012). OspF dephosphorylation of Erk has therefore high therapeutic potential in Erk-deregulated cancers such as Childhood Acute Lymphoblastic Leukaemia. As OspF was of bacterial origin and the mechanism of action in T cells was well understood, it was chosen for delivery to primary T cells using the inv-hly BACTERIAL INTRACELLULAR DELIVERY VECTOR system. Primary human activated T cells were infected with E. coli TOP10 carrying the therapeutic plasmid pGB4, which contained the inv-hly system as well as the therapeutic protein OspF. Uninfected cells or cells infected with bacteria carrying the invasive plasmid without OspF (pGB3) served as negative controls. Following 2 hours of infection, cells were cultured for up to 2 days in the presence of antibiotics and labelled with a viability dye as well as antibodies for total Erk (t-Erk) and phosphorylated Erk (p-Erk). Labelled cells were analysed on a flow cytometer to determine p-Erk percentages of infected cells. The gating strategy was as follows: All events>T cells>Single cells>live cells>t-Erk>p-Erk. The t-Erk positive populations were gated based on the uninfected cell controls where the smallest t-Erk peak was excluded and the majority of cells were included in the gate (FIG. 8a). Erk is an essential protein for a number of cellular processes such as proliferation, stress response, and differentiation. Accordingly, it would be expected that the majority of live T cells would express this crucial transcription factor. In addition, all live events were analysed for t-Erk (All events>live cells>t-Erk) to verify the t-Erk negative population (FIG. 8b). In this way, living bacteria and debris would also be included in the t-Erk histogram and could be used to verify the t-Erk negative gate, as bacteria and debris did not express Erk. Analysis of p-Erk percentages of live t-erk positive T cells revealed that cells infected with the therapeutic bacteria (pGB4) had a statistically significant reduction in p-Erk percentages, which suggested lower cell activation levels (FIG. 8c). p-Erk percentages were reduced by more than 60% when compared to the uninfected control cells (FIG. 8d). In contrast, cells infected with control bacteria (pGB3) had a statistically significant increase in p-Erk percentages. Nearly 10% higher percentages of p-Erk at 4 hours p.i. were observed before percentages returned close to control levels at 48 hours p.i. Comparison of t-Erk percentages of infected cells revealed that, while cells infected with control bacteria only had around 3% less t-Erk positive cells, pGB4 infected cells had around 15% less t-Erk positive cells than the uninfected control (FIG. 8d). Previous studies demonstrated that OspF reduced cellular p-Erk levels while leaving t-Erk levels unchanged (Li et al., 2007), and therefore it might be argued that the observed reduction in p-Erk percentages in cells infected with pGB4 bacteria was due to a reduction in overall t-Erk percentages and not due to the delivered OspF protein. However, this is not the case here, as a decrease in t-Erk percentages in pGB3 bacteria infected cells did not cause a decrease in p-Erk but rather showed an increase in p-Erk percentages, when compared to the uninfected control (FIG. 8d). The observation that cells infected with therapeutic bacteria did not simply return to control cell p-Erk percentages but rather decreased levels by more than 60% demonstrated the high potency of the delivered OspF therapeutic and thereby also the efficiency of the bacterial delivery system. Viabilities of pGB4 infected cells were lower than in the other experimental conditions, as indicated by lower percentages of t-Erk+live T cells (FIG. 8a). This decreased viability further validated the efficacy of OspF delivery, as this enzyme had been shown to irreversibly reduce p-Erk levels and therefore proliferation rates and viability.

Transfer of OspF also increased the percentage of live bacteria, shown as a stark decrease in the percentage of live cells that were t-Erk positive (FIG. 8b), as transfer of OspF reduced T-cell activation and thereby direct killing of bacteria lead to more viable bacterial cells that remained. As pGB3 bacteria did not carry the therapeutic they did not reduce cell activation, which resulted in more bacterial killing by activated T cells. As infected cells were grown in the presence of antibiotics, it was argued that detected live bacteria survived partly through spontaneous resistance mutations but largely due to adherence of bacterial cell clumps to the T cells, as previously observed microscopically, which sheltered bacterial cells from antibiotic exposure. Thus, increased bacterial survival presented an additional confirmation of successful therapeutic transfer. Lastly, primary T cells used for this study were activated for several days prior to infection. Consequently, infected cells were similar to activated T cells present in the human body during inflammatory disorders. The in vitro reduction of p-Erk levels in these activated cells by the therapeutic bacteria provides strong evidence for the potential therapeutic use of the BACTERIAL INTRACELLULAR DELIVERY VECTOR in inflammatory diseases in vivo.

In conclusion, this experiment showed, for the first time, that a bacterium engineered to express the inv-hly system can deliver a therapeutic protein at high efficacies to human T cells.

### Example 9 Use of a Recombinant Bacteria-Mediated Delivery Vector (Inv-Hly) to Mediate Delivery of NleB and NleE Proteins into T-Cells

Engineered E. coli strains (EcNΔdapA) containing the inv-hly expression plasmid (pV3) and a plasmid comprising a recombinant nucleic acid molecule encoding T3SS effectors s NleE and NleB are used to infect T-cells (e.g. NF-κB Reporter Jurkat) in vitro. Suppression of NF-kappaB activation in T-cells lines comprising an NF-κB Reporter serves to demonstrate that the engineered E. coli of the invention can transfer NleE and NleB proteins into the infected cells and interfere with the activation of selected host transcriptional regulators. Such T cells, whose host inflammatory pathways are manipulated by NleE/NleB-mediated suppression of NF-kappaB activation, confer a therapeutic effect in mouse models exhibiting TNP-KLH induced colitis, experimental allergic encephalomyelitis (EAE), and collagen-induced arthritis.

Methods

Plasmid construction: A DNA molecule comprising an operon encoding T3SS effectors NleE [SEQ ID No.: 219] and NleB [SEQ ID No.: 216] from the enteropathogenic E. coli 0127-H6 isolate EPEC E2348/69 was cloned into the pUC57-Kan plasmid (Genscript). The NleBE operon was operably linked to the strong constitutive promoter BBa_J23118 (Anderson library) and an optimised RBS sequence inserted before each gene. The predicted strength (EMOPEC) of the RBS upstream of NleE was higher than the RBS for the NleB gene to allow sufficient expression of both genes from a single promoter. The resulting plasmid, pNleBE was transformed into EcNΔdapA together with the inv-hly expression plasmid pV3 (Table 4).

In vitro NFκB inhibition: Bacterial transfer of the NleB and NleE proteins was performed in a flat-bottom 6-well plate, seeded with 1.2×10⁶ cells/well of the NF-κB Reporter (Luc)—Jurkat Cell line (Jurkat-Luc, BPS Bioscience) which comprises a firefly luciferase gene controlled by 4 copies of NF-kB response element upstream of a TATA promoter. Cells were infected with overnight cultures of EcNΔdapA strains comprising plasmids: pV3 and pNleBE; or pNleBE alone as first negative control; or pV3 alone as a second negative control. Bacterial cells of the EcNΔdapA strains were added at a multiplicity of infection (MOI) of 500, 1000, or 2000 in RPMI media supplemented with 10% fetal bovine serum (FBS). As a positive control, Jurkat cells were treated with 0.1 nM of the glucocorticoid triamcinolone acetonide (TA) (Tsaprouni, Ito, Adcock, & Punchard, 2007) alone. Plates were centrifuged at 100×g for 10 min in a swinging bucket centrifuge, to initiate contact between cells and bacteria, and incubated for 1 hour at 37° C. and 5% CO₂. Next, well contents were transferred to individual 15 ml falcon tubes and washed trice with Phosphate Buffered Saline (Gibco™ PBS, pH 7.4 at room temperature, Fischer Scientific) at 300×g for 5 min at RT. The pellet was re-suspended in complete growth medium plus gentamicin, to kill extracellular bacteria, and 10 ng/ml TNF-α was added for NFκB activation and subsequent stimulation of IL-8 production. After a 6-hour exposure to TNF-α, cells were washed thrice in RT PBS and re-suspended in complete growth medium plus gentamicin for 12 hours (overnight) at 37° C. and 5% s CO₂. Next, cells were centrifuged at 300×g for 5 min to collect cell supernatants. A 500 ul sample of the supernatant was used to determine IL-8 concentrations using a commercial ELISA kit for IL-8 (ELISA, human IL-8 Duo Set; R&D Systems, Minneapolis, MN, USA), according to the manufacturer's instructions. The remaining supernatant was used to re-suspend the cell pellets in their wells. 100 μl of ONE-Step™ Luciferase Assay reagent (BPS Bioscience) was added to each well and the plates were incubated for 30 minutes at RT followed by luminescence measurement in a luminometer to determine NFκB induced luciferase production.

In vivo NFkB inhibition: In vivo bacterial transfer of NleB and NleE proteins by invasive E. coli strains expressing an NleBE operon into T cells was performed on members of mouse models having TNP-KLH induced colitis or EAE, or collagen-induced arthritis as previously described (Kuhn & Weiner, 2016). Members of each mouse model were injected i.v. with overnight cultures of EcNΔdapA strains in PBS either comprising the plasmids: pV3 and pNleBE; or pNleBE plasmid alone as first negative control; or pV3 alone as second negative control. The treated mice are analysed for each of the disease model specific markers as described in example 6.

### Example 10 Use of a Recombinant Bacteria-Mediated Delivery Vector (Inv-Hly) to Mediate Delivery of L-Asparaginase into T-Cells

Engineered E. coli strains (EcNΔdapA) containing the inv-hly expression plasmid (pGB2) and a plasmid comprising a recombinant nucleic acid molecule encoding L-asparaginase II (ansB) are used to infect T-cells (e.g. the human acute leukemic T-cell lymphoblast, Jurkat E6-1) in vitro. Since acute leukemic T-cells lines cannot synthesize asparagine, asparagine starvation leads to their apoptosis and cell death. Hence death of acute leukemic T cells following contact with cells of the engineered E. coli strains, serves to demonstrate that they can transfer asparaginase II into the infected cells and cause asparagine starvation. When administered to a mouse model for acute leukemia, cells of the engineered E. coli strains may confer a therapeutic effect.

Methods

Plasmid construction: The L-asparaginase II gene ansB (NCBI Reference Sequence: NP_415200.1) [SEQ ID No.: 222] was cloned into a pUC57 plasmid operatively linked to the promoter BBa_J23100, an optimised RBS sequence (98.3% EMOPEC prediction), and a transcription terminator T1 from the E. coli rrnB gene. The constructed plasmid, pUC57-ansB, was transformed into EcNΔdapA together with the inv-hly expression plasmid pGB2 (Table 4).

In vitro L-asparaginase delivery: Bacterial transfer of L-asparaginase was performed in a flat-bottom 6-well plate, seeded with 1.2×10⁶ Jurkat E6-1 cells per well. Cells were infected with overnight cultures of EcNΔdapA strains comprising the plasmids: pGB2 and pUC57-ansB; or pUC57-ansB alone as a first negative control; or pGB2 alone as a second negative control. Bacteria cells of the EcNΔdapA strains were added at a multiplicity of infection (MOI) of 500, 1000, or 2000 in RPMI media supplemented with 10% fetal bovine serum (FBS). Additionally, Jurkat E6-1 cells were treated with commercially available L-asparaginase from Escherichia coli (Sigma Aldrich) as a positive control. Plates were centrifuged at 100×g for 10 min in a swinging bucket centrifuge, to initiate contact between cells and bacteria, and incubated for 1 hour at 37° C. and 5% CO₂. Next, well contents were transferred to individual 15 ml falcon tubes and washed trice with Phosphate Buffered Saline (Gibco™ PBS, pH 7.4 at room temperature, Fischer Scientific) at 300×g for 5 min at RT. The pellet was re-suspended in complete growth medium plus gentamicin, to kill extracellular bacteria, and Incucyte® Caspase-3/7 Green Reagent and IncuCyte® Cytotox Red Reagent was added to monitor apoptosis and cell death, respectively. Cells were incubated 37° C. and 5% CO₂ for 48 hours inside an Incucyte S3 live cell imaging machine.

In vivo L-asparaginase delivery: Five- to seven-week-old female NOD.Cg-Prkdcscid Il2rgtmlWjl/SzJ (NSG) mice (The Jackson Laboratory) (Mamonkin, Rouce, Tashiro, & Brenner, 2015) were intravenously injected with 3×10⁶ firefly luciferase expressing Jurkat E6-1 cells (Jurkat-FFluc). After 3- or 6-days post engraftment, mice were intravenously injected with overnight cultures of EcNΔdapA strains comprising the plasmids: pGB2 and pUC57-ansB; or pGB2 alone as a first negative control; or PBS without bacteria as a second negative control. As a positive control, mice were injected intraperitoneally with 6 U/g L-asparaginase (Sigma Aldrich) (Takahashi et al., 2017) 3- or 6-days post-engraftment. To monitor tumor burden, mice were injected intraperitoneally with D-Luciferin (150 ug/kg) and luminescence was determined with an IVIS Imaging system (Caliper Life Sciences). Daily tail vein blood samples were taken to determine tumor load via flow cytometry and to measure cytokine levels via ELISA.

### Example 11 Engineering Bacteria-Mediated Delivery Vectors Expressing a Gp120-Gp41-Antigen 43 (FLU) Fusion Protein

A bacteria-mediated delivery vector was engineered by transforming the deletion strain, EcN-pMUT1 ΔdapA with recombinant nucleic acid molecules encoding the envelope s glycoproteins gp120 and gp41 found in Human Immunodeficiency Virus 1 (HIV-1) and a third protein derived from a member of the 1.B.12.8.2 autotransporter-1 (at-1) family. When the three proteins are expressed as domains linked together in a fusion protein, they confer on the cell the ability to act as a bacteria-mediated delivery vector for delivery of the therapeutic agent to a mammalian non-phagocytic immune cell.

Methods:

Cloning “Gp120-Gp41-Antigen 43 (FLU)” Expression Plasmid:

The construction of a novel HIV env protein complex for bacterial expression was based on a modified protocol of Rathore et. al., In short, the sequence of HIV env protein mimic BG505 NFL.664 was chosen as a design template (Sarkar et al., 2018) due to favourable CD4 CCR5 cell targeting tropism and reduced glycosylation sites. The deleted membrane proximal external region (MPER) of the BG505 NFL.664 sequence was added back to maintain crucial membrane fusion functionality. The missing MPER sequence was added from uniprot entry Q2N0S6 which was used for annotation of the BG505 NFL.664 protein crystal structure 6B0N. In addition, the native amino acid, isoleucine, at position 559 was returned to maintain structural flexibility. To improve intracellular protein assembly and bacterial outer membrane expression, protein glycosylation was removed through mutation of asparagine (N) residues of N-linked glycosylation motifs (NXT/S) in the amino acid sequences of gp120 and gp41. The creators of the BG505 NFL.664 construct published identified glycosylation sites in their protein construct but were only able to detect glycans in the resolved crystal structure of the protein (Sarkar et al., 2018). The 6B0N protein sequence however showed stretches of sequence that contained NXS/T glycosylation motifs that lacked a secondary structure. In addition, glycosylation sites identified by the authors were described at incorrect AA positions due to incomplete numbering in the 6B0N PDB file stemming from breaks in secondary structure. Therefore, it was decided to mutate all NXS/T motifs in the sequence to further design the novel env protein complex. A total of 29 potential glycosylation sites were identified in the protein sequence, equal to the number identified in the reference HIV-1 strain HxB2. Using the NetNGlyc 1.0 Server, 21 of the 29 potential motifs were predicted to be likely glycosylated (Gupta & Brunak, 2002). After NXS/T motifs were identified, the tool HBPLUS was used to detect potential hydrogen bonds between Asn and neighbouring AA to avoid unsatisfied hydrogen bonding groups upon NXS/T mutations. AA frequencies at NXS/T motifs were identified from a multiple sequence alignment of 3978 HIV1 env sequences from a 2019 HIV-1 sequence compendium (Erk.hiv.lanl.gov) using the webtool AnalyzeAlign. The BG505 NFL.664 env AA sequence was used as a reference sequence with the following AA sequence added from the HxB2 reference sequence to the sequence beginning, to improve alignment numbering: MRVKEKYQHLWRWGWRWGTMLLGMLMICSATEK (SEQ ID NO.: 282). The ASN in the BG505 NFL.664 env sequence was then changed to the second most frequent AA at this position to remove glycosylation. The AA was changed to a less frequent AA if any unfavorable charge variations or structurally similar Ala and Gln were encountered. Using the software Chimera, the mutated sequence was computationally modelled against the original 6B0N sequence to determine any possible structural changes caused by AA mutations (Pettersen et al., 2004). Due to partially missing secondary structures, the 6B0N file was first modelled using Chimera to create a complete protein structure model. The structural similarity of the novel mutated env model with the refined 6B0N model was evaluated in Chimera based on the GA341 model score (Values higher than 0.7 generally indicate a reliable model with more than 95% probability of having the correct fold), the zDOPE normalized Discrete Optimized Protein Energy score (Negative values indicate better models), and the estimated RMSD model score (lower values indicate better models). The 2×GGGGS linker present in BG505 NFL.664 was maintained as it was previously determined to be of optimal length to allow native-like non-covalent bond formation and enable correct trimer formation (Sharma et al., 2015).

After confirming structural integrity of glycosylation site mutations, the E. coli outer membrane autotransporter Antigen 43 (flu) was added to the mutated protein sequence to enable bacterial surface expression. As the gp120 part of the env complex needed to be exposed away from the bacterial membrane and gp41 needed to be in close proximity of the bacterial membrane, anchoring could not be done through commonly used n-terminal fusion to an anchoring protein. Instead, the nucleotide sequence encoding the fused gp120-linker-gp41 domains was cloned between the coding sequences for the signal peptide and the N-terminus of the linker peptide of flu. The linker peptide ensured that the fused gp120-linker-gp41 proteins were displayed on the surface of the bacterial cell and not inside the R-barrel of the Flu protein where the Flu autochaperone C-terminus is located. The autotransporter domain serves to anchor the envelope complex to the bacterial cell membrane, and thereby performs the function of the hydrophobic transmembrane region of gp41, which was omitted from the fusion protein encoding gene construct. The resulting plasmid pCOLA_gp120-gp41-flu (SEQ ID NO 169) was transformed into E. coli TOP10. Bacteria were labelled with anti-HIV gp160 antibody (FITC conjugated, orb461521, Biorbyt) and observed on a Leica DM4000 B fluorescent microscope (Leica Microsystems) to confirm surface expression of gp160.
pGP140_Flu_hly plasmid: An optimized version of pCOLA_gp120-gp41-flu was constructed by inserting the Sau96I hly fragment from pSQ11 into the pCOLAgp120-gp41-flu plasmid, using standard Gibson assembly, to replace the lacI promoter and lac repressor gene region (6997-8157).

Results:

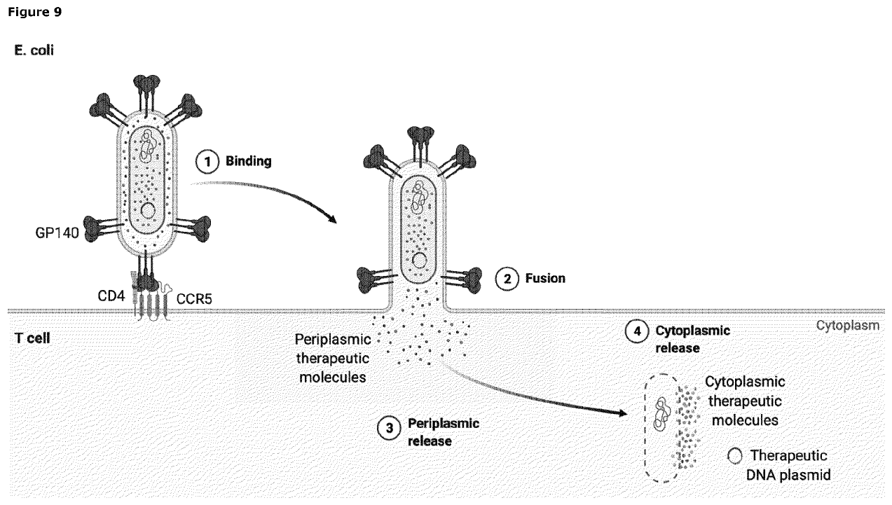

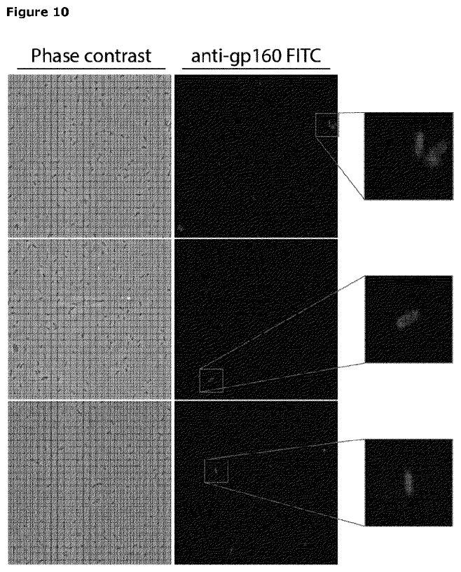

In this study, a novel bacterial intracellular delivery vector was constructed that exploits the natural CD4+CCR5+ cell specific targeting feature of the HIV-1 env protein complex gp120-gp41. Recombinant versions of this protein complex are termed gp140, not to be confused with the pre-cleavage complex gp160. In its native viral host, the gp120-gp41 complex initiates fusion of the viral capsid with the target cell membrane to enable virus entry into the host. In contrast to viruses and mammalian cells, gram negative bacteria contain both outer and inner membranes. Therefore, as demonstrated herein, expression of gp120-gp41 on the E. coli outer membrane surface allows for targeted delivery of periplasmic and/or cytoplasmic molecules to CD4+CCR5+ T cells in a novel manner, as illustrated in FIG. 9.

The design strategy used for the novel gp120-gp41 construct was based on the BG505 NFL.664 env 6B0N sequence (Sarkar et al., 2018). After the mutation of 29 potential glycosylation sites and addition of several missing AA sequences, the novel construct was modelled against the reference structure 6B0N to validate that original secondary structures were maintained. Using Chimera, the novel model was confirmed to be highly similar to the reference model with GA341, zDOPE, and estimated RMSD values of 1.00, −0.13, and 7.347, respectively. Some of the predicted alpha helixes in the gp120 model were slightly shifted compared to the reference model structure. The amino acid sequence at these areas was the same and it was therefore assumed not to result in any change in protein function. After cloning of the novel BACTERIAL INTRACELLULAR DELIVERY VECTOR gp120-gp41 env construct into an expression plasmid and transformation of the resulting pCOLAgp120-gp41-flu plasmid into E. coli TOP10, surface expression of BACTERIAL INTRACELLULAR DELIVERY VECTOR gp120-gp41 was investigated using a FITC labelled anti-HIV gp160 antibody. As shown in FIG. 9, several bacterial cells were stained with the gp160 antibody, with varying intensity. This staining confirmed the surface expression of the BACTERIAL INTRACELLULAR DELIVERY VECTOR gp120-gp41 complex. In order to increase expression further, the plasmid was transferred into the E. coli strain T7 (see further example herein), which was specifically designed to allow strong surface expression of complex protein structures.

### Example 12 Recombinant Bacteria-Mediated Delivery Vectors (Gp140) to Transfer Periplasmic Protein to Primary T Cells

Methods

Primary human activated pan T cells were diluted to 5×10⁵ cells/ml in pre-warmed ImmunoCult XF T Cell Expansion medium supplemented with 25 ng/ml of IL-2 and added to 48 well plates at 200 μl per flask. An overnight culture of either E. coli T7, carrying the invasive plasmid pCOLA-gp120-gp41 (SEQ ID NO. 169) and the periplasmic fluorescent protein reporter plasmid pSW002-Pc-TorA(sp)-mTurquoise2 (SEQ ID NO. 268), was diluted in complete cell culture medium to an MOI of 1280 or 6.4×10⁸ cfu/ml. E. coli T7+pCOLA-gp120-gp41 without the reporter plasmid as well as uninfected T cells served as negative controls. 200 μl of bacterial dilutions were added to the wells and plates were centrifuged at 100×g for 30 seconds to initiate contact between the bacteria and human cells. The co-cultures were incubated for 2 hours at 37° C. and 5% CO₂ to allow cell infections to occur. Next, cell suspensions were pelleted in 96 well V-bottom plates and resuspended in 200 μl of complete cell culture medium containing 50 μg/ml Gentamicin. Cell suspensions were transferred to new 48 well plates and incubated for a total of 6 hours. For sample collection, cells were pelleted in 2 ml Eppendord tubes and fixed in 1 ml of Image-iT Fixative Solution (4%) for 15 minutes at RT. Fixed cells were pelleted, resuspended in 100 μl PBS and transferred to a new 96 well plate for analysis on a Cytoflex S flow cytometer. 10 μl of each sample was stained with FITC conjugates anti-E. coli LPS antibody, mounted on microscopy slides and observed under a Leica DM4000 B fluorescent microscope

Results

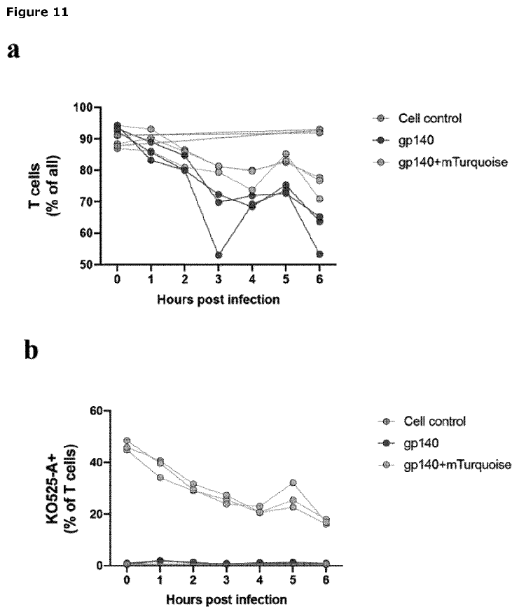

Infected cells were analysed for mTurquoise2 expression on a fluorescence microscope for visual detection and a flow cytometer for quantification. A differential antibody staining method was used for fluorescence microscopy where an anti-E. coli LPS antibody labelled extracellular bacteria and absence or presence of mTurquoise2 fluorescence indicated successful or unsuccessful protein delivery, respectively. Flow cytometry revealed that cell numbers decreased over time for the bacterial conditions, as expected due to the cells being exposed to bacterial endotoxins and nutrient depletion in the media during infection periods (FIG. 11a). In contrast, uninfected cell controls slightly increased in cell number over time. Nearly 50% of T cells infected with bacteria expressing gp140 and mTurquoise2 exhibited mTurquoise2 fluorescence at 0 h p.i., which decreased overtime. Other control conditions showed no mTurquoise2 signal at any timepoint sampled (FIG. 11b). While flow cytometry analysis seemed to indicate high rates of periplasmic protein transfer, fluorescence microscopy showed drastically different results. As seen in FIG. 11c, microscopy analysis revealed that the large majority of observed cells were surrounded by high amounts of mTurquoise2 positive bacteria and that this bacterial mTurquoise2 signal decreased overtime. Furthermore, no mTurquoise2 fluorescence was observed to originate from the T cells themselves.

This observation demonstrated that the majority of observed fluorescence from cells during flow cytometry analysis most likely originated from adherent bacteria and not from intracellular delivery to T cells. On the other hand, this observation also demonstrated the high efficacy of T cell binding by the gp140 protein complex. Considering the weak fluorescence of mTurquoise2 observed on microscopy images, fluorescent proteins, concentrated in the bacterial periplasm, could be highly diluted in their fluorescent signal upon release into the mammalian cell periplasm. This would likely result in a weak fluorescence signal within infected target cells which would be below the detection limit of fluorescence analysis from both microscopy and flow cytometry.

Despite this, microscopy analysis still revealed several cells with adherent bacteria that lacked mTurquoise2 fluorescence which indicated periplasmic protein transfer. The number of observed protein transfer events per cell increased over time, which indicated the high numbers of adherent cells could possibly translate into high periplasmic transfer events if infected cells were observed for more than 6 hours. Of note was an observation at the 5 hour time point where one non-adherent bacterial cell lacked mTurquoise expression (FIG. 11c, white circle). It was argued that due to the exposure to antibiotics in the media after infection, bacteria died and lysed overtime which released the mTurquoise signal from the periplasm into the media, thereby decreasing the signal associated to those bacterial cells. This hypothesis would also explain the consistent decrease over time in mTurquoise positive cells detected with flow cytometry. Hence, while periplasmic proteins theoretically are useful tools to study protein transfer specifically originating from the bacterial periplasm, microscopic analysis of this experiment revealed limitations of their use due to false positives of protein loss from lysing bacteria.

### Example 13 Recombinant Bacteria-Mediated Delivery Vectors (Gp140) to Transfer Secreted Protein to PBMCs

Methods

PBMCs isolated from buffy coats were diluted to 2.2×10⁶ cells/ml in ImmunoCult XF T Cell Expansion medium supplemented with 25 ng/ml of IL-2 and added to a 50 ml suspension culture flask (Cellstar, Greiner Bio-One) at 5 ml per flask. Overnight cultures of E. coli T7+pCOLAgp120-gp41+pUC19 were diluted in complete cell culture medium at MOIs ranging from 640-1280 or 1.4×10⁹-2.8×10⁹ cfu/ml. E. coli T7+pCOLAgp120-gp41 infected cells or uninfected cells served as negative controls for protein transfer. 5 ml of bacterial dilutions were added to the cells and culture flasks were incubated for 2 hours at 37° C. and 5% CO₂. To terminate cell infections, flask contents were transferred to 50 ml falcon tubes (Corning) and washed once with PBS. To detach adherent PBMC, a cell scraper was used to dislodge cells. Washed cell pellets were resuspended in complete culture medium containing MycoZap Plus-CL (Lonza Bioscience) and transferred to new 50 ml suspension culture flasks. Cell cultures were incubated for 2-24 hours at 37° C. and 5% CO₂. In order to detect β-lactamase protein transfer, cultured cells were labelled with the LiveBLAzer™ FRET-B/G Loading Kit with CCF4-AM (Thermo Fischer Scientific), following the manufacturer's instructions. In short, cells were centrifuged at 300×g for 7 minutes and resuspended in 6× loading solution containing CCF4-AM (solution A), solution B, and solution C/sort buffer, followed by incubation at RT for 1 hour under gentle shaking, protected from light. From this point onwards, cells were kept protected from light to avoid degradation of fluorochromes. Labelled primary cells were resuspended in 900 μl of PBS and 100 μl of Image-iT Fixative Solution (4%) for a final dilution of 0.4% paraformaldehyde. Cells were incubated for 7 minutes at RT, followed by centrifugation and resuspension in 999 μl of PBS and 1 μl of eBioscience Fixable Viability Dye eFluor® 780 (Thermo Fischer Scientific). After incubation at 4° C. for 15 minutes, cells were centrifuged and resuspended in 1 ml Image-iT Fixative Solution (4%). Following incubation at RT for 7 minutes, cells were washed once in 1 ml of PBS, resuspended in 500 μl stain buffer, and incubated for 15 minutes at 4° C. Next, cells were centrifuged and resuspended in 170 μl stain buffer and 10 μl each of anti-human CD8A SK1 APC antibody (Invitrogen, Thermo Fischer Scientific), anti-human CD3 OKT3 SB600 antibody (Invitrogen, Thermo Fischer Scientific), and anti-human CD4 OKT4 PE antibody (Invitrogen, Thermo Fischer Scientific). Cells were incubated with the antibodies overnight at 4° C. and the next day washed twice with 5 ml stain buffer. Antibodies and their dilutions are listed in table 5.

TABLE 5
antibodies

| AB type | Target antigen | Conjugate | Dilution | Ref number |
|---|---|---|---|---|
| Primary | human/mouse phospho-ERK1/2 | ebio pe | 1:20 | 12-9109-42 |
| Primary | human cd49d/integrin alpha 4) | — | 1:100 | 14-0499-82 |
| Primary | human erk 1/2 | af 488 | 1:50 | sc-514302 af488 |
| Primary | human cd3 | fitc | 1:20 | 555332 |
| Primary | e. coli serotype o/k | — | 1:100 | pa1-25636 |
| Primary | e. coli serotype o/k | fitc | 1:10 | pa1-73029 |
| Primary | hiv gp160 | FITC | 1:50 | orb461521 |
| Primary | anti-human CD8A | APC | 1:20 | 17-0087-42 |
| Primary | anti-human CD3 | SB600 | 1:20 | 63-0037-42 |
| Primary | anti-human CD4 | PE | 1:20 | 12-0048-42 |
| Secondary | mouse IgG1 | bv480 | 1:1000 | 746384 |
| Secondary | rabbit IgG | atto 550 | 1:1000 | 43328-1ml-f |

In an effort to remove cell aggregates in the suspension, cells were resuspended in 300 μl of PBS containing 100 μg/mL DNAse and 5 mM MgCl₂ and incubated for 30 minutes at RT. Cell suspensions were washed once with 1 ml of PBS containing 5 mM MgCl₂ and resuspended in 600 μl of PBS containing mM MgCl2 and 50 μg/mL DNAse. Finally, the cell suspension was gently passed through a pre-wet 70 μM reversible cell strainer (Stemcell Technologies) into 12×75 mm FACS tubes. Prepared cell suspensions were analysed on a Novocyte Quanteon flow cytometer. For compensation of fluorochrome spillover, single color control samples were prepared from cells infected with invasive bacteria carrying a β-lactamase expression plasmid. Auto-compensation was performed using the FlowLogic analysis software, where gates for positive populations as well as compensation values were manually adjusted to minimize fluorescence spillover. A compensation control for CCF4-AM was used to determine spillover into other channels from the green and blue signals as well as spillover from other fluorochromes into the detection filters for green and blue signals. As the CCF4-AM control emitted both green and blue fluorescence signals, no compensation was applied from the green signal in the blue detection filter and vice versa. The compensation matrix is shown in table 6.

TABLE 6
Compensation matrix for gp140 mediated secreted protein transfer to
primary lymphocytes.

| | CD8 | | CCF4-AM | CCF4-AM | CD3 | CD4 |
|---|---|---|---|---|---|---|
| | (APC) | FVS780 | (Blue) | (Green) | (SB600) | (PE) |
| CD8 (APC) | 100.00 | 7.60 | −0.10 | −0.10 | 0.05 | 0.03 |
| FVS780 | 0.59 | 100.00 | 0.07 | 0.15 | 0.04 | 0.36 |
| CCF4-AM | 0.09 | 0.05 | 100.00 | 0.00 | 0.45 | 1.10 |
| (Blue) | | | | | | |
| CCF4-AM | −0.01 | 0.00 | 0.00 | 100.00 | 2.09 | −0.20 |
| (Green) | | | | | | |
| CD3 (SB600) | 0.28 | −0.10 | 91.09 | 0.73 | 100.00 | 93.67 |
| CD4 (PE) | −0.60 | −0.20 | −9.00 | −17.50 | 2.16 | 100.00 |

Results

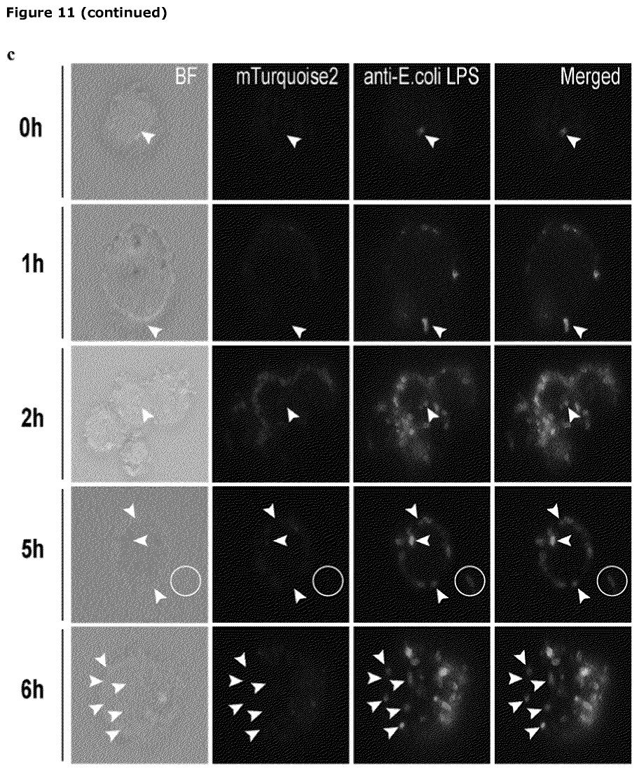

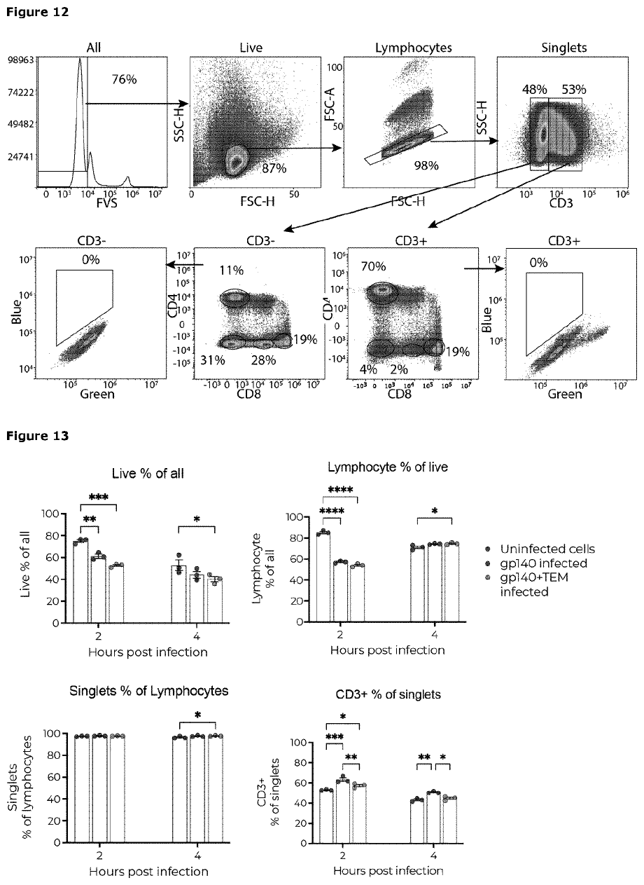

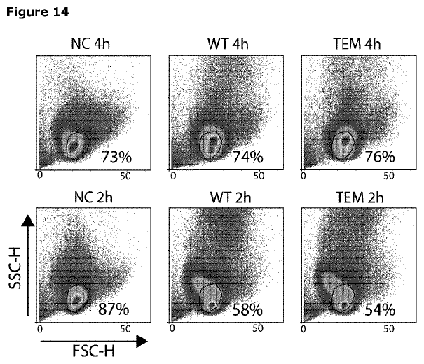

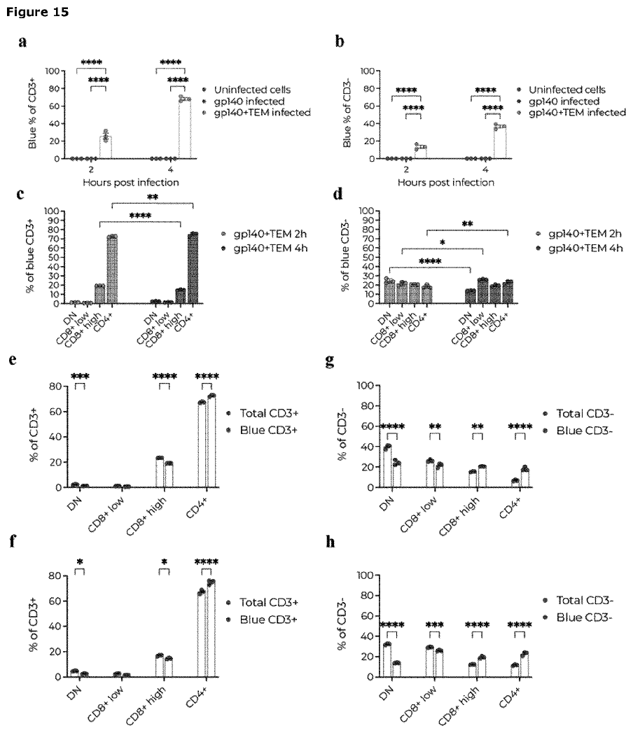

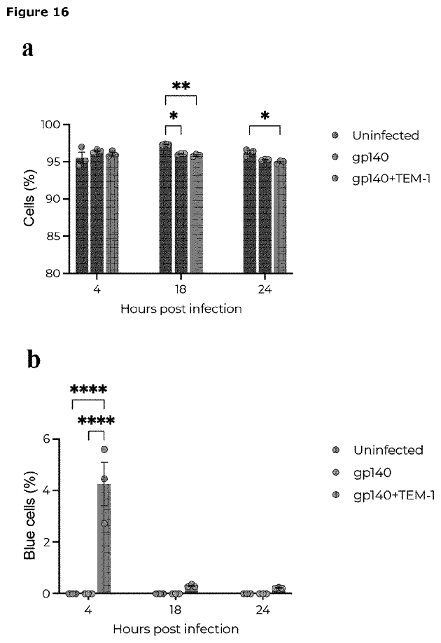

Cells were analysed on a flow cytometer with the CCF4-AM assay to detect protein transfer. In addition to CCF4-AM substrate loading, infected cells were also labelled with an antibody panel that allowed the identification of T and non-T cells with CD3 and further identification of cell subtypes with CD4 and CD8. A viability dye was used to exclude dead cells from analysis. After gating for live singlet lymphocytes, CD3+ and CD3− populations were assessed for their blue fluorescence and blue populations were further characterized by their distribution of CD4/CD8 positive sub populations (see FIG. 12). On average, cells infected with bacteria had significantly lower viabilities than uninfected controls but nearly recovered to control levels at 4 hours post infection (FIG. 13). This suggested that while an adverse effect of bacterial infection was present initially, it did not persist for more than 4 hours. The lymphocyte gate was designed to tightly include the major population of the uninfected control at 2 hours p.i. whilst still including the majority of events in the other conditions (see FIG. 14). Infected cells at 4 hours p.i. presented an emerging population of lower FSC and higher SSC. As this shift in size and complexity was characteristic of dying cells, these populations were excluded from the lymphocyte gate. Unfortunately, no monocyte populations could be identified. This lack of monocytes was likely due to the use of a T cell specific expansion media for culture which might have enriched the PBMC population for lymphocytes. CD3 populations were distinguishable at high resolution and CD3+ to CD3− ratios were slightly below the expected range for healthy human lymphocytes with 58% and 46% of CD3+ cells on average at 2 h and 4 h p.i., respectively, compared to the reference value of 70-85% (Kleiveland & Kleiveland, 2015) (FIG. 13). Cells infected with the control bacteria consistently had the highest percentages of CD3+ cells followed by cells infected with β-lactamase expressing bacteria. For CD3+ cells, CD4+:CD8+ratios were slightly higher than expected, with around 3:1 and 4:1 at 2 hour and 4 hour p.i., respectively (see FIG. 15e&f). CD3− populations consisted mainly of double negatives (see FIG. 15g&h). The second largest percentage was CD8+, with two distinct populations. Since no additional cell markers were used, it was not possible to conclude which cell type these populations represented. The blue gate, used to identify protein receiving cells, was designed to exclude all cells from the uninfected negative control. No compensation was performed for the CCF4-AM compound due to a lack of adequate single colour controls, however this lack of compensation did not affect the gating strategy, as any possible bleed trough from CCF4-AM green fluorescence into the blue detection channel and vice versa would not change blue populations. This was argued as follows: If β-lactamase delivery created more blue CCF4-AM through cleavage and the blue fluorescence would bleed into the green detection channel, then the cell population should increase in both blue and green fluorescence intensity. However, the intention of this experiment was to examine changes in blue fluorescence as an indicator for protein delivery. The only relevant consequence of bleed through could potentially occur from an increase in green CCF4-AM cleavage which would decrease the amounts of uncleaved green CCF4-AM and thereby reduce the bleed through of green fluorescence into the blue channel. This would reduce the blue population. In this case, increased cleavage events would also decrease bleed through into the blue channel which would improve the quality of results. An increase in blue fluorescence signal from bleed through of the green CCF4-AM compound could only be caused by an increase in green compound which was neither expected nor observed in this experiment. As illustrated in FIG. 16a, 25% and 68% of cells infected with E. coli T7+pCOLA-gp140+pUC19 were blue and CD3+ at the 2 hour and 4 hour timepoint, respectively, which indicated high rates of protein transfer. These percentages were significantly higher than what was observed during infections of isolated primary T cells with the same strain (4% on average at 4 h p.i.). This was likely due to a lack of a viability dye and T cell markers in the previous experiment which could have led to a proportion of events being falsely identified as live T cells and thereby increasing the percentage of non-blue cells. In addition, isolated T cells from earlier infection experiments were activated which could have resulted in increased granzyme-induced bacterial killing by cytotoxic T-lymphocytes and therefore reduced protein delivery rates.

A significantly smaller fraction of CD3− lymphocytes infected with E. coli T7+pCOLA-gp140+pUC19, around 37% at 4 h p.i., also appeared to have blue cells, indicative of bacterial protein transfer FIG. 16b. As mentioned previously, due to a lack of further cell markers no definitive conclusions could be made as to which cells types this blue population might represent. However, further examination of cell subtypes within the blue population further elucidated the nature of cells. In general, percentages of CD3 subtypes within the blue populations were highly similar to the percentages of the total CD3 populations. As expected, based on the mechanism of gp140 receptor binding, the majority of CD3+blue cells were CD4+(FIG. 15c). In contrast, while most subtypes were evenly represented at 2 h p.i., the majority of CD3− blue cells at 4 Erk p.i. were CD8+FIG. 15d. It is possible that these cells represented CD3− CD8+natural killer (NK) cells, which could have internalised bacteria via phagocytosis. The second largest subtype of CD3− blue cells were CD4+. These subtypes could possibly represent CD3− CD4+Lymphoid Tissue Inducer (LTi) cells, which are cell of the T cell lineage. LTi cells are believed to mainly play a role during embryogenesis in the formation of secondary lymphoid organs. In adults, LTi cells are known to secrete survival signals to adaptive and innate lymphoid cells and they have been found to play a role in inflammatory diseases such as psoriasis and rheumatoid arthritis. Binding of the gp120 protein to CD4 on LTi cells might have been sufficient to initiate internalization of bacteria and subsequent protein delivery.

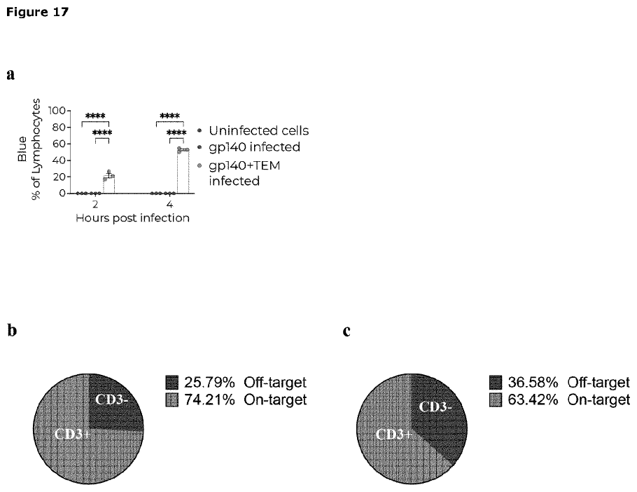

Given the highly similar distribution of CD3 cell subtypes within the blue CD3 population and the total CD3 population, one might argue that the majority of blue CD3+ cells being CD4+might not be due to specific gp140 targeting of the CD4 receptor but merely shows indiscriminate cell targeting that results in the initial cell distribution. If this argument was correct, then the percentage of CD4+ cells in the blue CD3+population should be the same as the CD4+percentage of the total CD3+population. For example, if the total CD3+population would consist of 70% CD4+ cells then the percentage of CD4+cells in the blue CD3+population should also be 70%. However, as seen in FIG. 15e&f, there were significantly higher percentages of CD4+ cells in the blue CD3+population than in the total CD3+population, around 5 and 7% more at 3 and 4 hours p.i., respectively. For the blue CD3− population, CD4+ and CD8+ cells were specifically targeted or specifically internalised the bacteria (FIG. 15g&h). It was also possible to evaluate the CD3 targeting efficiency of the gp140 system by analysis of CD3 expression of blue lymphocytes. Of the approximately 21% and 53% of lymphocytes that were blue roughly 74% and 63% were CD3+ at time points 2 hours and 4 hours, respectively (FIG. 17).

In conclusion, the results obtained in this study showed, for the first time, that both CD3 specific targeting and protein delivery by the bacterial gp140 delivery system occurred at high efficiencies.

### Example 14 Comparison of Injection Routes for Administration of Live Bacterial Delivery Vectors into Mice

Methods

Two different injection routes were evaluated for potential differences in tolerance to injected invasive bacteria in mice. Animal experiments were performed in house after approval from the Danish Animal Experiments Inspectorate. A total of 8 female CB6F1 mice, 6-8 weeks old, were housed in randomized pairs of four in Type3 cages inside a ScanTainer (Scanbur), with ad libitum access to water and regular chow diet (Altromin 1314, Altromin). Acclimatized animals were injected either intravenously or intraperitoneally with 100 μl of an overnight culture of EcNΔdapA, carrying invasive plasmid pSQ11, that was washed once in sterile PBS and diluted to 1×10⁹ cells/ml in sterile PBS. Immediately after injection, animals were closely monitored for changes in activity levels indicative of poor tolerance to bacterial injections. After 40 minutes, 4 hours and 1 week, 10 μl of blood were collected from the tail vein and stored in PBS on ice for later enumeration of viable bacterial cells. Immediately before and 7 days after bacterial injections, total body weight of the animals was measured. After 7 days, all animals were sacrificed through cervical dislocation and dissected to collect the liver, spleen, kidney, and lungs for later numeration of bacterial cells. The organs were weighted and transferred to gentleMACS C tubes (Miltenyi Biotec) containing 3 ml PBS for dissociation and generation of single cell suspensions using a gentleMACS Dissociator (Miltenyi Biotec). Dissociated liver, kidney, and lung samples were additionally passed through a 70 μm cell strainer. Single cell suspensions of prepared organs as well as tail vein blood samples were serially diluted in sterile PBS and plated on LB plates supplemented with DAP at a final concentration of 100 μg/ml and 50 μg/ml Kanamycin to enumerate live bacterial cell numbers.

Results

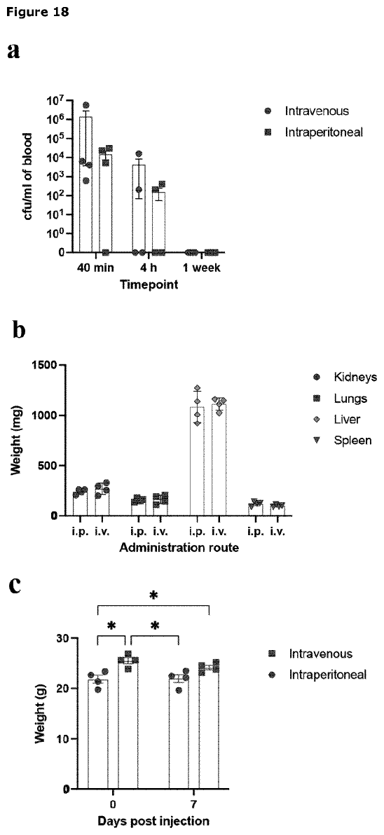

Only limited data exists on the safety of live EcN blood injections. Previous studies were all performed with non-auxotrophic E. coli strains which, in theory, were actively replicating after injection into the blood stream. In contrast, BACTERIAL INTRACELLULAR DELIVERY VECTORs designed in this study contained a DAP auxotrophy which inhibited growth in environments such as the bloodstream where DAP is not present. It was therefore hypothesised that higher amounts of auxotrophic bacteria could be safely injected into the bloodstream than what was deemed safe for non-auxotrophic replicating E. coli strains. It was first investigated whether a dose of 1×10⁸ cfu of auxotrophic EcN BACTERIAL INTRACELLULAR DELIVERY VECTOR could be administered safely into healthy mice via i.v. injection. In addition to i.v. injections, the intraperitoneal (i.p.) injection route was also investigated, as it was hypothesised that bacterial cells could be better tolerated through gradual release from the intraperitoneal cavity into the blood stream. Healthy, female, 6-8 weeks old CB6F1 mice were injected either i.v. or i.p with 1×10⁸ cfu/injection of the auxotrophic and invasive strain EcNΔdapA+pSQ11 and monitored over 1 week. Samples of tail vein blood and major organs were assessed for recovery of bacterial cells. Moreover, organ and body weights were measured to determine changes indicative of adverse reactions to BACTERIAL INTRACELLULAR DELIVERY VECTOR injections. As shown in FIG. 18a, bacteria could be recovered from blood for up to 4 hours p.i. with complete clearance after 1 week for both injection routes. I.v. injection lead to recovery rates of around 10⁶ cfu/ml blood on average at 40 minutes p.i. which was higher than for i.p. injections, as expected due to higher initial densities of bacteria in the blood. No bacteria were recovered from the kidneys, lungs, liver, or spleen at 1 week p.i. and all organs had normal weights for both injection routes (FIG. 18b). No change in total body weight was detected over 1 week for either of the injection routes (FIG. 18c). In conclusion, 1×10⁸ cfu/injections of auxotrophic and invasive BACTERIAL INTRACELLULAR DELIVERY VECTOR were well tolerated in healthy mice with no significant difference between injection route.

### Example 15 Maximum Injection Dose in Mice and Rats

Methods

In an effort to determine the highest possible dose of invasive bacteria that could be injected intravenously in a safe manner, rats and mice were injected in a dose escalating manner. A total of 10 female CB6F1 mice, 6-8 weeks old, or 10 female Sprague Dawley rats, 6-8 weeks old, were housed in randomized pairs of 2 in Type3 cages inside a ScanTainer (Scanbur), with ad libitum access to water and regular chow diet (Altromin 1314, Altromin). Overnight cultures of EcNΔdapA containing the invasive plasmid pGB3 were washed once in sterile PBS and diluted in sterile PBS to MOIs of 1030-1370 or 3×10¹⁰-4×10¹⁰ cells/ml for mouse injections, or MOIs of 29-98 or 3×10⁹-1×10¹⁰ cells/ml for rat injections. Either 100 μl or 500 μl of bacterial dilutions were injected intravenously into acclimatized mice or rats, respectively. Depending on cooperation, rats and mice were given low doses of the anaesthetic Hypnorm prior to i.v. injection. Changes in activity, measured in response to physical stimulation, were closely monitored immediately after injection as well as 30 minutes, 2 hours, 4 hours, and once daily after the first day for a total of 3 days. Total body weight was measured immediately before injections and once daily in the following days for a total of 3 days. Body temperatures were measured in triplicate, using a handheld infrared thermometer, immediately before injection as well as 30 minutes, 2 hours, 4 hours, and once daily after the first day for a total of 3 days.

Results

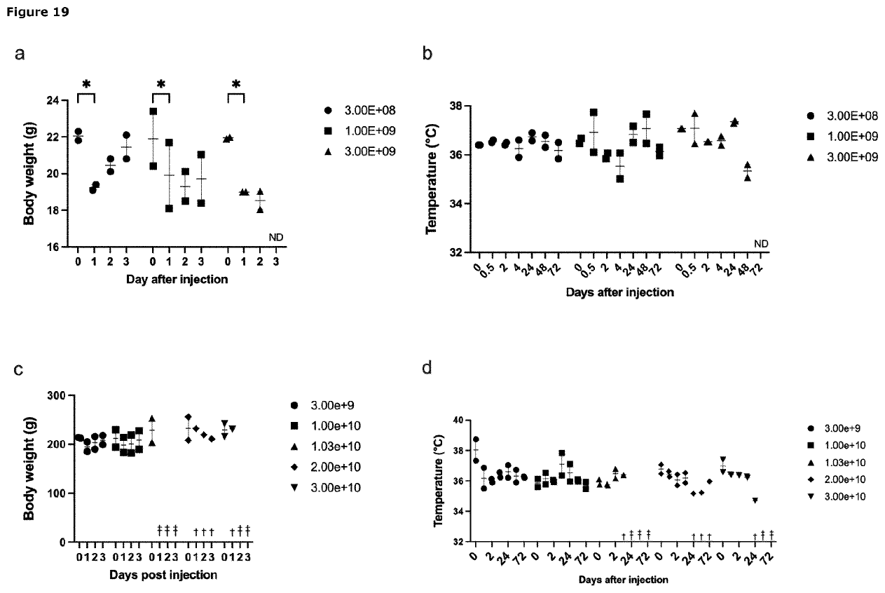

A range of 1×10⁸-3×10⁹ cfu of auxotrophic and invasive EcNΔdapA+pGB3 were injected i.v. into healthy CB6F1 mice and total body weight as well as body temperature was measured for 3 days p.i. Bacterial doses of up to 3×10⁹ cfu were tolerated with an initial drop in body weight after 1 day before slight recovery of body weights after 2 days when no statistically significant differences were observed compared to day 0 (FIG. 19a). The lowest temperature reached at this dose was 35.3° C. after 3 days compared to 37° C. before injection which was well within normal range (FIG. 19b). The body weight and temperature measurements on day 3 were unfortunately missed for the 3×10⁹ dose. Yet, visual inspection of these mice for up to 5 days p.i. indicated no further toxicity and these mice were as active in their foraging and grooming behavior as the mice receiving lower injection doses (Data not shown). Based on reference values for total blood volume and number of lymphocytes in mice, the highest tolerated dose of 3×10⁹ cfu translated to a 1027 MOI of bacteria to lymphocytes (Stemcell technologies Document #28048, National Centre for the Replacement Refinement & Reduction of Animals in Research). The same bacterial delivery vectors were also injected i.v. into female, 6-8 weeks old Sprague Dawley rats to determine the highest tolerated dose. Rats were injected with 3×10⁹-3×10¹⁰ cfu of auxotrophic and invasive EcNΔdapA+pGB3. Doses of up to 1×10¹⁰ cfu were well tolerated by rats with both total body weight and temperature recovering completely to baseline levels after 3 days (FIG. 19c&d). Based on rat reference values for lymphocyte numbers and blood volume, the highest dose of 1×10¹⁰ cfu/injection translated into an MOI of around 97 (Taconic, National Centre for the Replacement Refinement & Reduction of Animals in Research.

In conclusion, both rats and mice tolerated high doses of i.v. injections with auxotrophic and invasive bacterial delivery vectors. While rats tolerated higher cfu/injection, mice tolerated higher MOIs. Thus, this study successfully showed for the first time that engineered bacterial delivery vectors can be safely injected into the blood stream of rodents which provides a solid foundation for further in vivo investigations into the functionality and efficacy of therapeutic molecule transfer to T cells in vivo.

### Example 16 Use of Recombinant Bacteria-Mediated Delivery Vectors Expressing Invasin and Listeriolysin O to Transfer Genes into a Hela Cell Line

Engineered E. coli strain (EcN-ΔdapA) containing a combination of an inv-hly expression plasmid (pSQ11) and a reporter plasmid (PL0017; Example 1) containing a mCherry gene encoding monomeric red fluorescent protein (mCherry) was used to infect Hela cells and shown to additionally transfer and express the mCherry reporter gene in infected Hela cells.

Methods

HeLa cells were seeded in a 6-well plate at 1×10⁵ cells/well and allowed to attach overnight. Monolayers where then infected with cells of engineered E. coli EcNΔdapA pSQ11PL0017, or EcNΔdapA pSQ11 strains (Table 4) at an MOI of 500 for 1 hour at 37° C. and 5% CO₂. After the infection, monolayers were washed trice with PBS to remove bacteria and incubated in fresh DMEM media supplemented with 10% FCS and Ciprofloxacin (10 μg/ml) inside an Incucyte live cell imager at 37° C. and 5% CO₂.

Results

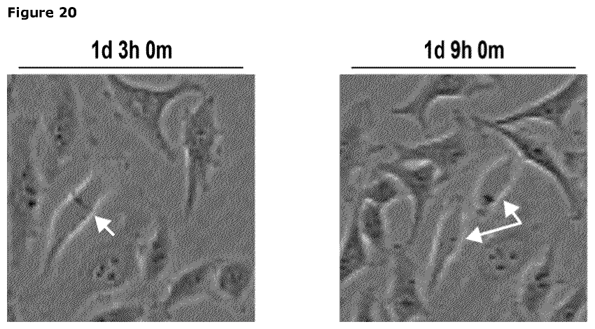

HeLa cells were infected with cells of engineered E. coli strains expressing the two-component delivery system (inv-hly) encoded by pSQ11, and comprising a mCherry reporter plasmid. Since mCherry gene is operably linked to a promoter and terminator functional in mammalian cells, its expression can only occur once the gene is transferred to the mammalian Hela cells by the invading E. coli and escapes into the Hela cell intracellular space. Detection of mCherry fluorescence in the infected Hela cells demonstrated that the E. coli cells invaded the Hela cells and transferred the mCherry reporter plasmid, while being absent in uninfected Hela cells or Hela cells infected with cells of E. coli strain pSQ11 plasmid (not shown). Expression of the transferred mCherry gene was first detected as fluorescence in the Hela cells after approximately 24 hours (FIG. 20).

### REFERENCES

Allenspach, E., Rawlings, D. J., & Scharenberg, A. M. (1993). X-Linked Severe Combined Immunodeficiency. In GeneReviews®. Retrieved from http://www.ncbi.nlm.nih.gov/pubmed/20301584
Anderson. C., 2006. Promoters/Catalog/Anderson—parts.igem.org. Regist Stand Parts. http://parts.igem.org/Promoters/Catalog/Anderson. Lee-Chang, C. (2013). Inhibition of Lung Metastasis by Chemokine CCL17-mediated In Vivo Silencing of Genes in CCR4+ Tregs. Journal of Immunotherapy, 36(4), 258-267.
Bloemberg, D., Nguyen, T., MacLean, S., Zafer, A., Gadoury, C., Gurnani, K., Doi, Y., Oki, S., Ozawa, T., Hohjoh, H., Miyake, S., & Yamamura, T. (2008). Orphan nuclear receptor NR4A2 expressed in T cells from multiple sclerosis mediates production of inflammatory cytokines. Proceedings of the National Academy of Sciences of the United States of America, 105(24), 8381-8386.
Bonde M T, Pedersen M, Klausen M S, Jensen S I, Wulff T, Harrison S, Nielsen A T, Herrg∪rd M J, Sommer M O A. 2016. Predictable tuning of protein expression in bacteria. Nat Methods 13:233-236.
Chen Y et al., 2019 Immunotherapy Deriving from CAR-T Cell Treatment in Autoimmune Diseases: J Immunology Research. Article ID 5727516 doi.org/10.1155/2019/5727516].
Chowdhury S, Castro S, Coker C, Hinchliffe T E, Arpaia N, Danino T, Tsang M, Gantchev J, Ghazawi F M, Litvinov I V. 2017. Protocol for adhesion and immunostaining of lymphocytes and other non-adherent cells in culture. Biotechniques 63:230-233.
Courvalin et al 1995: Transfert De Genes Des Bacteries Aux Cellules De Mammiferes. Comptes Rendus de l'Academie Des Sciences—Serie III, 318(12), 1207-1212. Retrieved from ncbi.nlm.nih.gov/pubmed/8745635
Croxen, M. A., Law, R. J., Scholz, R., Keeney, K. M., Wlodarska, M., & Finlay, B. B. (2013, October 1). Recent advances in understanding enteric pathogenic Escherichia coli. Clinical Microbiology Reviews, Vol. 26, pp. 822-880. https://doi.org/10.1128/CMR.00022-13
Dudda, J. C., Salaun, B., Ji, Y., Palmer, D. C., Monnot, G. C., Merck, E., Romero, P. (2013). MicroRNA-155 is required for effector cd8+t cell responses to virus infection and cancer. Immunity, 38(4), 742-753
Ebina, H., Misawa, N., Kanemura, Y., & Koyanagi, Y. (2013). Harnessing the CRISPR/Cas9 system to disrupt latent HIV-1 provirus. Scientific Reports, 3. https://doi.org/10.1038/srep02510
Flinn, A. M., & Gennery, A. R. (2018, April 24). Adenosine deaminase deficiency: A review. Orphanet Journal of Rare Diseases, Vol. 13, p. 65.
Fraietta, J. A., Lacey, S. F., Orlando, E. J., Pruteanu-Malinici, I., Gohil, M., Lundh, S., Melenhorst, J. J. (2018). Determinants of response and resistance to CD19 chimeric antigen receptor (CAR) T cell therapy of chronic lymphocytic leukemia. Nature Medicine, 24(5), 563-571
Freeley, M., & Long, A. (2013). Advances in siRNA delivery to T-cells: Potential clinical applications for inflammatory disease, cancer and infection. Biochemical Journal, Vol. 455, pp. 133-147
Gómez-Valadés, A. G., Llamas, M., Blanch, S., Perales, J. C., Roman, J., Gómez-Casajús, L., & Mascaró, C. (2012). Specific Jak3 downregulation in lymphocytes impairs y c cytokine signal transduction and alleviates antigen-driven inflammation in vivo. Molecular Therapy—Nucleic Acids, 1(9), e42.
Gupta, R., & Brunak, S. (2002). Prediction of glycosylation across the human proteome and the correlation to protein function. Pac Symp Biocomput, 310-322.
Gust, T. C., Neubrandt, L., Merz, C., Asadullah, K., ZOgel, U., & von Bonin, A. (2008). RNA interference-mediated gene silencing in murine T cells: In vitro and in vivo validation of proinflammatory target genes. Cell Communication and Signaling, 6(1), 3.
Hinterleitner, R., Gruber, T., Pfeifhofer-Obermair, C., Lutz-Nicoladoni, C., Tzankov, A., Schuster, M., Baier, G. (2012). Adoptive Transfer of siRNA Cblb-Silenced CD8+ T Lymphocytes Augments Tumor Vaccine Efficacy in a B16 Melanoma Model. PLoS ONE, 7(9), e44295.
Hou, P., Chen, S., Wang, S., Yu, X., Chen, Y., Jiang, M., Guo, D. (2015). Genome editing of CXCR4 by CRISPR/cas9 confers cells resistant to HIV-1 infection. Scientific Reports, 5. https://doi.org/10.1038/srep15577
Jorgensen, S. B., O'Neill, H. M., Sylow, L., Honeyman, J., Hewitt, K. A., Palanivel, R., Steinberg, G. R. (2013). Deletion of skeletal muscle SOCS3 prevents insulin resistance in obesity. Diabetes, 62(1), 56-64
Kuhn, C., & Weiner, H. L. (2016). Therapeutic anti-CD3 monoclonal antibodies: from bench to bedside. Immunotherapy, 8(8), 889-906
Kumar, P., Ban, H. S., Kim, S. S., Wu, H., Pearson, T., Greiner, D. L., Shankar, P. (2008). T Cell-Specific siRNA Delivery Suppresses HIV-1 Infection in Humanized Mice. Cell, 134(4), 577-586.
Lee, J., Yun, K. S., Choi, C. S., Shin, S. H., Ban, H. S., Rhim, T., Lee, K. Y. (2012). T cell-specific siRNA delivery using antibody-conjugated chitosan nanoparticles. Bioconjugate Chemistry, 23(6), 1174-1180.
Lee et al 2018: Modulatory upregulation of an insulin peptide gene by different pathogens in C. Elegans. Virulence, 9(1), 648-658. doi.org/10.1080/21505594.2018.1433969.
Li, H., Xu, H., Zhou, Y., Zhang, J., Long, C., Li, S., . . . Shao, F. (2007). The phosphothreonine lyase activity of a bacterial type III effector family. Science, 315(5814), 1000-1003. https://doi.org/10.1126/science.1138960
Li, R., Helbig, L., Fu, J., Bian, X., Herrmann, J., Baumann, M., Zhang, Y. (2019). Expressing cytotoxic compounds in Escherichia coli Nissle 1917 for tumor-targeting therapy. Research in Microbiology, 170(2), 74-79
Liao, H. K., Gu, Y., Diaz, A., Marlett, J., Takahashi, Y., Li, M., Belmonte, J. C. I. (2015). Use of the CRISPR/Cas9 system as an intracellular defense against HIV-1 infection in human cells. Nature Communications, 6. https://doi.org/10.1038/ncomms7413
Lovett-Racke, A. E., Rocchini, A. E., Choy, J., Northrop, S. C., Hussain, R. Z., Ratts, R. B., . . . Racke, M. K. (2004). Silencing T-bet defines a critical role in the differentiation of autoreactive T lymphocytes. Immunity, 21(5), 719-731.
Luheshi, N. M., Rothwell, N. J., & Brough, D. (2009). Dual functionality of interleukin-1 family cytokines: Implications for anti-interleukin-1 therapy. British Journal of Pharmacology, Vol. 157, pp. 1318-1329.
Lund, A. M., Kildegaard, H. F., Petersen, M. B. K., Rank, J., Hansen, B. G., Andersen, M. R., & Mortensen, U. H. (2014). A Versatile System for USER Cloning-Based Assembly of Expression Vectors for Mammalian Cell Engineering. PLoS ONE, 9(5), e96693. https://doi.org/10.1371/journal.pone.0096693 MacKay, M., Afshinnekoo, E., Rub, J., Hassan, C., Khunte, M., Baskaran, N., Mason, C.
E. (2020). The therapeutic landscape for cells engineered with chimeric antigen receptors. Nature Biotechnology, 38(2), 233-244
Mamonkin, M., Rouce, R. H., Tashiro, H., & Brenner, M. K. (2015). A T-cell-directed chimeric antigen receptor for the selective treatment of T-cell malignancies. Blood, 126(8), 983-992
Mattock, E., & Blocker, A. J. (2017). How do the virulence factors of shigella work together to cause disease? Frontiers in Cellular and Infection Microbiology, 7(MAR). https://doi.org/10.3389/fcimb.2017.00064
McComb, S. (2020). A High-Throughput Method for Characterizing Novel Chimeric Antigen Receptors in Jurkat Cells. Molecular Therapy—Methods and Clinical Development, 16, 238-254.
Moriwaki, A., Inoue, H., Nakano, T., Matsunaga, Y., Matsuno, Y., Matsumoto, T., Nakanishi, Y. (2011). T cell treatment with small interfering RNA for suppressor of cytokine signaling 3 modulates allergic airway responses in a murine model of asthma. American Journal of Respiratory Cell and Molecular Biology, 44(4), 448-455.
Müller, H. J., & Boos, J. (1998) Use of L-asparaginase in childhood ALL. Critical Reviews in Oncology/Hematology, Vol. 28, pp. 97-113
Nadler, C., Baruch, K., Kobi, S., Mills, E., Haviv, G., Farago, M., Rosenshine, I. (2010). The Type III Secretion Effector NleE Inhibits NF-κB Activation. PLoS Pathogens, 6(1), e1000743; https://doi.org/10.1371/journal.ppat.1000743
Novina, C. D., Murray, M. F., Dykxhoorn, D. M., Beresford, P. J., Riess, J., Lee, S. K., Sharp, P. A. (2002). siRNA-directed inhibition of HIV-1 infection. Nature Medicine, 8(7), 681-686. https://doi.org/10.1038/nm725
Pallandre, J.-R., Brillard, E., Crehange, G., Radlovic, A., Remy-Martin, J.-P., Saas, P., Borg, C. (2007). Role of STAT3 in CD4+CD25+FOXP3+Regulatory Lymphocyte Generation: Implications in Graft-versus-Host Disease and Antitumor Immunity. The Journal of Immunology, 179(11), 7593-7604.
Pettersen, E. F., Goddard, T. D., Huang, C. C., Couch, G. S., Greenblatt, D. M., Meng, E. C., & Ferrin, T. E. (2004). UCSF Chimera—A visualization system for exploratory research and analysis. Journal of Computational Chemistry, 25(13), 1605-1612. https://doi.org/10.1002/jcc.20084
Pollock, G. L., Oates, C. V. L., Giogha, C., Lung, T. W. F., Ong, S. Y., Pearson, J. S., & Hartland, E. L. (2017). Distinct roles of the antiapoptotic effectors NleB and NleF from enteropathogenic Escherichia coli. Infection and Immunity, 85(4).
Punj, V., Bhattacharyya, S., Saint-Dic, D., Vasu, C., Cunningham, E. A., Graves, J., Das Gupta, T. K. (2004). Bacterial cupredoxin azurin as an inducer of apoptosis and regression in human breast cancer. Oncogene, 23(13), 2367-2378
Qi, C., Li, D., Jiang, X., Jia, X., Lu, L., Wang, Y., Wei, M. (2018). Inducing CCR5 Δ32/A32 Homozygotes in the Human Jurkat CD4+Cell Line and Primary CD4+Cells by CRISPR-Cas9 Genome-Editing Technology. Molecular Therapy—Nucleic Acids, 12, 267-274.
Reisch C R, Prather K L J. 2015. The no-SCAR (Scarless Cas9 Assisted Recombineering) system for genome editing in Escherichia coli. Sci Rep 5.
Roberts, T. M., Rudolf, F., Meyer, A., Pellaux, R., Whitehead, E., Panke, S., & Held, M. (2016). Identification and Characterisation of a pH-stable GFP. Scientific Reports, 6(1), 1-9. https://doi.org/10.1038/srep28166
Sails H M. 2011. The ribosome binding site calculator, p. 19-42. In Methods in Enzymology. Academic Press Inc.
Sarkar, A., Bale, S., Behrens, A. J., Kumar, S., Sharma, S. K., De Val, N., . . . Wilson, I. A. (2018). Structure of a cleavage-independent HIV Env recapitulates the glycoprotein architecture of the native cleaved trimer. Nature Communications, 9(1), 1-14. https://doi.org/10.1038/s41467-018-04272-y
Self, M., Einsele, H., & Löffler, J. (2019) CAR T Cells Beyond Cancer: Hope for Immunomodulatory Therapy of Infectious Diseases. Frontiers in Immunology, Vol. 10, p. 2711
Sharma, S. K., deVal, N., Bale, S., Guenaga, J., Tran, K., Feng, Y., . . . Wyatt, R. T. (2015). Cleavage-Independent HIV-1 Env Trimers Engineered as Soluble Native Spike Mimetics for Vaccine Design. Cell Reports, 11(4), 539-550. https://doi. org/10.1016/j.celrep.2015.03.047
Silva-Rocha et al 2013: The Standard European Vector Architecture (SEVA): A coherent platform for the analysis and deployment of complex prokaryotic phenotypes. Nucleic Acids Research, 41(D1), D666. doi.org/10.1093/nar/gks1119
Su, S., et al., (2016). CRISPR-Cas9 mediated efficient PD-1 disruption on human primary T cells from cancer patients. Scientific Reports, 6(1), 1-14. https://doi.org/10.1038/srep20070
Takahashi, H., Inoue, J., Sakaguchi, K., Takagi, M., Mizutani, S., & Inazawa, J. (2017). Autophagy is required for cell survival under L-asparaginase-induced metabolic stress in acute lymphoblastic leukemia cells. Oncogene, 36(30), 4267-4276
Tesniere, A., Abermil, N., Schlemmer, F., Casares, N., Kepp, O., Pequignot, M., Kroemer, G. (2010). In vivo depletion of T lymphocyte-specific transcription factors by RNA interference. Cell Cycle, 9(14), 2902-2907
Tsaprouni, L. G., Ito, K., Adcock, I. M., & Punchard, N. (2007). Suppression of lipopolysaccharide- and tumour necrosis factor-α-induced interleukin (IL)-8 expression by glucocorticoids involves changes in IL-8 promoter acetylation. Clinical and Experimental Immunology, 150(1), 151-157.
Uribe-Quero, E., & Rosales, C. (2017, October 24). Control of phagocytosis by microbial pathogens. Frontiers in Immunology, Vol. 8, p. 24. https://doi.org/10.3389/fimmu.2017.01368
Wang, Z., et al., (2016). CRISPR/Cas9-Derived Mutations Both Inhibit HIV-1 Replication and Accelerate Viral Escape. Cell Reports, 15(3), 481-489
Wei, P., et al., (2012) Bacterial virulence proteins as tools to rewire kinase pathways in yeast and immune cells. Nature, Vol. 488, pp. 384-388
Wilton et al 2018: A New Suite of Plasmid Vectors for Fluorescence-Based Imaging of Root Colonizing Pseudomonads. Front Plant Sci. 2018 Feb. 1; 8:2242. doi: 10.3389/fpls.2017.02242. eCollection 2017. 10.3389/fpls.2017.02242 PubMed 29449848 (Addgene #111254)
Yang, S. C., Hung, C. F., Aljuffali, I. A., & Fang, J. Y. (2015, December 1). The roles of the virulence factor IpaB in Shigella spp. in the escape from immune cells and invasion of epithelial cells. Microbiological Research, Vol. 181, pp. 43-51. https://doi.org/10.1016/j.micres.2015.08.006
Zainuddin, H. S., Bai, Y., & Mansell, T. J. (2019). CRISPRQbased curing and analysis of metabolic burden of cryptic plasmids in Escherichia coli Nissle 1917. Engineering in Life Sciences, 19(6), 478-485. https:/doi.org/10.1002/elsc.201900003
Zheng, Y., Fang, Y. C., & Li, J. (2019). PD-L1 expression levels on tumor cells affect their immunosuppressive activity. Oncology Letters, 18(5), 5399-5407. https://doi.org/10.3892/ol.2019.10903
Zhu, W., et al., (2015). The CRISPR/Cas9 system inactivates latent HIV-1 proviral DNA. Retrovirology, 12(1). https://doi.org/10.1186/s12977-015-0150-z

1. An invasive recombinant bacterial cell for use in prevention and/or treatment of an immune-related disorder; said bacterial cell comprising one or more recombinant nucleic acid molecule(s) encoding one or more therapeutic agent(s) for use in prevention and/or treatment of said immune-related disorder in a mammal; wherein said bacterial cell comprises one or more recombinant invasive gene(s) that facilitates invasion and release of said one or more recombinant nucleic acid molecule(s) or said one or more therapeutic agent(s) in a mammalian non-phagocytic immune cell and thereby functions as a bacteria-mediated delivery vector for in vivo or ex vivo delivery of said one or more recombinant nucleic acid molecule(s) or said one or more therapeutic agent(s) to the mammalian non-phagocytic immune cell, and
wherein the immune-related disorder is selected from the group: an autoimmune disorder, cancer, and a lymphoproliferative disorder.

2. The invasive recombinant bacterial cell for use in prevention and/or treatment of an immune-related disorder according to claim 1; wherein said bacterial cell comprises one or more recombinant invasive gene(s) for expressing protein(s) selected from the group:
a. a 1.B.54 family invasin in combination with a 1.C.12.1.7 family cytolysin;
b. viral envelope glycoproteins, preferably comprising a HIV-1 glycoprotein 120 and a HIV-1 glycoprotein 41, in combination with a 1.B.12.8.2 family autotransporter-1; and
c. 1.C.36.3.1 secretion system proteins, preferably comprising Type III GPI-anchored ipaB and ipaC proteins.

3. The invasive recombinant bacterial cell for use in prevention and/or treatment of an immune-related disorder according to claim 1, wherein said therapeutic agent is a recombinant or native DNA, RNA, or protein agent selected from the group:
a. a Chimeric Antigen Receptor
b. a small interfering RNA
c. a protein inhibitor of any one of T cell activation; T cell suppression; T cell proliferation and T cell cell death;
d. a protein inducer of any one of T cell activation; T cell suppression; T cell proliferation and T cell cell death;
e. a cytotoxin
f. a cytokine
g. a chemokine, and
h. a CRISPR-Cas system.

4. The invasive recombinant bacterial cell for use in prevention and/or treatment of an immune-related disorder according to claim 1; wherein a mode of administration of said bacterial cell is selected from the group: intravenous, intra-arterial, intraperitoneal, intralymphatic, sub-cutaneous, intradermal, intramuscular, intraosseous infusion, intra-abdominal, oral, intratumor, intravascular, intravenous bolus; and intravenous drip.

5. The invasive recombinant bacterial cell for use in prevention and/or treatment of an immune-related disorder according to claim 1, wherein the bacterial cell is a species of the genus selected from the group: Escherichia, Bacteroides, Akkermansia, Alistipes, Prevotella, and Parabacteroides.

6. The invasive recombinant bacterial cell for use in prevention and/or treatment of an immune-related disorder according to claim 1, wherein said mammalian non-phagocytic immune cell is a T-lymphocyte, B-lymphocyte, Natural Killer cell, or Basophil.

7. The invasive recombinant bacterial cell for use in prevention and/or treatment of an immune-related disorder according to claim 1, wherein said mammalian non-phagocytic immune cell is a member of the group consisting of a primate, bovine, ovine, porcine, feline, buffalo, canine, goat, equine, donkey, and camel cell.

8. The invasive recombinant bacterial cell for use in prevention and/or treatment of an immune-related disorder according to claim 1, wherein said disorder is any disease which can be treated, prevented, ameliorated by modulating at least one component of the host immune system.

9. The invasive recombinant bacterial cell for use in prevention and/or treatment of an immune-related disorder according to claim 1, wherein said autoimmune disorder is selected from the group: Inflammatory bowel disease; Severe combined immunodeficiency; Organ transplant rejection (graft vs host disease); Asthma; Crohn's disease; Lupus nephritis; Autoimmune hepatitis; Alopecia Areata; Dermatitis; Dermatitis herpetiformis; Epidermolysis bullosa; Hidradenitis suppurativa; Psoriasis; Systemic scleroderma; Diabetes mellitus type 1; Ulcerative colitis; Autoimmune lymphoproliferative syndrome; Rheumatoid arthritis; Systemic lupus erythematosus; Multiple sclerosis; Primary immunodeficiency; and Pyoderma gangrenosum.

10. The invasive recombinant bacterial cell for use in prevention and/or treatment of an immune-related disorder according to claim 1, wherein cancer is selected from the group: Burkitt Lymphoma; Non-Hodgkin Lymphoma; Lymphocytic Leukemia; Myeloid Leukemia; Myelogenous Leukemia; Cutaneous T-Cell Lymphoma; Hodgkin Lymphoma; Multiple Myeloma; T-Cell Lymphoma, Acute lymphoblastic leukemia; Acute myeloid leukemia; Chronic lymphocytic leukemia; Chronic myelogenous leukemia; Cutaneous T-cell lymphoma; Diffuse large B-cell lymphoma; Follicular lymphoma; Hepatosplenic T-cell lymphoma; and Hairy cell leukemia.

11. The invasive recombinant bacterial cell for use in prevention and/or treatment of an immune-related disorder according to claim 1, wherein said lymphoproliferative disorder is selected from the group: post-transplant lymphoproliferative disorder; autoimmune lymphoproliferative syndrome; lymphoid interstitial pneumonia; Epstein-Barr virus-associated lymphoproliferative diseases; Waldenström's macroglobulinemia; Wiskott-Aldrich syndrome; Lymphocyte-variant hypereosinophilia; Pityriasis Lichenoides; and Castleman disease.

12. The invasive recombinant bacterial cell for use in prevention and/or treatment of an immune-related disorder according to claim 1, wherein said recombinant nucleic acid molecule(s) or said therapeutic agent(s) for use in prevention and/or treatment of said immune-related disorder is selected from:
a. a recombinant nucleic acid molecule(s) comprising DNA nuclear targeting sequence(s) for intra-nuclear localization,
b. a recombinant nucleic acid molecule(s) devoid of intra-nuclear targeting sequence(s),
c. a therapeutic agent comprising a protein comprising a nuclear localization sequence for intra-nuclear localization, or
d. a therapeutic agent comprising a protein devoid of intra-nuclear targeting sequences.

13. The invasive recombinant bacterial cell for use in prevention and/or treatment of an immune-related disorder according to claim 1, wherein said bacterial cell is a strain of E. coli, and wherein said one or more recombinant invasive gene(s) encode a 1.B.54.1.2 family invasin and a 1.C.12.1.7 family listeriolysin.

14. The invasive recombinant bacterial cell for use in prevention and/or treatment of an immune-related disorder according to claim 1, wherein said bacterial cell is a strain of E. coli, and wherein said one or more recombinant invasive gene(s) encode a HIV-1 glycoprotein 120 and a HIV-1 glycoprotein 41, in combination with a 1.B.12.8.2 family autotransporter-1.

15. A recombinant bacterial cell comprising recombinant genes encoding a fusion protein comprising an N-terminal signal peptide of an autotransporter antigen 43 (FLU) protein, an HIV-1 glycoprotein 120, a first linker peptide, an HIV-1 glycoprotein 41, and a second linker, an autochaperone (AC1) domain and a β-chain translocator domain of said autotransporter antigen, fused in consecutive order.

16. The recombinant bacterial cell according to claim 15, wherein the amino acid sequence of the HIV-1 glycoprotein 120, the first linker peptide, and the HIV-1 glycoprotein 41 has at least 80% sequence identity to SEQ ID NO.: 10.

17. The recombinant bacterial cell according to claim 15, wherein the amino acid sequence of the fusion protein has at least 80% sequence identity to SEQ ID No.: 284.

18. The recombinant bacterial cell according to claim 15, wherein said recombinant genes encoding the fusion protein are located on a plasmid.

19. (canceled)
